(19) United States
(12) Patent Application Publication
(10) Pub. No.: US 2026/0074025 A1
(43) Pub. Date: Mar. 12, 2026

(54) THREE-DIMENSIONAL MOLECULE GENERATION IN LATENT VOXELIZED SPACE

(71) Applicants: Genentech, Inc., South San Francisco, CA (US); Hoffmann-La Roche Inc., Little Falls, NJ (US)

(72) Inventors: Michael R. MASER, South San Francisco, CA (US); Ewa Madgalena NOWARA, South San Francisco, CA (US); Pedro Henrique OLIVEIRA PINHEIRO, Brooklyn, NY (US); Saeed SAREMI, San Francisco, CA (US)

(21) Appl. No.: 19/390,374

(22) Filed: Nov. 14, 2025

Related U.S. Application Data
(63) Continuation of application No. PCT/US2024/029662, filed on May 16, 2024.
(60) Provisional application No. 63/502,529, filed on May 16, 2023.
(60) Provisional application No. 63/586,263, filed on Sep. 28, 2023.
(60) Provisional application No. 63/623,062, filed on Jan. 19, 2024.

(51) Int. Cl. G16C 20/50; G16C 20/70

(52) U.S. Cl. CPC G16C 20/50; G16C 20/70

(57) ABSTRACT

A voxelized representation of an input molecule may be encoded to generate an embedding of the input molecule having a fewer quantity of features than the voxelized representation of the input molecule. A molecule design computation model may be applied to update the embedding of the input molecule. The molecule design computation model may be trained to approximate a data distribution of molecules exhibiting one or more desired properties by ingesting as input a corrupted embedding of a voxelized representation of a sample molecule exhibiting the one or more desired properties and recovering an embedding of the voxelized representation of the sample molecule. The molecule design computation model may update the embedding of the input molecule to increase a likelihood of a resultant updated embedding within the data distribution. A voxelized representation of an output molecule may be generated by at least decoding the resultant updated embedding.

### CROSS REFERENCE TO RELATED APPLICATIONS

This application claims priority to U.S. Provisional Application 63/502,529, entitled “THREE-DIMENSIONAL MOLECULE GENERATION BY DENOISING VOXEL GRIDS” and filed on May 16, 2023, U.S. Provisional Application No. 63/586,263, entitled “THREE-DIMENSIONAL MOLECULE GENERATION BY DENOISING VOXEL GRIDS” and filed on Sep. 28, 2023, and U.S. Provisional Application No. 63/623,062, entitled “THREE-DIMENSIONAL MOLECULE GENERATION BY DENOISING VOXEL GRIDS” and filed on Jan. 19, 2024, the disclosures of which are incorporated herein by reference in their entireties.

### TECHNICAL FIELD

The subject matter described herein relates generally to generative artificial intelligence and more specifically to machine learning enabled techniques for generating representations of three-dimensional molecules in discrete and latent voxelized space.

### INTRODUCTION

A molecule is a group of two more atoms held together by chemical bonds. Molecules form the smallest identifiable unit into which a pure substance can be divided while still retaining the composition and chemical properties of that substance. One example of a molecule is a small molecule, which is a low-weight compound having a molecular weight between approximately 100 Daltons and 1000 Daltons. Small molecule therapeutics, which modulate biochemical processes to diagnose, treat, and prevent a gamut of illnesses, have been a cornerstone in modern pharmacology due to a number of compelling advantages. For example, small molecule drugs are capable of penetrating cell membranes to reach intracellular targets. Moreover, small molecule drugs are adaptable to a wide variety of therapeutic applications. For instance, a small molecule drug may be formulated as pills and capsules, intravenous or subcutaneous injectables, inhalational medicines, or suppositories. The development of the small molecule drug may further extend to tailoring various pharmacokinetic properties including liberation, absorption, distribution, metabolism, potency, efficacy, phenotypic effects, and excretion.

By contrast, large molecules (also known as biopharmaceuticals, biologicals, or biologics) can range between approximately 3000 Daltons and 150,000 Daltons in molecular weight. Large molecule drugs are often derivatives of natural human proteins, which modulate many essential cellular functions such as enzymatic reactions, transport of molecules, regulation and execution of a number of biological pathways, cell growth, proliferation, nutrient uptake, morphology, motility, intercellular communication, and/or the like. It is common for a single large molecule to have more than 1,300 amino acid residues, which are linked by peptide bonds to form one or more polypeptide. Due to their size and complexity, large molecule drugs are recombinantly produced by engineered cells instead of being chemically synthesized like the majority of small molecule drugs. Moreover, large molecule therapeutics are usually delivered through injection or infusion due to the ineffectiveness of oral administration. The development of a large molecule drug may entail designing one or more sequences of amino acid residues capable of binding to a target (e.g., a protein, a nucleic acid, and/or the like) with sufficient specificity and absent undesirable traits such as immunogenicity, self-association, instability, and/or the like.

### SUMMARY

Systems, methods, and articles of manufacture, including computer program products, are provided for generating three-dimensional molecules in latent voxelized space. In one aspect, there is provided a system for machine learning enabled three-dimensional molecule generation. The system may include at least one processor and at least one memory. The at least one memory may include program code that provides operations when executed by the at least one processor. The operations may include: encoding a voxelized representation of an input molecule to generate an embedding of the input molecule having a fewer quantity of features than the voxelized representation of the input molecule; applying a molecule design computation model to update the embedding of the input molecule, where the molecule design computation model has been trained to approximate a data distribution of molecules exhibiting one or more desirable properties by ingesting as input a corrupted embedding of a voxelized representation of a sample molecule exhibiting the one or more desired properties and recovering an embedding of the voxelized representation of the sample molecule from the corrupted embedding, the molecule design computation model updating the embedding of the input molecule to increase a likelihood of a resultant updated embedding within the data distribution; and generating a voxelized representation of an output molecule by at least decoding the resultant updated embedding.

In another aspect, there is provided a method for machine learning enabled three-dimensional molecule generation. The method may include: encoding a voxelized representation of an input molecule to generate an embedding of the input molecule having a fewer quantity of features than the voxelized representation of the input molecule; applying a molecule design computation model to update the embedding of the input molecule, where the molecule design computation model has been trained to approximate a data distribution of molecules exhibiting one or more desirable properties by ingesting as input a corrupted embedding of a voxelized representation of a sample molecule exhibiting the one or more desired properties and recovering an embedding of the voxelized representation of the sample molecule from the corrupted embedding, the molecule design computation model updating the embedding of the input molecule to increase a likelihood of a resultant updated embedding within the data distribution; and generating a voxelized representation of an output molecule by at least decoding the resultant updated embedding.

In another aspect, there is provided a computer program product for machine learning enabled three-dimensional molecule generation. The computer program product may include a non-transitory computer readable medium storing instructions that cause operations when executed by at least one data processor. The operations may include: encoding a voxelized representation of an input molecule to generate an embedding of the input molecule having a fewer quantity of features than the voxelized representation of the input molecule; applying a molecule design computation model to update the embedding of the input molecule, where the molecule design computation model has been trained to approximate a data distribution of molecules exhibiting one or more desirable properties by ingesting as input a corrupted embedding of a voxelized representation of a sample molecule exhibiting the one or more desired properties and recovering an embedding of the voxelized representation of the sample molecule from the corrupted embedding, the molecule design computation model updating the embedding of the input molecule to increase a likelihood of a resultant updated embedding within the data distribution; and generating a voxelized representation of an output molecule by at least decoding the resultant updated embedding.

In some variations, one or more features disclosed herein including the following features can optionally be included in any feasible combination.

In some variations, the data distribution may be a noisy data distribution populated by noisy embeddings of a voxelized representations of the molecules exhibiting the one or more desirable properties. The voxelized representation of the output molecule may be further generated by denoising a noisy voxelized representation of the output molecule generated by the decoding of the resultant updated embedding.

In some variations, a vector quantized variational autoencoder (VQ-VAE) may be applied to encode the voxelized representation of the input molecule and to decode the resultant updated embedding.

In some variations, an embedding of a molecule may be a discrete latent embedding vector generated by quantizing a corresponding continuous latent embedding. The quantizing may include matching the corresponding continuous latent embedding to a vector in a codebook of embeddings by a nearest neighbor lookup.

In some variations, the voxelized representation of the input molecule may be encoded by at least compressing a plurality of atomic density values comprising the voxelized representation of the input molecule such that the embedding of the input molecule includes fewer features than the voxelized representation of the input molecule.

In some variations, a voxelized representation of a molecule may include a plurality of voxels organized into a three-dimensional voxel grid. Each atom in the molecule may be represented as a continuous density across one or more voxels in the three-dimensional voxel grid.

In some variations, the continuous density of each atom in the molecule may be centered at a center of each atom. A first voxel located distanced from any atoms in the molecule may be associated with a lower atomic density value than a second voxel located proximate to the center of an atom in the molecule.

In some variations, each voxel in the three-dimensional voxel grid may be associated with a value indicative of an atomic density at a corresponding location.

In some variations, a voxelized representation of a molecule may include one or more channels. Each channel may correspond to a type of atom present in the molecule.

In some variations, a voxelized representation of a molecule may jointly represent a type and a position of one or more atoms present in the molecule.

In some variations, the embedding of the input molecule may be updated based at least on a function parameterized by a plurality parameters of the molecule design computation model. The function may output a value indicative of a likelihood of the resultant updated embedding within the data distribution.

In some variations, the function may be a score function. The value output by the function may be a score indicating a local change in a density of the noisy data distribution at a location of each updated noisy embedding generated by the updating the noisy embedding of the input molecule.

In some variations, the molecule design computation model may update the embedding of the input molecule by at least applying the molecule design computation model to update the embedding of the input molecule thereby generating a first updated embedding, applying the molecule design computation model to update the embedding of the input molecule thereby generating a second updated embedding, applying a function parameterized by a plurality of parameters of the molecule design computation model to determine (i) a first value indicative of a first local change in a density of the data distribution at a first location occupied by the first updated embedding and (ii) a second value indicative of a second local change in the density of the data distribution at a second location occupied by the second updated embedding, and applying the molecule design computation model to further update, based at least on the first value and the second value, the first updated embedding instead of the second updated embedding

In some variations, the molecule design computation model may be applied to further update the first updated embedding until one or more criteria are met. The one or more criteria may include at least one of (i) a threshold quantity of iterations of updates to the embedding of the input molecule have been performed, (ii) the first value of the first updated embedding satisfying one or more thresholds, and (iii) a threshold quantity of output molecules have been generated.

In some variations, the molecule design computation model may be applied to further modify the first updated embedding instead of the second updated embedding based at least on the first value and the second value indicating that the first updated embedding has a higher likelihood within the data distribution than the second updated embedding.

In some variations, the molecule design computation model may be applied to further modify the first updated embedding instead of the second updated embedding based at least on the first value and the second value indicating that the first updated embedding is sampled from a higher density region of the data distribution than the second updated embedding.

In some variations, the voxelized representation of the output molecule may be translated into a one-dimensional representation of the output molecule and/or a two-dimensional representation of the output molecule.

In some variations, the voxelized representation of the output molecule may be translated by at least determining a position of one or more atoms in the output molecule by at least detecting one or more peaks in a plurality of atomic density values comprising the voxelized representation of the output molecule, and determining, based at least the positions of the one or more atoms, one or more interconnecting bonds.

Systems, methods, and articles of manufacture, including computer program products, are provided for generating three-dimensional molecules in latent voxelized space. In one aspect, there is provided a system for machine learning enabled three-dimensional molecule generation. The system may include at least one processor and at least one memory. The at least one memory may include program code that provides operations when executed by the at least one processor. The operations may include: generating a training dataset to include a plurality of training samples, each training sample in the training dataset including a corrupted embedding generated by at least adding noise to a noisy voxelized representation of a sample molecule exhibiting one more desirable properties; training, based at least on the training dataset, a molecule design computation model to approximate a data distribution of molecules exhibiting the one or more desirable properties, the training includes applying the molecule design computation model to recover, from the corrupted embedding of the noisy voxelized representation of the sample molecule, an uncorrupted embedding of the noisy voxelized representation of the sample molecule; and optionally applying the molecule design computation model to generate an output molecule by at least denoising an embedding of a voxelized representation of an input molecule and decoding an updated embedding resulting therefrom to generate a voxelized representation of the output molecule.

In another aspect, there is provided a method for machine learning enabled three-dimensional molecule generation. The method may include: generating a training dataset to include a plurality of training samples, each training sample in the training dataset including a corrupted embedding generated by at least adding noise to a noisy voxelized representation of a sample molecule exhibiting one more desirable properties; training, based at least on the training dataset, a molecule design computation model to approximate a data distribution of molecules exhibiting the one or more desirable properties, the training includes applying the molecule design computation model to recover, from the corrupted embedding of the noisy voxelized representation of the sample molecule, an uncorrupted embedding of the noisy voxelized representation of the sample molecule; and optionally applying the molecule design computation model to generate an output molecule by at least denoising an embedding of a voxelized representation of an input molecule and decoding an updated embedding resulting therefrom to generate a voxelized representation of the output molecule.

In another aspect, there is provided a computer program product for machine learning enabled three-dimensional molecule generation. The computer program product may include a non-transitory computer readable medium storing instructions that cause operations when executed by at least one data processor. The operations may include: generating a training dataset to include a plurality of training samples, each training sample in the training dataset including a corrupted embedding generated by at least adding noise to a noisy voxelized representation of a sample molecule exhibiting one more desirable properties; training, based at least on the training dataset, a molecule design computation model to approximate a data distribution of molecules exhibiting the one or more desirable properties, the training includes applying the molecule design computation model to recover, from the corrupted embedding of the noisy voxelized representation of the sample molecule, an uncorrupted embedding of the noisy voxelized representation of the sample molecule; and optionally applying the molecule design computation model to generate an output molecule by at least denoising an embedding of a voxelized representation of an input molecule and decoding an updated embedding resulting therefrom to generate a voxelized representation of the output molecule.

In some variations, one or more features disclosed herein including the following features can optionally be included in any feasible combination.

In some variations, the noisy voxelized representation of the sample molecule may include a plurality voxels organized into a three-dimensional voxel grid. Each atom in the sample molecule may be represented as a continuous density across one or more voxels in the three-dimensional voxel grid.

In some variations, the continuous density of each atom in the sample molecule may be centered at a center of each atom. A first voxel located distanced from any atoms in the sample molecule is associated with a lower atomic density value than a second voxel located proximate to the center of an atom in the sample molecule.

In some variations, each voxel in the three-dimensional voxel grid may be associated with a value indicative of an atomic density at a corresponding location.

In some variations, the noisy voxelized representation of the sample molecule may include one or more channels. Each channel may correspond to a type of atom present in the sample molecule.

In some variations, the noisy voxelized representation of the sample molecule may jointly represent a type and a position of one or more atoms present in the sample molecule.

In some variations, the training of the molecule design computation model may include adjusting a plurality of parameters of the molecule design computation model to reduce a difference between a recovered embedding generated by the molecule design computation model and the uncorrupted embedding of the noisy voxelized representation of the sample molecule.

In some variations, the plurality of parameters of the molecule design computation model may parameterize a function. The plurality of parameters may be adjusted such that the function outputs a value indicative of a local change in a density of the data distribution of molecules exhibiting the one or more desirable properties.

In some variations, the molecule design computation model may denoise the embedding of the voxelized representation of the input molecule by at least updating at least one value present in the embedding that is representative of multiple atomic density values present in the voxelized representation of the input molecule.

In some variations, updating an atomic density value of the one or more voxels in the at least one channel of the voxelized representation of the input molecule may correspond to updating at least one of a type and/or a position of one or more atoms present in the input molecule.

In some variations, the molecule design computation model may denoise the embedding of the voxelized representation of the input molecule over multiple iterations of gradient based Markov Chain Monte Carlo (MCMC) sampling until one or more criteria are satisfied.

In some variations, the one or more criteria may include at least one of (i) a threshold quantity of iterations of gradient-based Markov Chain Monte Carlo (MCMC) sampling have been performed, (ii) the resultant updated embedding is sampled from a region having a threshold density, and (iii) a threshold quantity of output molecules have been generated.

In some variations, the molecule design computation model may generate the voxelized representation of the output molecule by at least applying a first update to the embedding of the voxelized representation of the input molecule to generate a first updated embedding, applying a second update to the embedding of the voxelized representation of the input molecule to generate a second updated embedding, and upon determining that the first updated embedding is sampled from a higher density region of the data distribution than the second updated embedding, further updating first updated embedding instead of the second updated embedding.

In some variations, the voxelized representation of the output molecule may be translated into a one-dimensional representation and/or a two-dimensional representation of the output molecule.

In some variations, the training of the molecule design computation model may include applying the molecule design computation model having a first adjustment to generate a first recovered embedding of the noisy voxelized representation of the sample molecule, determining a first mean squared error (MSE) quantifying a first difference between the first recovered embedding and the uncorrupted embedding of noisy voxelized representation of the sample molecule, applying the molecule design computation model having a second adjustment to generate a second recovered embedding of the noisy voxelized representation of the sample molecule, determining a second mean squared error (MSE) quantifying a second difference between the second recovered embedding and the uncorrupted embedding of noisy voxelized representation of the sample molecule, and upon determining that the first mean squared error (MSE) is less than the second mean squared error (MSE), further adjusting the molecule design computation model having the first adjustment instead of the second adjustment.

In some variations, the molecule design computation model may be further adjusted until one or more criteria are met. The one or more criteria may include at least one of (i) a threshold quantity of iterations of adjustments to the molecule design computation model have been performed, and (ii) a recovered embedding exhibiting a threshold mean squared error (MSE) value has been generated.

In some variations, an autoencoder comprising an encoder and a decoder may be trained. The training of the autoencoder may include training the encoder to encode the noisy voxelized representation of the sample molecule such that the decoder is able to recover the voxelized representation of the sample molecule from a resulting embedding of the noisy voxelized representation of the sample molecule.

In some variations, the autoencoder may be a vector quantized variational autoencoder (VQ-VAE) in which the encoder generates a continuous latent embedding of the sample molecule that is then quantized, by being matched to a vector in a codebook of embeddings by a nearest neighbor lookup, to a discrete latent embedding for decoding by the decoder.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying selectings and the description below. Other features and advantages of the subject matter described herein will be apparent from the description and selectings, and from the claims. While certain features of the currently disclosed subject matter are described for illustrative purposes in relation to the computational design of molecules including drug molecules, it should be readily understood that such features are not intended to be limiting. The claims that follow this disclosure are intended to define the scope of the protected subject matter.

### DESCRIPTION OF SELECTINGS

The accompanying selectings, which are incorporated in and constitute a part of this specification, show certain aspects of the subject matter disclosed herein and, together with the description, help explain some of the principles associated with the disclosed implementations. In the selectings,

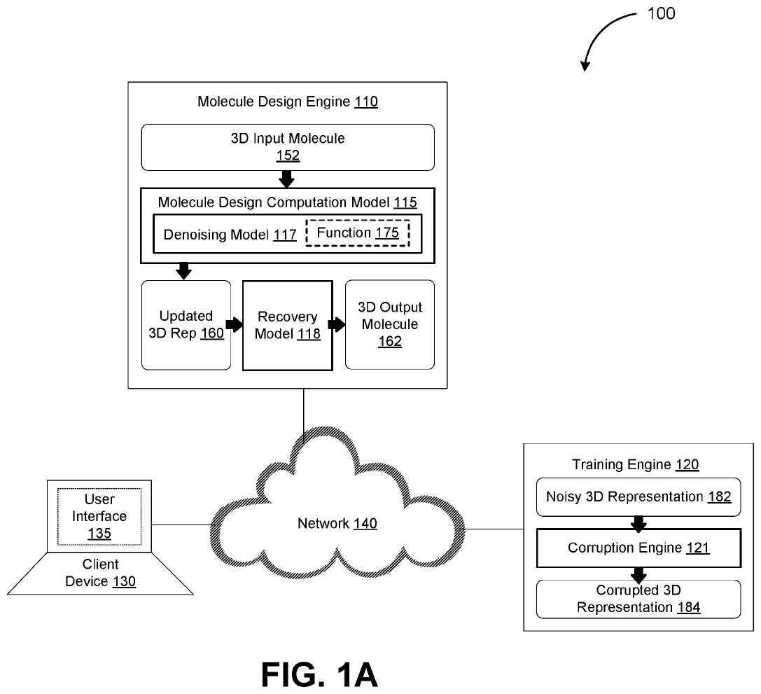

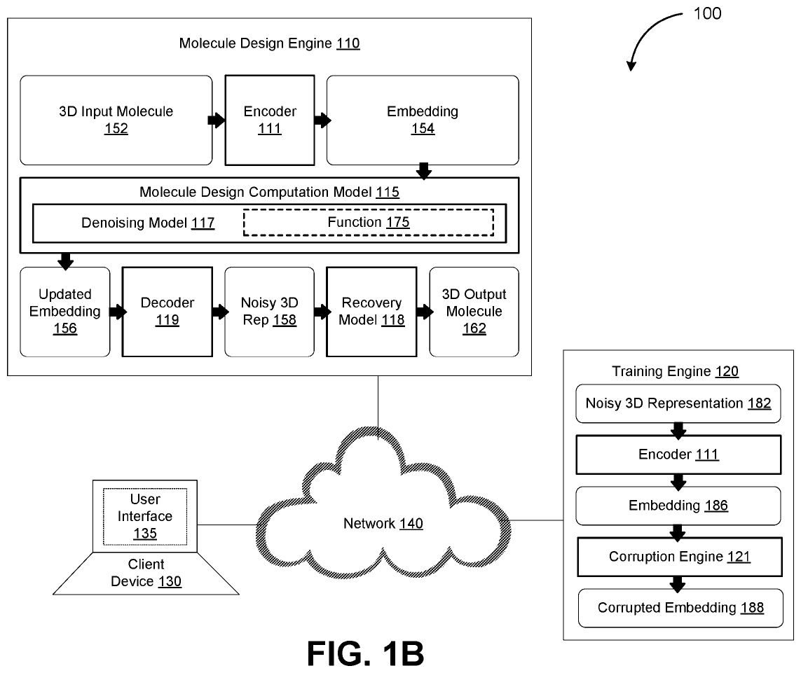

FIG. 1A depicts a system diagram illustrating an example of a molecule design system, in accordance with some example embodiments;

FIG. 1B depicts a system diagram illustrating another example of a molecule design system, in accordance with some example embodiments;

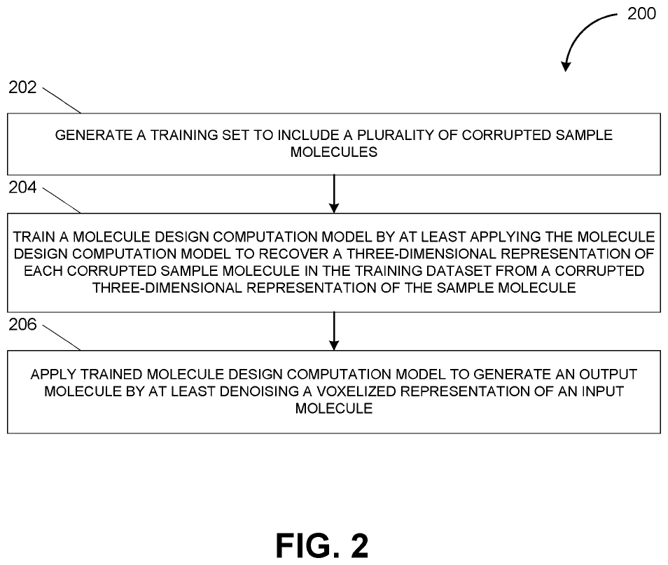

FIG. 2 depicts a flowchart illustrating an example of a process for machine learning enabled generation of three-dimensional molecules in voxelized space, in accordance with some example embodiments;

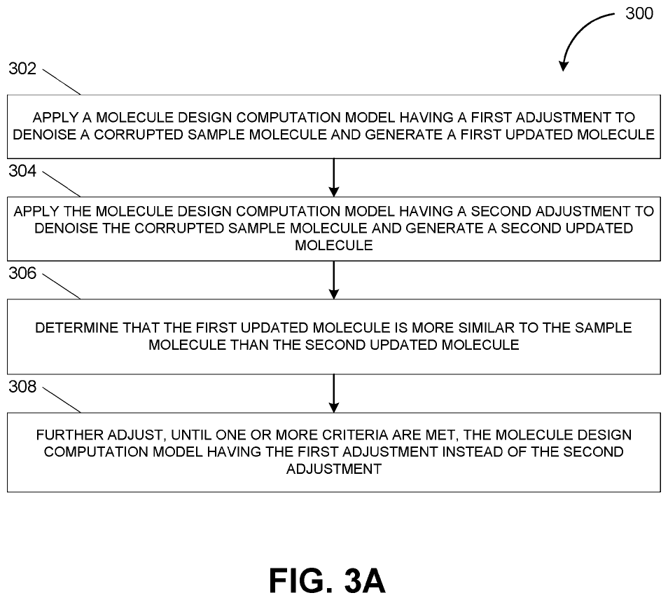

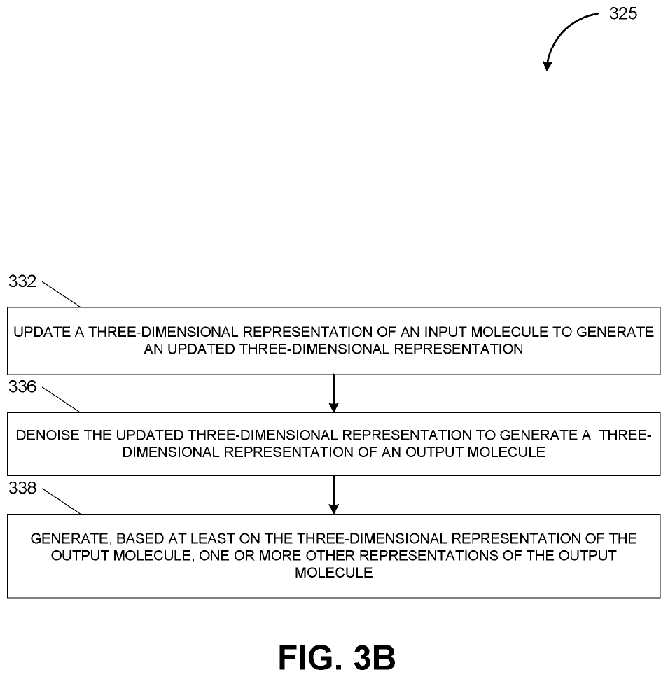

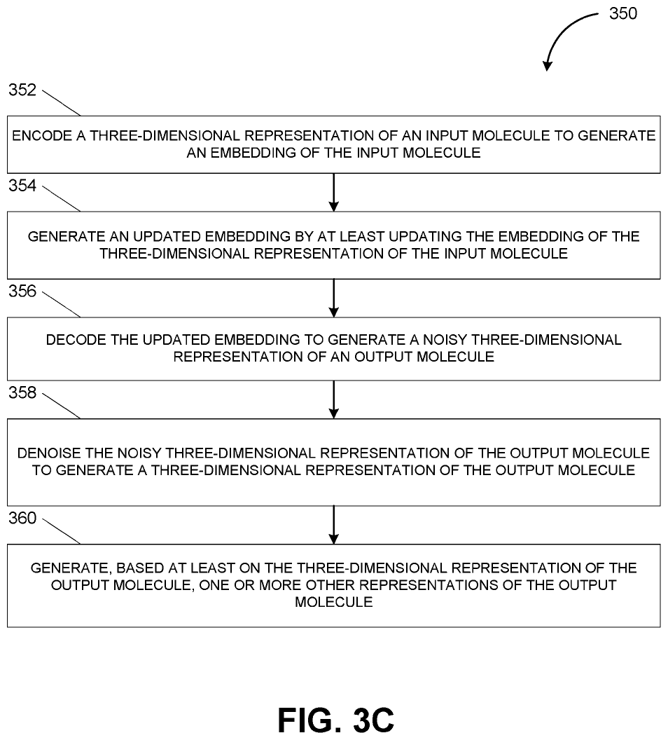

FIG. 3A depicts a flowchart illustrating an example of a process for training a molecule design computation model to generate three-dimensional molecules in voxelized space, in accordance with some example embodiments;

FIG. 3B depicts a flowchart illustrating an example of a process for applying a molecule design computation model to generate three-dimensional molecules in voxelized space, in accordance with some example embodiments;

FIG. 3C depicts a flowchart illustrating an example of a process for applying a molecule design computation model to generate three-dimensional molecules in voxelized space, in accordance with some example embodiments;

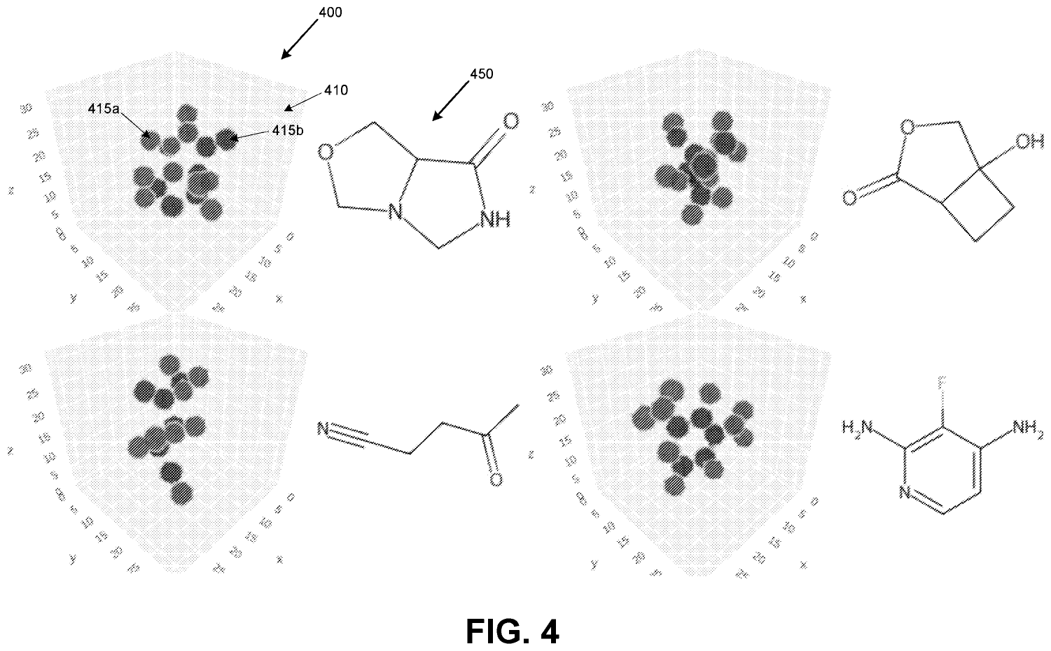

FIG. 4 depicts an example of a voxelized representation of a molecule, in accordance with some example embodiments;

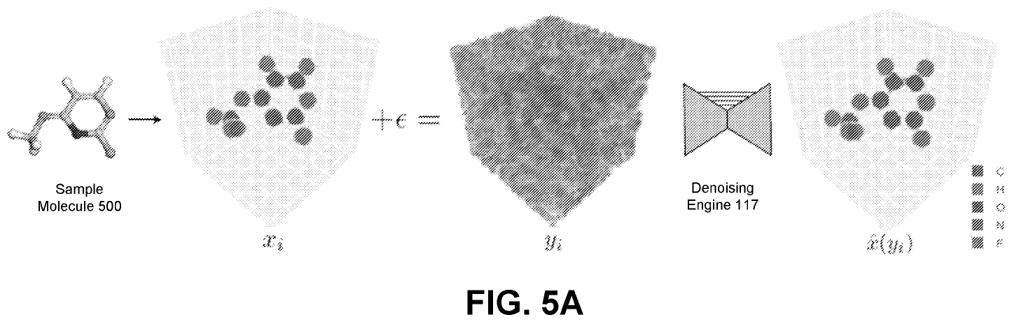

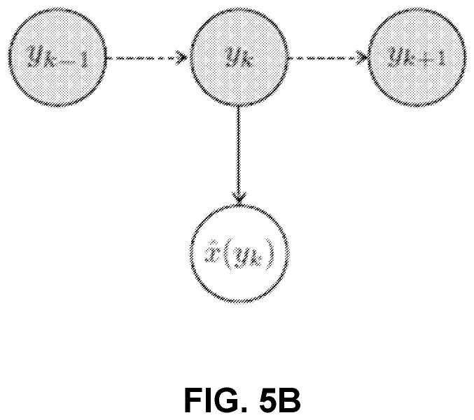

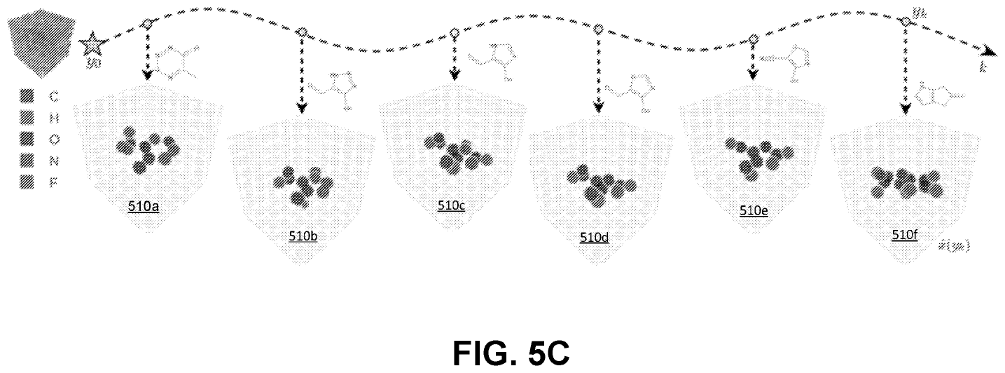

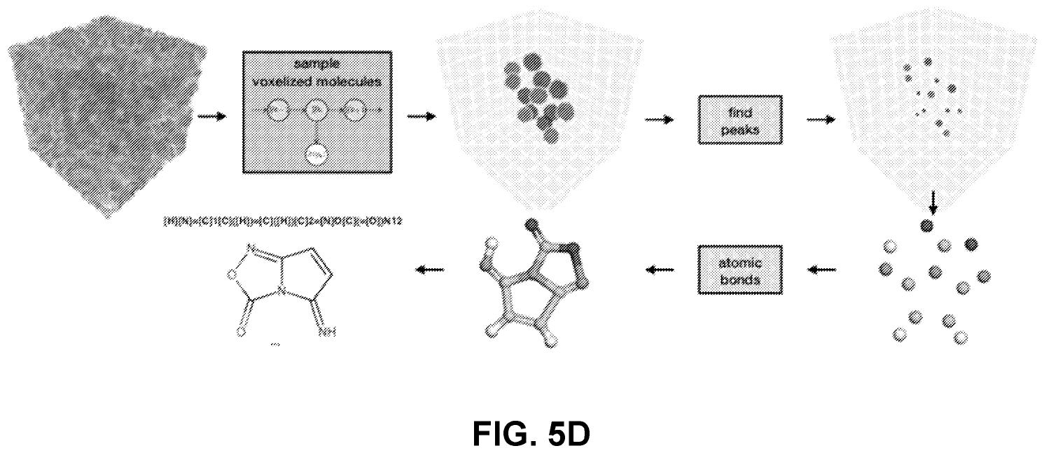

FIG. 5A depicts a schematic diagram illustrating an example of a process for a training a denoising engine to denoise noisy voxelized representations of molecules, in accordance with some example embodiments;

FIG. 5B depicts a schematic diagram illustrating an example of a walk-jump sampling scheme, in accordance with some example embodiments;

FIG. 5C depicts a schematic diagram illustrating an example of a process for a molecule design computation model to generate three-dimensional molecules by denoising voxelized molecule representations, in accordance with some example embodiments;

FIG. 5D depicts a schematic diagram illustrating an example of a process for generating other molecular representations from the voxelized representation of a molecule, in accordance with some example embodiments;

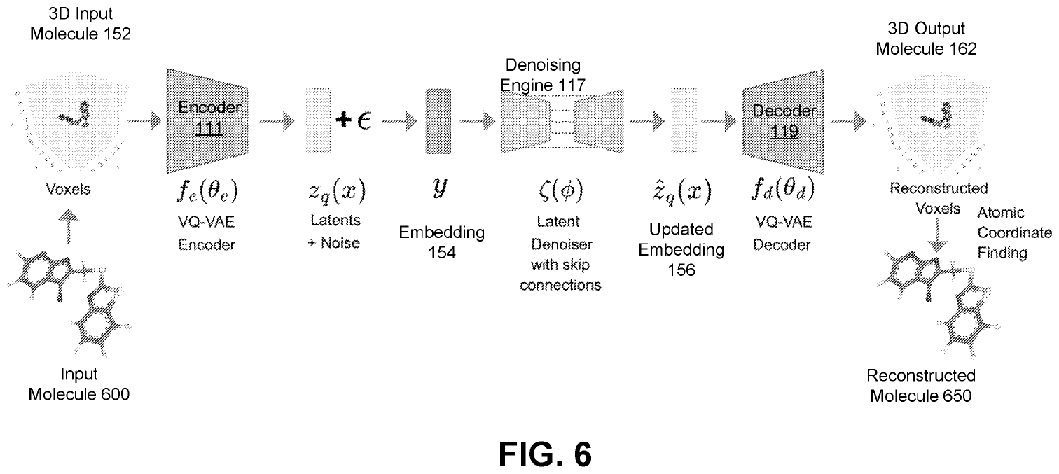

FIG. 6 depicts a schematic diagram illustrating an example of a process for a molecule design computation model to generate voxelized representations of molecules by operating in a noisy latent voxelized space, in accordance with some example embodiments;

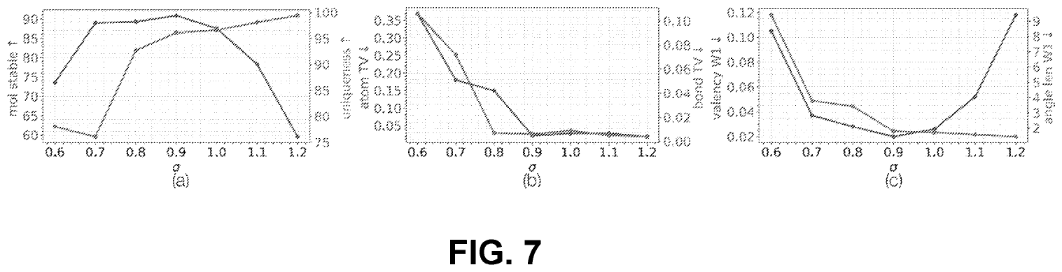

FIG. 7 depicts graphs illustrating the effect of noise level on the generative performance of a molecule design computation model, in accordance with some example embodiments;

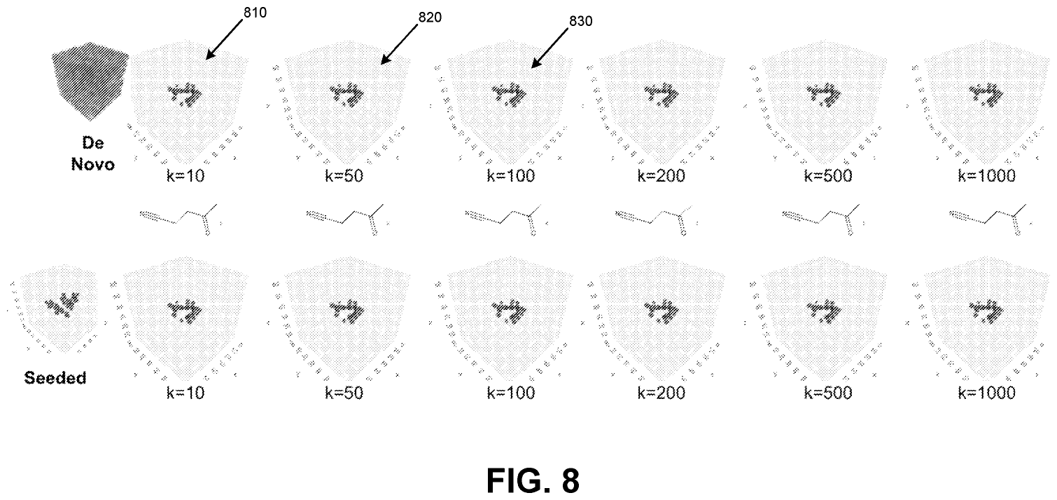

FIG. 8 depicts a schematic diagram illustrating the effects of the number of sampling iterations in Markov Chain Monte Carlo (MCMC) sampling on the generative performance of a molecule design computation model, in accordance with some example embodiments;

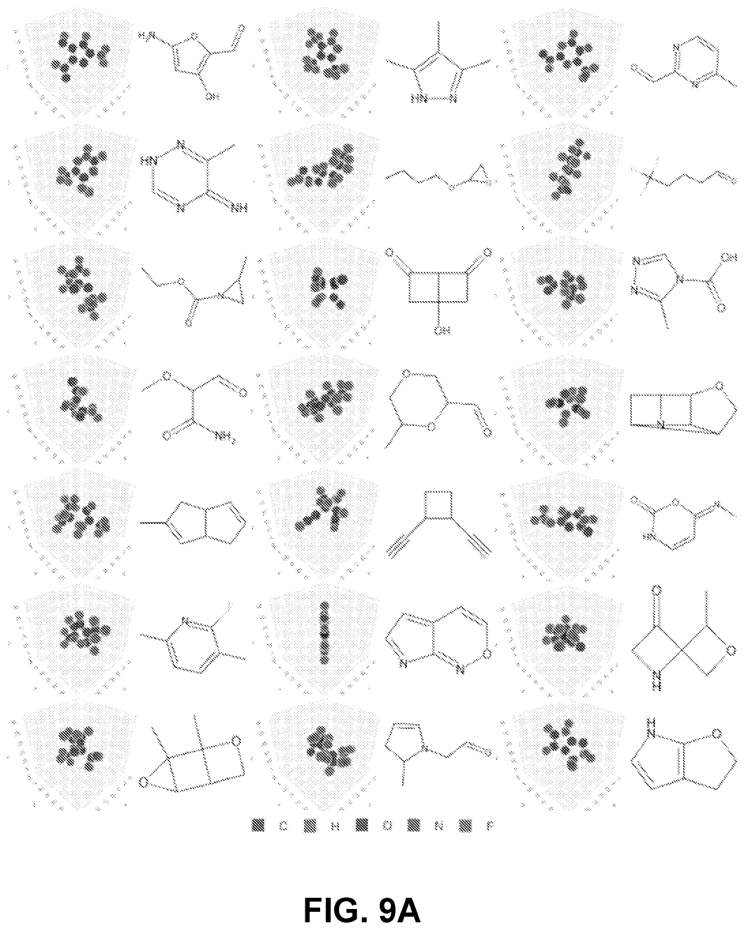

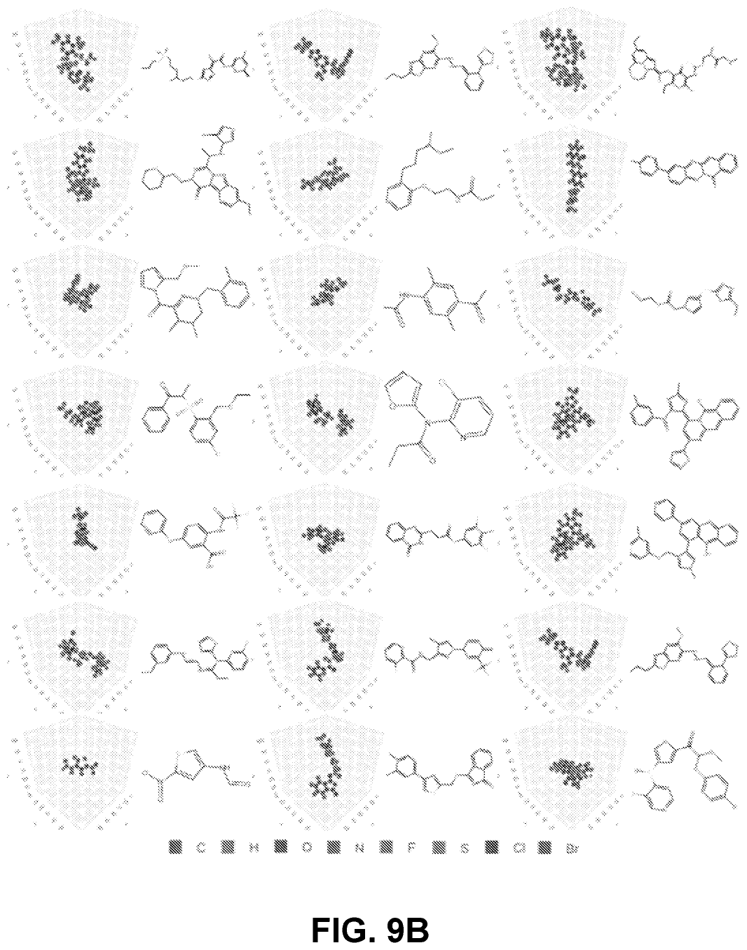

FIG. 9A depicts examples of voxelized representations of molecules generated by a molecule design computation model trained on the QM9 molecule dataset, in accordance with some example embodiments;

FIG. 9B depicts examples of voxelized representations of molecules generated by a molecule design computation model trained on the Geometric Ensemble of Molecules (GEOM) Drugs dataset, in accordance with some example embodiments;

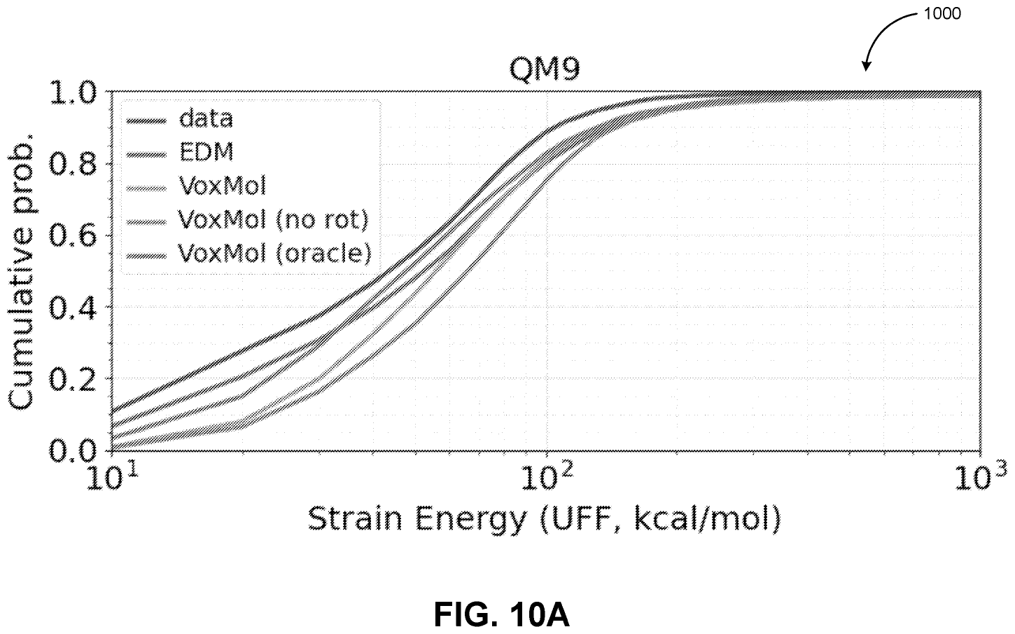

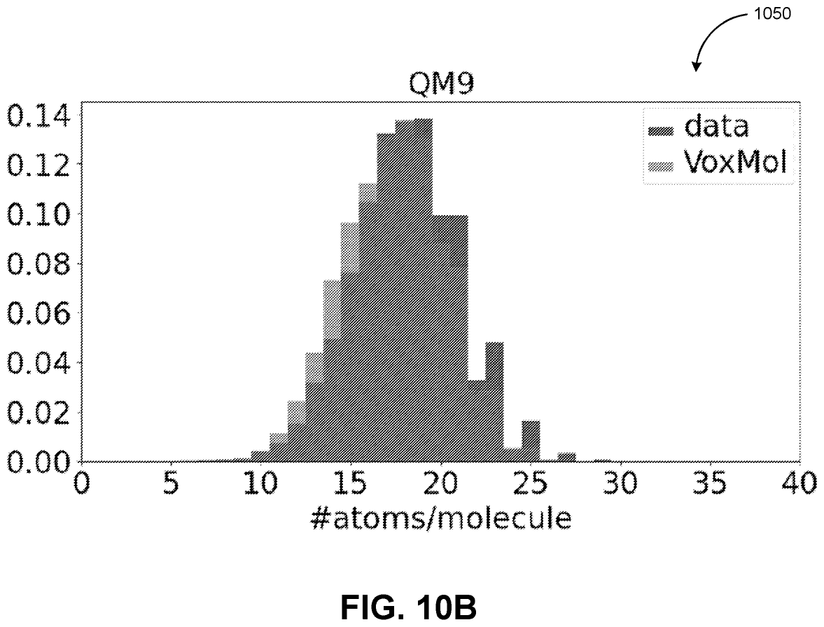

FIG. 10A depicts a graph illustrating the cumulative distribution function (CDF) of the strain energies of the molecules in the QM9 molecule dataset, the molecules generated by conventional generative models and the molecules generated by a molecule design computation model trained on the QM9 molecule dataset, in accordance with some example embodiments;

FIG. 10B depicts a graph illustrating the empirical distribution of the number of atoms per molecule in the QM9 molecule dataset compared to the empirical distribution of the number of atoms in the molecules generated by a molecule design computation model trained on the QM9 molecule dataset, in accordance with some example embodiments;

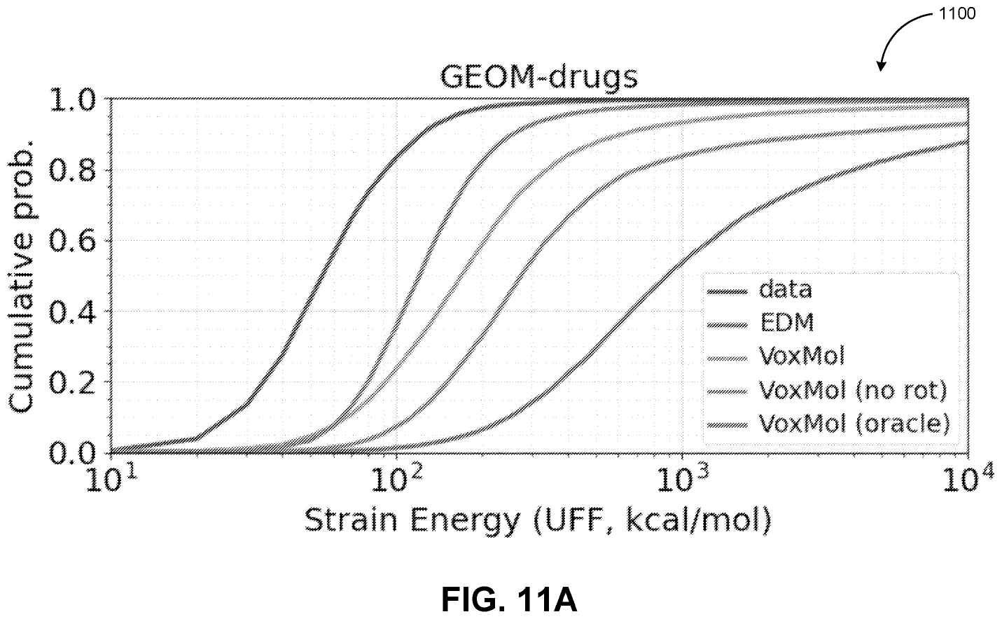

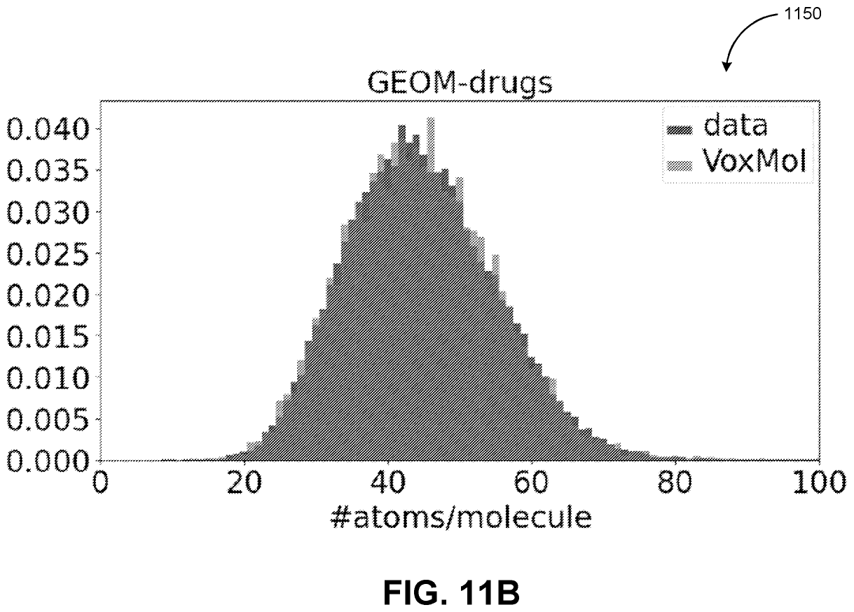

FIG. 11A depicts a graph illustrating the cumulative distribution function (CDF) of the strain energies of the molecules in the Geometric Ensemble of Molecules (GEOM) Drugs dataset, the molecules generated by conventional generative models and the molecules generated by a molecule design computation model trained on the GEOM Drugs dataset, in accordance with some example embodiments;

FIG. 11B depicts a graph illustrating the empirical distribution of the number of atoms per molecule in the Geometric Ensemble of Molecules (GEOM) Drugs dataset compared to the empirical distribution of the number of atoms in the molecules generated by a molecule design computation model trained on the GEOM Drugs dataset, in accordance with some example embodiments; and

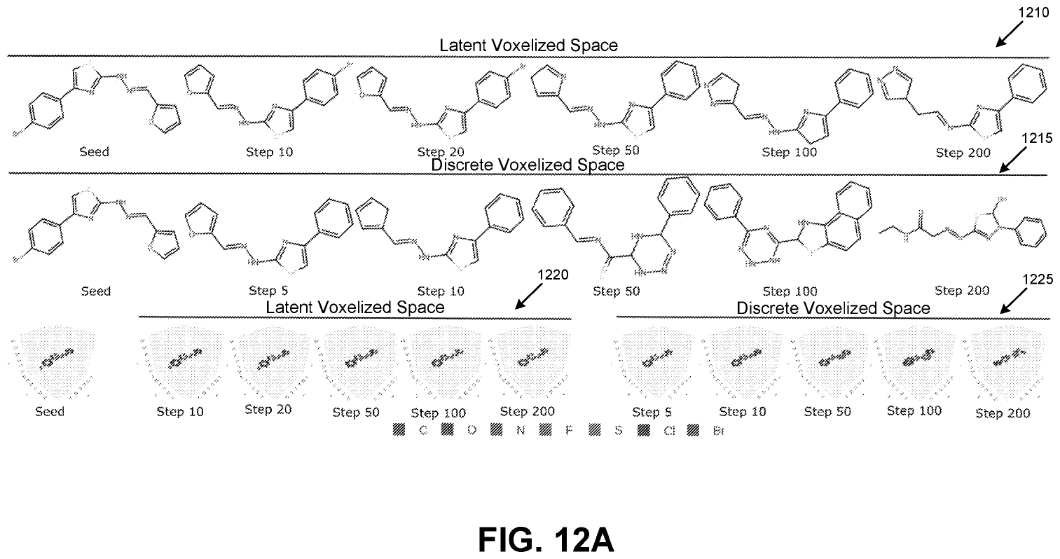

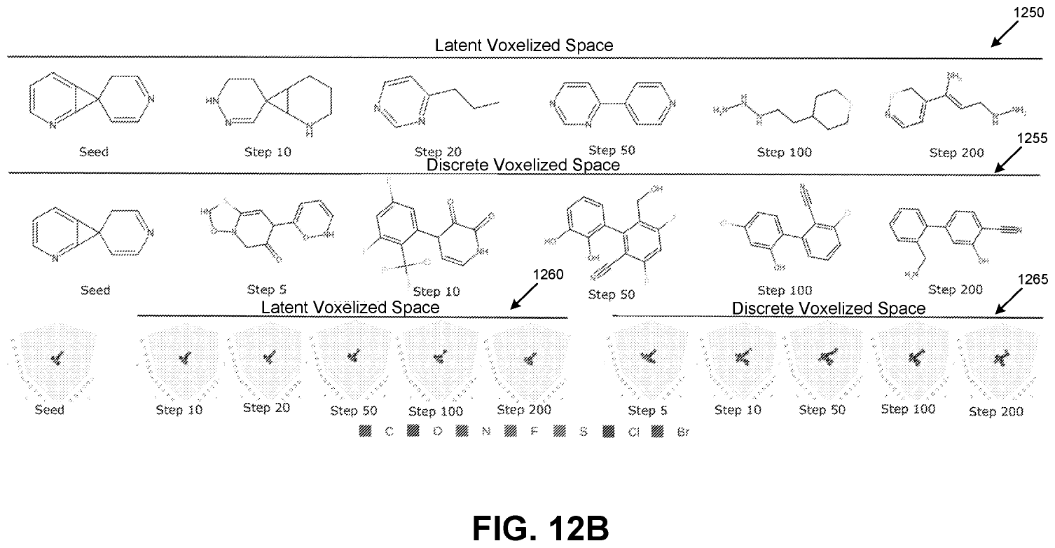

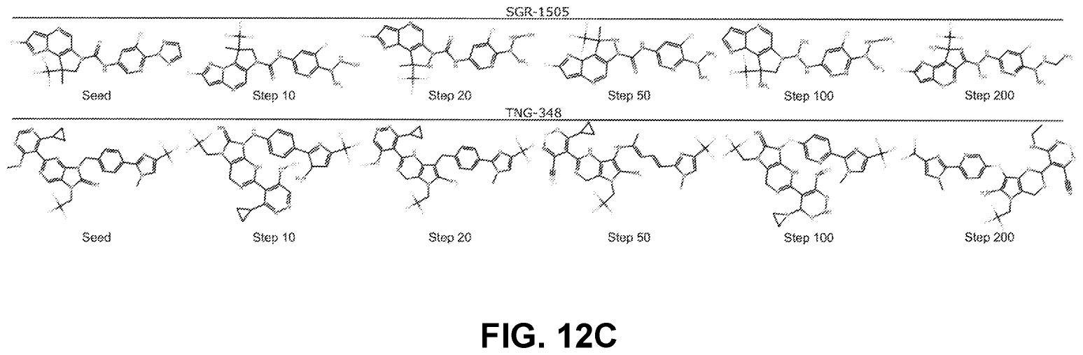

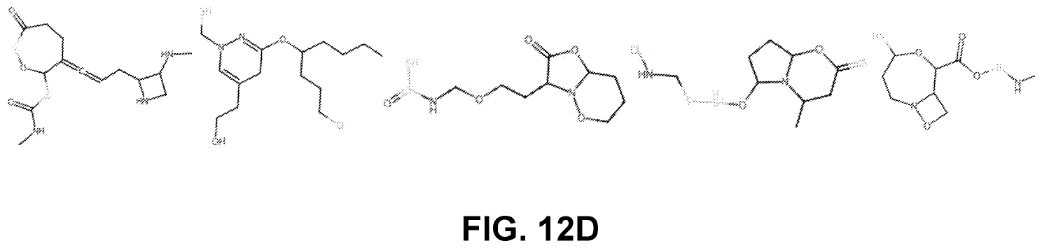

FIG. 12A depicts a schematic diagram illustrating a comparison of seeded generation performance on Geometric Ensemble of Molecules (GEOM) Drugs in discrete voxelized space and latent voxelized space, in accordance with some example embodiments;

FIG. 12B depicts a schematic diagram illustrating a comparison of seeded generation performance on PubChem drugs in discrete voxelized space and latent voxelized space, in accordance with some example embodiments;

FIG. 12C depicts the molecular graphs of additional examples of molecules generated by performing seeded generation in latent voxelized space, on real drugs.

FIG. 12D depicts the molecular graphs of additional examples of molecules generated by performing de novo generation in latent voxelized space, on real drugs.

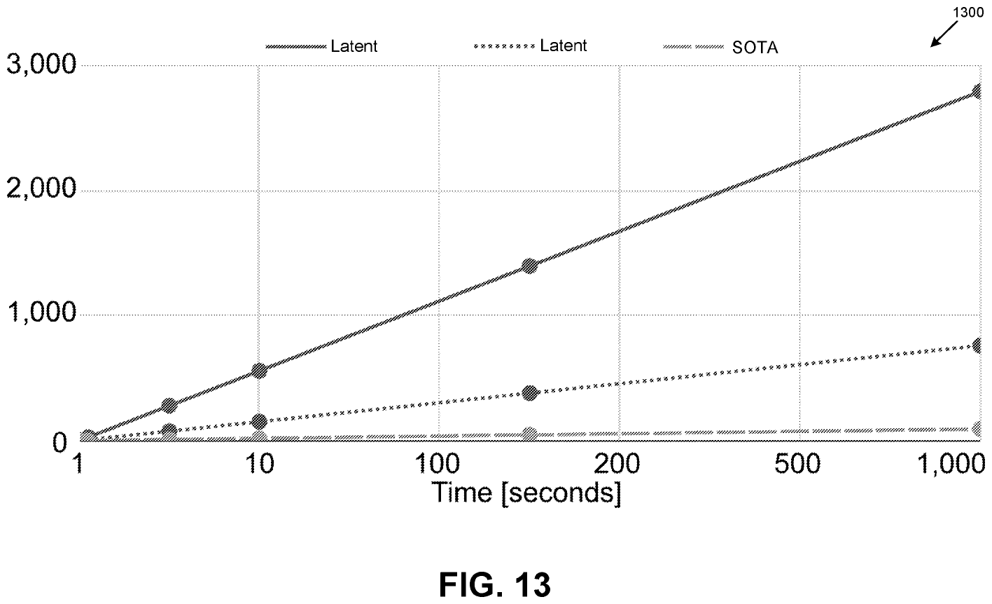

FIG. 13 depicts a graph illustrating a comparison of seeded generation performance on Geometric Ensemble of Molecules (GEOM) Drugs at in discrete voxelized space and latent voxelized space, in accordance with some example embodiments;

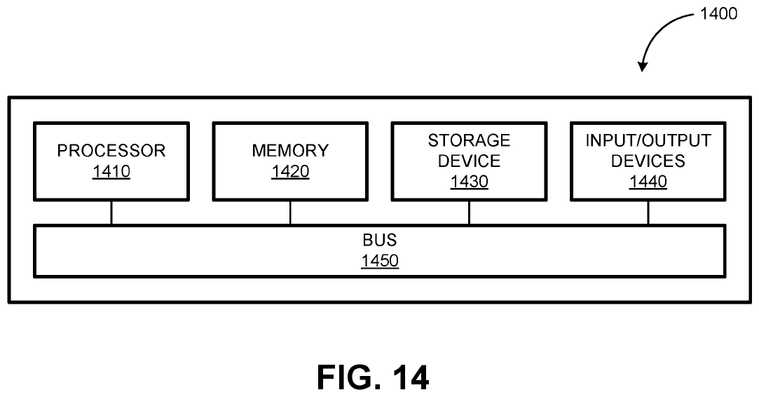

FIG. 14 depicts a block diagram illustrating an example of a computing system, in accordance with some example embodiments.

When practical, similar reference numbers denote similar structures, features, or elements.

### DETAILED DESCRIPTION

Generating new molecules with desired properties is a critical task in chemistry with applications across many scientific domains. In the context of drug discovery, conventional computational techniques for generating molecules with drug-like properties require conducting a search of the molecular space (or chemical space) occupied by every possible chemical compound (e.g., every possible combination of atoms of two or more chemical elements). For example, some search-based approaches may include scoring and ranking different molecules in the molecular space based on one or more drug-like properties, such as affinity, specificity, biological activity, and developability. However, the aforementioned molecular space, which is estimated to contain 10⁶⁰ possible chemical compounds, is prohibitively large and scales exponentially with molecule size (e.g., the number of constituent atoms). Even a very small portion of the molecular space can contain on the order of billions and trillions of molecules. With state-of-the-art computational resources, conventional search-based approaches are capable of exploring only a small fraction of the molecular space, such as small regions of the molecular space selected based on prior domain knowledge. This limitation in search scope means that conventional search-based approaches are likely to overlook molecules with more optimal properties. Moreover, conventional search-based approaches do not explore the molecular space in a principled manner, which prevents the generative process from being conditioned upon specific properties.

In addition, whether a molecule exhibits certain desired properties may be contingent on the conformation (or three-dimensional structure) of the molecule. For example, the binding affinity between a drug molecule and a target molecule (e.g., a protein, a nucleic acid, and/or the like) may depend on the ability of the drug molecule to adopt a conformation (or three-dimensional structure) that is complementary to that of the target molecule. Furthermore, molecules are flexible, meaning that a single molecule may assume one of numerous possible conformations (or three-dimensional structures). In some cases, a population of the same molecule can exist as an ensemble of many different conformations in equilibrium with one another but not every possible conformation is associated with desired properties. In the context of binding affinity, for instance, the biologically active conformation of a molecule may be one or more of the conformations exhibited by the molecule in solution or a new conformation that is induced by interactions with the target molecule. However, a one-dimensional representation (e.g., a simplified molecular-input line-entry system (SMILES) string) or a two-dimensional representation (e.g., a molecular graph) of a molecule do not adequately capture the conformation (or three-dimensional structure) of the molecule. Thus, in cases where the molecule design computation model operates on a one-dimensional representation or a two-dimensional representation of the input molecule, the resulting output molecule may fail to exhibit the conformation (or three-dimensional structure) associated with the one or more desired properties.

Various example embodiments of the present disclosure may improve upon the current state-of-the art computational resources by providing a molecule design computation model that may generate an output molecule through exploring the molecular space (or chemical space) in a principled manner, instead of an indiscriminate search of a limited portion of the molecular space. For example, in some cases, the molecule design computation model may be trained to approximate the data distribution of molecules exhibiting one or more desired properties (e.g., drug-like properties such as affinity, specificity, biological activity, developability, and/or the like). The training of the molecule design computation model may include determining the parameters of a function (e.g., a score function) such that the output of the function is a value indicative of the changes in density across the data distribution. In some cases, the molecule design computation model may sample the data distribution in order generate the output molecule to also exhibit the one or more desired properties. For instance, in some cases, the molecule design computation model may sample the data distribution by denoising the input molecule, such as the voxelized representation of the input molecule, over multiple sampling iterations. During each sampling iteration, the molecule design computation model may update the input molecule to remove a portion of the noise present in the input molecule. Doing so may generate an updated molecule (e.g., a voxelized representation of the updated molecule) that constitutes a sample selected from the data distribution. As described in more details below, the sampling may be guided by the function such that each successive sample (or updated molecule) is selected from an incrementally higher density region of the data distribution, which are more likely to be occupied by molecules exhibiting the one or more properties.

In some example embodiments, the likelihood that the output molecule exhibits the one or more desired properties may be increased (or maximized) by the molecule design computation model operating on a three-dimensional representation of the input molecule. For example, in some cases, the molecule design computation model may generate the output molecule by at least denoising, for example, over multiple sampling iterations, the three-dimensional representation of the input molecule. In some cases, the molecule design computation model may generate the output molecule by denoising a voxelized representation of the input molecule instead of a conventional three-dimensional representation of the input molecule. The conventional three-dimensional representation of the input molecule, such as a point-cloud representation of the input molecule, may specify the conformation (or three-dimensional structure) of the input molecule by at least specifying the coordinates (e.g., in Euclidean space) of the constituent atoms. However, the conventional three-dimensional representation of the input molecule may impose a number of limitations on the generative process. For instance, in order for the molecule design computation model to operate on the conventional three-dimensional representation of the input molecule, the number of atoms in the output molecule being generated therefrom must be known a priori. Denoising the conventional three-dimensional representation of the input molecule may also require certain work arounds in order for the molecule design computation model to be able to approximate the distribution of atom types in the output molecule, which forms a discrete distribution, whereas the positions of the atoms (e.g., atomic coordinates in Euclidean space) in the output molecule form a continuous distribution. Furthermore, the conventional three-dimensional representation of the input molecule may fail to adequately capture the long-range dependencies that exist across multiple atoms, especially as the quantity of constituent atoms increases.

In some example embodiments, the voxelized representation of a molecule (e.g. the input molecule) may obviate the aforementioned limitations by representing the input molecule as continuous distribution of atomic densities across voxel grids, centered around the atomic coordinates of each individual atom present in the molecule. For example, in a graph network representation of a molecule, the dependency between two adjacent atoms may be represented by an interconnecting edge. However, these edges may fail to adequate capture longer range dependencies, such as those between non-adjacent atoms. Contrastingly, the voxelized representation of the molecule may better capture long-range dependencies between distant atoms, even in instances where the input molecule contains a large quantity of atoms. Moreover, the molecule design computation model may operate on the voxelized representation of the input molecule to generate the output molecule without any a priori knowledge of the number of atoms present in the output molecule. This is because the molecule design computation model may be free to add or remove different types of atoms by updating the distribution of atomic densities across the voxel grids. The voxelized representation of the input molecule also jointly represents the types and positions of atoms in the input molecule, thereby obviating workarounds to reconcile the two different types of data distributions (e.g., discrete distribution for atom types and continuous distribution for atomic position).

In some example embodiments, the voxelized molecule representation of a molecule, such as the input molecule, may represent each atom in the molecule (e.g. input molecule) as a continuous (e.g., Gaussian-like) density across one or more voxels in the voxel grid. In this context, a voxel grid is a three-dimensional grid of voxels organized into contiguous layers of rows and columns. Various examples of the voxel grid described herein may contain multiple voxels, each of which being a volume element (e.g., a three-dimensional cube) at the intersection of a row and a column. Each volume element may have a predetermined size, which may or may not be the same for all of the voxels in the voxel grid. In cases where the input molecule is a drug molecule, the voxelized representation of the input molecule may include a voxel grid containing n×n×n voxels (e.g., 32×32×32 voxels, 64×64×64 voxels, and/or the like). In some cases, each voxel in the voxel grid may be associated with a value indicative of the atomic density at the corresponding location. For example, a first voxel associated with a higher atomic density may be more likely to be a portion of atom than a second voxel associated with a lower atomic density. It should be appreciated that the volume of an individual atom may span one or multiple voxels. In some cases, the atomic densities may also be centered around the atoms present in the input molecule, meaning that the atomic densities of an individual atom spanning multiple voxels may be centered on the voxel that comprises the center of that atom. A voxel having an atomic density of 0 may be far away from any atoms in the input molecule whereas a voxel having an atomic density of 1 may be at the center of an atom in the input molecule. Moreover, in some cases, the voxelized representation of a molecule (e.g. the input molecule) may include multiple channels, each of which corresponds to a type of atom that may be present in the input molecule. A “type of atom” may refer to an individual chemical element that the atom is. The voxelized representation may comprise multiple channels, one for each type of atoms or at least each type of heavy atoms present in the molecule. For instance, in some cases, the voxelized representation of a molecule (e.g. the input molecule) may include a first channel corresponding to a first atom type (e.g., carbon (C) atoms) that may be present in the input molecule and a second channel corresponding to a second atom type (e.g., nitrogen (N) atoms) that may be present in the input molecule. Each voxel in the first channel may be associated with a value indicative of the density of atoms of the first atom type at the corresponding location while each voxel in the second channel may be associated with a value indicative of the density of atoms of the second atom type at the corresponding location. Accordingly, as described in more details below, the molecule design computation model may denoise the voxelized representation of the input molecule by at least updating, for example, over multiple sampling iterations, the atomic density of one or more voxels in at least one channel of the voxelized representation of the input molecule. That is, in some cases, the term “denoising” refers to updating the voxelized representation of the input molecule, which may include updating the atomic density of at least one voxel in the voxelized representation of the input molecule. In some cases, updating the atomic density of a voxel in one channel of the voxelized representation of the input molecule may change the likelihood of the voxel being a portion of a type of atom associated with that channel.

In some example embodiments, the molecule design computation model may denoise the input molecule (e.g., the voxelized representation of the input molecule) over multiple sampling iterations, with each sampling iteration generating an updated voxelized representation that differs from the voxelized representation of the input molecule. In some cases, each updated voxelized representation may comprise a sample selected from a data distribution of molecules (e.g., voxelized representations of molecules) exhibiting one or more desired properties. In this context, the term “data distribution” may refer to the population of different molecular compositions and conformations (or three-dimensional structures). Those molecules that exhibit the one or more desired properties may congregate in higher density regions of the data distribution, meaning that the molecule design computation model should sample each updated voxelized representation from the higher density regions of the data distribution. However, this data distribution may be too high-dimensional to be approximated directly. For example, computing a probability density function (PDF) characterizing the probabilities of different molecules in the data distribution requires a normalizing constant. In the case of molecule design, this normalizing constant may correspond to the total quantity of molecules in the data distribution, which may be unfeasible to estimate. As such, in some cases, the molecule design computation model may be trained to approximate the data distribution by determining a function, such as a score function, that estimates the gradient (or change in densities) across the data distribution. As described in more details below, the molecule design computation model may use the function to guide the sampling of updated voxelized representations from the data distribution such that each successive sample is selected from incrementally higher density regions of the data distribution.

As noted, in some cases, denoising the input molecule over multiple sampling iterations, which includes making successive updates to the voxelized representation of the input molecule, may be tantamount to selecting successive samples from the data distribution of molecules (e.g., data distribution of the voxelized representations of molecules), with each sample corresponding to an updated voxelized representation that differs from the voxelized representation of the input molecule. For example, in some cases, the voxelized representation of the input molecule may be denoised by at least updating the atomic density of one or more voxels in at least one channel of the voxel grid forming the voxelized representation of the input molecule. In some cases, the molecules in the higher density regions of data distribution may exhibit one or more desired properties including, for example, drug-like properties such as affinity, specificity, biological activity, developability, and/or the like. Operating on the three-dimensional representation of the input molecule, such as a voxelized representation of the input molecule, may increase the likelihood that the conformation (or three-dimensional structure) of the resultant output molecule selected from the higher density regions of the data distribution and therefore exhibits the one or more desired properties.

In some cases, the molecule design computation model may be trained to approximate the data distribution, by training the molecule design computation model using training dataset of known molecules exhibiting the one or more desired properties (e.g., the PubChem dataset, the QM9 molecule dataset, the Geometric Ensemble of Molecules (GEOM) Drugs dataset, and/or the like). For example, in some cases, the molecule design computation model may be trained to approximate the data distribution by at least determining, for example, through Bayesian inference, a function (e.g., a score function and/or the like) approximating the different densities across the data distribution. In some cases, the function may be parametrized by the molecule design computation model, meaning that the parameters of the function (e.g., score function) are the parameters of the molecule design computation model, which were adjusted when the molecule design computation model is trained to approximate the data distribution. In some cases, the high density regions of the data distribution may be populated by molecules similar to the known molecules exhibiting the one or more desired properties whereas the low density regions of the data distribution may be populated by molecules dissimilar to the known molecules exhibiting the one or more desired properties. The score function of the data distribution may indicate the transitions between different density regions of the data distribution including, for example, transitions between higher density regions and lower density regions of the data distribution. As such, once trained, the molecule design computation model may sample the data distribution based on the score function such that each successive sample (or molecule) is selected from incrementally higher density regions of the data distribution.

In some example embodiments, the molecule design computation model may be trained to denoise a corrupted three-dimensional representation of a known molecule from the training dataset and recover the original three-dimensional representation of the known molecule. For example, in some cases, the corrupted three-dimensional representation of the known molecule may be generated by corrupting the three-dimensional representation of the known molecule with noise (e.g., Gaussian noise such as isotropic Gaussian noise). The training of the molecule design computation model may include adjusting one or more parameters (e.g., weights, biases, and/or the like) of the molecule design computation model to reduce (or minimize) a difference (e.g., mean squared error (MSE)) between the recovered three-dimensional representation of the known molecule and the original three-dimensional representation of the known molecule.

In some example embodiments, to avoid overfitting the molecule design computation model to the known molecules in the training dataset, the molecule design computation model may be trained to recover noisy versions of the three-dimensional representations of the known molecules in the training dataset instead of the original three-dimensional representations. That is, the three-dimensional representation of each known molecule in the training dataset may be adulterated with additional noise but this noise is not to be conflated with the noise that the molecule design computation model is trained to remove from the corrupted three-dimensional representation of each known molecule in the training dataset. In other words, in some cases, the molecule design computation model may be trained based on a training dataset that includes noisy three-dimensional representations of known molecules, and corrupted versions thereof. As described in more details below, in some cases, the noisy three-dimensional representation of a known molecule may be generated by adulterating the three-dimensional representation (e.g., voxelized representation) of the known molecule with a first quantity of noise (e.g., Gaussian noise such as isotropic Gaussian noise and/or the like) to smooth the density of the data distribution of the known molecules while still preserving at least a portion of the conformation (e.g., three-dimensional structure) of the known molecule, thereby obtaining a noisy representation of the known molecule. The noisy three-dimensional representation of the known molecule may then be further corrupted with a second quantity of noise (e.g., Gaussian noise such as isotropic Gaussian noise and/or the like) to generate the corrupted three-dimensional representation. In some cases, the molecule design computation model may be trained to denoise the corrupted three-dimensional representation of the known molecule, for example, by removing the second quantity of noise, and recover the noisy three-dimensional representation of the known molecule (which still includes the first quantity of noise). Moreover, in some cases, the training of the molecule design computation model may include gradient based Markov Chain Monte Carlo (MCMC) sampling (e.g., Langevin Markov Chain Monte Carlo (MCMC) sampling and/or the like) in which the parameters of the molecule design computation model are adjusted over successive sampling iterations to increase the similarity (e.g., reduce the mean squared error (MSE)) between the three-dimensional representation of each known molecule recovered by the molecule design computation model from the corresponding corrupted three-dimensional representation of the known molecule in the training dataset and the noisy three dimensional representation of the sample molecule in the training dataset. The score function derived in this manner may capture a data distribution with smoother density transitions, which mitigates the phenomenon of mode collapse where the molecule design computation model is less robust and capable of generating only a limited selection of output molecules (e.g., those within the immediate vicinity of the known molecules in the data distribution).

As described in more details below, during inference, the trained molecule design computation model may be applied to generate one or more output molecules by denoising the three-dimensional representation of an input molecule. In some cases, the input molecule may be a random molecule (e.g., a molecule with a random selection of atomic types and/or positions) or a known molecule having one or more undesirable properties, meaning that the three-dimensional representation of the input molecule may include at least some noise that require removal such that the three-dimensional representation of the output molecule generated therefrom is consistent with those exhibiting one or more desired properties. The molecule design computation model may do so by traversing the smoothed density of the noisy data distribution of noisy three-dimensional representations of molecules, for example, through one or more iterations of gradient-based Markov Chain Monte Carlo (MCMC) sampling (e.g., Langevin Markov Chain Monte Carlo and/or the like) towards incrementally higher density regions of the data distribution. Each iteration of gradient-based Markov Chain Monte Carlo (MCMC) sampling may include updating the three-dimensional representation of the input molecule, which is tantamount to selecting, from a different location in the noisy data distribution, the noisy three-dimensional representation of one or more molecules. Albeit selected from the noisy data distribution, the molecules corresponding to these noisy three-dimensional representations may be less distorted than the original three-dimensional representation of the input molecule. Such molecules corresponding to these noisy three-dimensional representations may be more consistent with molecules exhibiting the one or more desired properties, than the input molecule. In some cases, the noisy three-dimensional representations of the molecules selected from the noisy data distribution may undergo further denoising in order to recover the corresponding molecule by mapping the noisy three-dimensional representation of each molecule from the noisy data distribution to a corresponding clean three-dimensional representation of the molecule in the true data distribution of molecules exhibiting the one or more desired properties. It should be appreciated that sampling from the noisy data distribution may provide a number of advantages over sampling from the true data distribution of molecule exhibiting the one or more desired properties. For example, sampling from the noisy data distribution of molecules exhibiting the one or more desired properties, which exhibits smoother density transitions, may be less susceptible to mode collapse than sampling from the true data distribution. In some cases, this may be because there are fewer steep gradients or less drastic gradients in the noisy data distribution than in the true data distribution, where in the true data distribution steep gradients restrict sampling to the immediate vicinity of the known molecules characterizing the true data distribution. In other words, whereas the molecule design computation model may be capable of generating outputs with limited variety when sampling from the true data distribution (e.g., the aforementioned phenomenon called “mode collapse”), sampling from the noisy distribution may increase the variety of the model's outputs. Moreover, sampling from a noisy data distribution populated by noisy voxelized representations of molecules may provide additional advantages over sampling from a noisy data distribution populated by noisy conventional three-dimensional representations of molecules, such as point-cloud representations of molecules. For instance, in some cases, the molecule design computation model may be trained to operate on voxelized molecule representations and generate large, drug-like molecules with greater ease, effectiveness, expressiveness, and scalability. Unlike when operating on conventional three-dimensional molecule representations (e.g., point-cloud representations), operating on voxelized molecule representations may allow the disclosed molecule design computation model to function without having to specify the number of atoms present in the output molecule and without workarounds to reconcile the discrete distribution for atom types and continuous distribution for atomic position associated with each molecule.

Despite the aforementioned advantages, operating on voxelized molecule representations may impose a significant computational burden, which can scale exponentially with molecule size (e.g., number of constituent atoms). For example, while a small molecule containing 10 heavy atoms already requires a [32×32×32] voxel grid with 32,000 features (or atomic density values) per molecule, larger, more realistic drug-like molecules may require of a voxel grid of at least double that size with an exponentially greater quantity of points (e.g., a [64×64×64] voxel grid with 260,000 features (or atomic density values) per molecule). In some cases, applying the molecule design computation model to operate on voxelized representations of larger, more realistic drug-like molecules may be an intractable task as is the case with training the molecule design computation model on large training datasets (e.g., training datasets containing millions of voxelized representations of known molecules) to learn a more diverse molecular space (or chemical space). Furthermore, in practice, a large proportion of candidate molecules generated by the molecule design computation model may fail to be successfully synthesized in the laboratory, even in cases where the candidate molecules are realistic and valid. It may therefore be desired for the molecule design computation model to be applied to generate tens of thousands or even millions of candidate molecules. The computational burden associated with generating molecules, particularly larger molecules or larger quantities of molecules, may be reduced by the molecule design computation model operating on lower dimensional embeddings of voxelized molecule representations. For instance, in some cases, the molecule design computation model may be trained to generate an output molecule by denoising the embedding of the three-dimensional representation of an input molecule. As described in more details below, in some cases, the molecule design computation model may be trained based on a training dataset that includes the corrupted embeddings of sample molecules exhibiting one or more desired properties, each of which being generated by encoding the noisy three-dimensional representation (e.g., voxelized representation) of a known molecule exhibiting the one or more desired properties before the resulting embedding is corrupted with the addition of noise (e.g., Gaussian noise such as isotropic Gaussian noise and/or the like).

In some example embodiments, encoding the three-dimensional representation of a molecule, such as the voxelized representation of the molecule, may project the three-dimensional representation of the molecule (e.g. the voxelized molecule representation) from a high-dimensional discrete space populated by three-dimensional representations of molecules (e.g., a discrete voxelized space populated by voxelized molecule representations) into a lower-dimensional representation in a lower dimensional latent space populated by the corresponding molecule embeddings. In other words, encoding the three-dimensional representation of a molecule, such as the voxelized representation of the molecule, may take as input a three-dimensional representation of the molecule (e.g. the voxelized molecule representation) in a high-dimensional discrete space populated and produce as output a lower-dimensional representation of the molecule (i.e. a molecule embedding corresponding to the input three-dimensional representation) in a lower dimensional latent space. Encoding the three-dimensional representation of a molecule, such as the voxelized representation of the molecule, may be performed using a machine learning model that has been trained to identify a latent space representation of a three-dimensional representation of input molecule from which the three-dimensional representation of the input molecule can be recovered. In some cases, each embedding in the latent space may be a latent space representation of the voxelized representation of a corresponding molecule. Accordingly, the embedding of the voxelized representation of the molecule may have a different dimensionality, or quantity of features, than the voxelized representation of the molecule. For example, in some cases, encoding the voxelized representation of the molecule may reduce the dimensionality or the quantity of features present in the voxelized representation of the molecule. As such, the computational burden of denoising the voxelized representation of the molecule to generate one or more output molecules therefrom may be reduced by the molecule design computation model operating on the embedding of the voxelized representation of the molecule instead of the on the voxelized representation of the molecule directly at least because the embedding contains a fewer quantity of features. Furthermore, in some cases, the molecule design computation model may be trained to approximate a noisy data distribution of molecules exhibiting one or more desired properties such that the one or more output molecules may be generated by sampling therefrom. This noisy data distribution, which may be populated by noisy embeddings of the voxelized representations of molecules, may exhibit smoother density transitions than the corresponding true data distribution. As such, the noisy data distribution may support more efficient sampling (e.g., via gradient-based Markov Chain Monte Carlo (MCMC) sampling such as Langevin Markov Chain Monte Carlo and/or the like) at least because the noisy data distribution may exhibit fewer of the steep gradient changes that would prevent the molecule design computation model from adequately exploring the data distribution when sampling therefrom.

FIGS. 1A-1B depict system diagrams illustrating different examples of a molecule design system 100, in accordance with some example embodiments. Referring to FIGS. 1A-1B, in some cases, the molecule design system 100 may include a molecule design engine 110, a training engine 120, and a client device 130. In the examples of the molecule design system 100 shown in FIGS. 1A-1B, the molecule design engine 110, the training engine 120, and the client device 130 may be communicatively coupled via a network 140. The client device 130 may be a processor-based device including, for example, a workstation, a desktop computer, a laptop computer, a smartphone, a tablet computer, a wearable apparatus, and/or the like. The network 140 may be a wired network and/or a wireless network including, for example, a local area network (LAN), a virtual local area network (VLAN), a wide area network (WAN), a public land mobile network (PLMN), the Internet, and/or the like. In the examples shown in FIGS. 1A-1B, the molecule design computation model 115 may include a denoising model 117 trained to generate the output molecule 162 by at least denoising the input molecule 152. The denoising model is a machine learning model trained to take as input a corrupted three dimensional representation of a molecule or a lower dimensional embedding thereof, wherein a corrupted three dimensional representation of a molecule is a three dimensional representation of a molecule to which noise has been added, and produce as output a corresponding denoised three dimensional representation of the molecule or lower dimensional embedding thereof, using training data comprising three dimensional representations of a plurality of known molecules and corresponding corrupted three dimensional representations. The known molecules in the training data may comprise a plurality of molecules exhibiting one or more desired properties. The denoising model may be an artificial neural network (ANN). The denoising model may be a deep learning model. The denoising model may be an encoder-decoder three-dimensional convolutional neural network (CNN). For example, in some cases, the molecule design computation model 115 may apply the denoising model 117 to denoise the three-dimensional representation of the input molecule 152 or, alternatively, the embedding 154 of the three-dimensional representation of the input molecule 152, in order to generate the output molecule 162. In some cases, the denoising model 117 may denoise the three-dimensional representation of the input molecule 152 (or the embedding 154 thereof) over multiple successive sampling iterations, with a portion of the noise present in the three-dimensional representation of the input molecule 152 (or the embedding 154 thereof) being removed at each sampling iteration.

As described in more details below, the denoising of the three-dimensional representation of the input molecule 152 may alter the composition and/or the conformation (or three-dimensional structure) of the input molecule 152 such that the composition and the conformation (or three-dimensional structure) of the resulting output molecule 162 are consistent with those of molecules exhibiting one or more desired properties. In instances where the output molecule 162 is a drug molecule, for example, the one or more desired properties may include drug-like properties such as affinity, specificity, biological activity, and developability. In some cases, whether the output molecule 162 exhibits certain desired properties may be contingent upon the output molecule 162 exhibiting a corresponding conformation (or three-dimensional structure). As such, in some cases, the molecule design computation model 115 applying the denoising model 117 to operate on the three-dimensional representation of the input molecule 152 (or the embedding 154 thereof) instead of a one-dimensional or two-dimensional representation of the input molecule 152 increases the likelihood that the resulting output molecule 162 exhibits a conformation (or three-dimensional structure) consistent with the one or more desired properties.

In some example embodiments, the molecule design computation model 115, including the denoising model 117, may be trained to learn or approximate the data distribution of molecules exhibiting the one or more desired properties (e.g., drug-like properties such as affinity, specificity, biological activity, and developability). For example, in some cases, the molecule design computation model 115 may be trained to approximate the data distribution of molecules exhibiting the one or more desired properties based on a training dataset of known molecules that exhibit the one or more desired properties (e.g., the PubChem dataset, the QM9 molecule dataset, the Geometric Ensemble of Molecules (GEOM) Drugs dataset, and/or the like). As described in more details below, the molecule design computation model 115 may be trained to approximate a noisy data distribution, which is populated by noisy three-dimensional representations of the known molecules exhibiting the one or more desired properties. Moreover, the molecule design computation model 115 may be trained to operate in either a discrete space populated by the three-dimensional representations (e.g., voxelized representation) of the molecules exhibiting the one or more desired properties or, alternatively, in a latent space that is populated by embeddings of the three-dimensional representations (e.g., embeddings of the voxelized representations) of the molecules, which are lower-dimensional representations of the molecules.

To further illustrate, FIG. 1A depicts an example of the molecule design engine 110 of FIG. 1A in which the molecule design computation model 115 is trained to operate in a discrete space (three-dimensional voxelized representation) while the molecule design computation model 115 included in the example of the molecule design engine 110 shown in FIG. 1B may be trained to operate in a latent space. The latent space may comprise continuous values (embeddings) for a plurality of features. In either example of the molecule design engine 110, the molecule design computation model 115 may be trained to approximate a noisy data distribution, for example, by being trained based on noisy three-dimensional representations of the molecules exhibiting the one or more desired properties. For example, the example of the molecule design computation model 115 shown in FIG. 1A may be trained to approximate a noisy discrete distribution populated by noisy three-dimensional representations of the molecules whereas the example of the molecule design computation model 115 shown in FIG. 1B may be trained to approximate a noisy latent distribution populated by noisy embeddings of the three-dimensional representations of the molecules.

Referring first to FIG. 1A, in some cases, the molecule design engine 110 may include the molecule design computation model 115 and a recovery model 118. Alternatively, FIG. 1B depicts another example of the molecule design engine 110 that may also include, in addition to the molecule design computation model 115 and the recovery model 118, an encoder 111 and a decoder 119. In some example embodiments, the molecule design engine 110 may apply the molecule design computation model 115 to generate, based at least on an input molecule 152, an output molecule 162. For instance, in the example of the molecule design engine 110 shown in FIG. 1A, the molecule design computation model 115 may operate on a three-dimensional representation of the input molecule 152 which, in some cases, may be a voxelized representation of the input molecule 152. In doing so, the molecule design computation model 115 may operate in a discrete voxelized space populated by noisy voxelized representations of different molecules (e.g., molecules exhibiting the one or more desired properties). Alternatively, in the example of the molecule design engine 110 shown in FIG. 1B, the molecule design computation model 115 may operate on the embedding 154 of the input molecule 152. As described in more details below, the embedding 154 of the input molecule 152 may be generated by the encoder 111 encoding the three-dimensional representation (e.g., voxelized representation) of the input molecule 152. In this variation of the molecule design engine 110, the molecule design computation model 115 may operate in a latent space that is populated by noisy embeddings of the three-dimensional representations (e.g., voxelized representations) of different molecules.

In some example embodiments, the molecule design computation model 115 may include a denoising model 117 trained to denoise, based on a function 175, the three-dimensional representation of the input molecule 152 such that the resultant three-dimensional representation of the output molecule 162 is sampled from a higher density region of the data distribution of molecules exhibiting the one or more desired properties. FIG. 1A depicts one example of the denoising model 117 that is trained to denoise the three-dimensional representation of the input molecule 152 while FIG. 1B depicts another example of the denoising model 117 trained to denoise the embedding 154 of the three-dimensional representation of the input molecule 152. In some cases, the denoising model 117 may denoise the three-dimensional representation of the input molecule 152 (e.g., the voxelized representation of the input molecule 152) or, alternatively, the embedding 154 thereof, over multiple timesteps. In some cases, the denoising performed at each timestep may be tantamount to selecting one or more samples (e.g., intermediate molecules) from different locations in the data distribution. In some cases, the function 175 may be a score function that outputs a value (e.g., a score) indicative of the local density change at a particular location within the data distribution (e.g., a location occupied by a certain molecule). Accordingly, the denoising of the input molecule 152 may be performed based on the output of the score function such that each successive sample (or molecule) is selected from an incrementally higher density region of the data distribution.

In some example embodiments, the denoising of the input molecule 152 may include updating the three-dimensional representation (e.g., voxelized representation) of the input molecule 152 (or the embedding 154 thereof), which may be representative of the composition and the conformation (or three-dimensional structure) of the input molecule 152, to increase the likelihood of the resulting output molecule 162 being in the data distribution of molecules exhibiting the one or more desired properties. In some cases, the molecule design computation model 115 may apply the denoising model 117 to modify the three-dimensional representation of the input molecule 152 (or the embedding 154 thereof) over one or more iterations of gradient-based Markov Chain Monte Carlo (MCMC) sampling (e.g., Markov Chain Monte Carlo (MCMC) with Langevin dynamics and/or the like). For example, in some cases, each iteration of gradient-based Markov Chain Monte Carlo (MCMC) sampling may include selecting, from the data distribution, a sample (or a molecule) that includes one or more modifications to the three-dimensional representation of the input molecule 152 (or the embedding 154 thereof). As noted, in some cases, the sampling from the data distribution may be guided by the function 175 (e.g., score function). For instance, in cases where the function 175 is a score function, the function 175 may output, for each sample (or molecule) selected from the data distribution, a value (e.g., a score) corresponding to the change in density observed at the location in the data distribution occupied by the sample (or molecule). As such, in some cases, the sampling from the data distribution may be guided by the function 175 such that each successive sample is selected from incrementally higher density regions of the data distribution.

To further illustrate, in some cases, the denoising model 117 may be applied update the three-dimensional representation (e.g., voxelized representation) of the input molecule 152 (or the embedding 154 thereof) by at least selecting, for example, a first sample and a second sample from the data distribution. It should be appreciated that each of the first sample and the second sample may correspond to a modified three-dimensional representation of the input molecule 152 in the example shown in FIG. 1A or, in the case of FIG. 1B, a modified embedding of the three-dimensional representation the input molecule 152. In cases where the function 175 is a score function, the function 175 may assign a first value (e.g., a first score) to the first sample to indicate a more positive local change (e.g., an increase or a smaller decrease) in the density of the data distribution at a first location of the first sample and a second value to the second sample to indicate a less positive local change (e.g., a smaller increase or a decrease) in the density of the data distribution at a second location of the second sample. In some cases, the molecule design computation model 115 may apply the denoising model 117 to select a third sample (e.g., another modified three-dimensional representation or another modified embedding) from the data distribution by further modifying the first sample in order to sample the third sample from a higher density region of the data distribution than the first sample and the second sample.

In some example embodiments, the molecule design computation model 115 may apply the denoising model 117 to denoise a voxelized representation of the input molecule 152 (or the embedding 154 thereof) instead of a conventional three-dimensional representation of the input molecule 152, such as a point-cloud representation of the input molecule 152 and/or the like. For example, in some cases, the voxelized representation of the input molecule 152 may represent the types of atoms and the positions of atoms present in the input molecule 152 as continuous (e.g., Gaussian-like) densities across a three-dimensional voxel grid. To indicate the positions of the atoms present in the input molecule 152, each voxel in the voxel grid may be associated with a value indicative of the atomic density at the corresponding location. In some cases, the atomic density associated with a particular voxel in the voxel grid may correspond to the likelihood of that voxel being a portion of an atom at that location. For instance, a first voxel having a higher atomic density may be more likely to be a portion of an atom forming the input molecule 152 than a second voxel having a lower atomic density. Accordingly, the voxelized representation of the input molecule 152 may represent the positions of the atoms the input molecule 152 differentiating, based on the atomic density associated with each voxel in the voxel grid, between the voxels in the voxel grid that form a portion of an atom in the input molecule 152 and the voxels in the voxel grid that do not form a portion of an atom in the input molecule 152. In some cases, the atoms forming the input molecule 152 may be disposed at the locations of those voxels associated with an atomic density that satisfies one or more thresholds.

In some example embodiments, the voxelized representation of the input molecule 152 may include one or more channels, each of which corresponding to a type of atoms that may be present in the input molecule 152. For example, in some cases, the voxelized representation of the input molecule 152 may include a separate channel for each type of heavy atom that may be present in the input molecule 152. That the voxelized representation of the input molecule 152 includes different channels for different types of atoms may obviate the discrete distribution typically associated with atom types found in conventional three-dimensional representations (e.g., point-cloud representation and/or the like). Instead, the voxelized representation of the input molecule 152 may represent the types and positions of the atoms in the input molecule 152 as one or more continuous (e.g., Gaussian-like) densities across the aforementioned three-dimensional voxel grid. For instance, the voxelized representation of the input molecule 152 may include a first channel representative of a first type of atoms (e.g., carbon @ atoms) that may be present in the input molecule 152. The presence of the first type of atoms in the input molecule 152 and their respective locations may represented by a first continuous (e.g., Gaussian-like) density across the first channel in the voxelized representation of the input molecule 152. Note that the density is continuous in the sense that the value associated with each voxel can take a continuous value (e.g. a value within a continuous, bounded or unbounded distribution). In some cases, the voxelized representation of the input molecule 152 may further include a second channel representative of a second type of atoms (e.g., nitrogen (N) atoms) that may be present in the input molecule 152. The presence of the second type of atoms and their respective locations may be represented by a second continuous (e.g., Gaussian-like) density across the second channel in the voxelized representation of the input molecule.

Unlike conventional three-dimensional representations of the input molecule 152 (e.g., point-cloud representation and/or the like) that represents the types and the positions of the atoms in the input molecule 152 as two separate types of distributions (e.g., discrete distribution for atom types and continuous distribution for atomic position), the voxelized representation of the input molecule 152 may jointly represent the types and positions of the atoms in the input molecule 152 as one or more continuous (e.g., Gaussian-like) distributions in the manner described above. As such, the molecule design computation model 115 may apply the denoising model 117 to operate on the voxelized representation of the input molecule 152 without workarounds to reconcile two different types of distributions, which are necessary with conventional three-dimensional representations of the input molecule 152. The voxelized representation of the input molecule 152 may also be more representative of the conformation (or three-dimensional structure) of the input molecule 152 than conventional three-dimensional representations of the input molecule 152. For example, the voxelized representation of the input molecule 152 may capture long-range dependencies between distant atoms, even in instances where the input molecule 152 contains a large quantity of atoms. Furthermore, the molecule design computation model 115 may apply the denoising model 117 to denoise the voxelized representation of the input molecule 152 and generate the output molecule 162 without any a priori knowledge of the quantity of molecules present in the output molecule 162.

In some example embodiments, the training engine 120 may be trained to generate, for inclusion in a training dataset, one or more training samples. FIG. 1A depicts one example of the training engine 120 in which a corruption engine 121 generates each training sample by adding noise (e.g., Gaussian noise such as isotropic Gaussian noise) to a noisy three-dimensional representation 182 of a sample molecule (e.g., known molecule exhibiting one or more desired properties), thereby generating a corrupted three-dimensional representation 184 of the sample molecule. It should be appreciated that the corruption engine 121 may add noise to an already noisy three-dimensional representation 182 of the sample molecule in order for the molecule design computation model 115 to be trained to approximate a noisy data distribution of molecules exhibiting the one or more desired properties, which has smoother density transitions than the true data distribution of molecules exhibiting the one or more properties. As such, in some cases, the molecule design computation model 115, including the denoising model 117, may be trained to denoise the corrupted three-dimensional representation of the sample molecule and recover, for each training sample, the corresponding noisy three-dimensional representation of the sample molecule and not the clean (or original) three-dimensional representation of the sample molecule. Doing so may be tantamount to sampling from the noisy data distribution of molecules exhibiting the one or more desired properties and not the true distribution of the molecules.

Alternatively, FIG. 1B depicts another example of the training engine 120 in which the encoder 111 first encodes the noisy three-dimensional representation 182 of the sample molecule to generate an embedding 186 thereof before the corruption engine 121 adds noise to generate a corrupted embedding 188 of the noisy three-dimensional representation 182 of the sample molecule. In this variation of the molecule design engine 110, the molecule design computation model 115 may be trained to approximate a noisy latent distribution of the noisy embeddings of the three-dimensional representations of molecules exhibiting the one or more desired properties instead of the noisy but discrete distribution that the molecule design computation model 115 is trained to approximate in the example shown in FIG. 1A. To achieve this result, the training engine 120 may generate each training sample in the training dataset to include a corrupted embedding 188. For example, FIG. 1B shows that the training engine may further include the encoder 111, which may first encode the noisy three-dimensional representation 182 of the sample molecule (e.g., known molecule exhibiting one or more desired properties) to generate the embedding 186 before the corruption engine 121 adds noise thereto in order to generate the corrupted embedding 188. In some cases, the molecule design computation model 115, including the denoising model 117, may be trained to denoise the corrupted embedding 188 and recover the embedding 186 therefrom. The denoising model 117, encoder 111 and corresponding decoder may be trained together.

In some example embodiments, the training of the molecule design computation model 115 may including adjusting one or more parameters (e.g., weights, biases, and/or the like) of the denoising model 117. In the example shown in FIG. 1A, the parameters of the denoising model 117 may be adjusted such that the denoising model 117 is able to recover, from the corrupted three-dimensional representation 184 of the sample molecule, the corresponding noisy three-dimensional representation 182 of the sample molecule. For example, as described in more details below, the one or more parameters of the denoising model 117 may be adjusted, over multiple iterations, to increase (or maximize) the similarity (e.g., reduce (or minimize) the mean squared error (MSE)) between the noisy three-dimensional representation 182 of the sample molecule recovered by the denoising model 117 from the corrupted three-dimensional representation 184 of the sample molecule and the original noisy three-dimensional representation 182 of the sample molecule. Alternatively, the example in FIG. 1B shows that the parameters of the denoising model 117 may be adjusted such that the denoising model 117 is able to recover the embedding 186 that is generated by encoding the noisy three-dimensional representation 182 of the sample molecule from the corrupted embedding 188. For instance, in the example shown in FIG. 1B, the parameters of the denoising model 117 may be adjusted to increase (or maximize) the similarity (e.g., reduce (or minimize) any loss function that quantifies the difference between the embedding 186 recovered by the denoising model 117 from the corrupted embedding 188 and the original, uncorrupted embedding 186 of the three-dimensional representation 182 of the sample molecule, e.g. the mean squared error (MSE)) between the embedding 186 recovered by the denoising model 117 from the corrupted embedding 188 and the original, uncorrupted embedding 186 of the three-dimensional representation 182 of the sample molecule.

In some example embodiments, the training of the molecule design computation model 115 may include determining the function 175, which may be parameterized by the parameters (e.g., weights, biases, and/or the like) of the denoising model 117. For example, in some cases, the training of the molecule design computation model 115, which includes adjusting the one or more parameters of the denoising model 117, may determine the function 175 (e.g., score function) by at least adjusting the corresponding parameters of the function 175. In some cases, the function 175 may approximate the different densities across the data distribution of molecules exhibiting the one or more desired properties, with molecules exhibiting the one or more properties being more likely to occupy higher density regions of the data distribution. In the example shown in FIG. 1A, this data distribution may be a noisy data distribution populated by noisy three-dimensional representations of molecules. Alternatively, in the example of the molecule design engine 110 shown in FIG. 1B, this data distribution may be a noisy latent distribution populated by noisy embeddings of the three-dimensional representations of molecules.

As noted, in some example embodiments, overfitting of the molecule design computation model 115, including the denoising model 117, to the known molecules in the training dataset may be avoided by training the denoising model 117 to approximate a noisy data distribution populated by noisy three-dimensional representations of molecules, such as a noisy voxelized representations. training dataset. One example of this is shown in FIG. 1A where the molecule design computation model 115 is trained to recover the noisy three-dimensional representation 182 of the sample molecule and not the clean (or original) three-dimensional representation of the sample molecule. As described in more details below, once trained, the molecule design computation model 115 may generate the three-dimensional representation of the output molecule 158 by traversing (i.e. iteratively sampling different regions of) the smoothed densities of the noisy data distribution to sample at least one updated three-dimensional representation 160. The noisy data distribution may be populated by noisy three-dimensional representations of molecules exhibiting the one or more desired properties. As such, the updated three-dimensional representation 160 may include at least some noise that may be removed by applying the recovery model 118 to denoise the updated three-dimensional representation 160. The recovery model 118 may be a machine learning model that has been trained to take as input a noisy three-dimensional representation 160 of a molecule and produce as output a corresponding denoised three-dimensional representation. Doing so may generate the three-dimensional representation of the output molecule 152, which occupies the true data distribution of the clean three-dimensional representations of molecules exhibiting the one or more desired properties.

Alternatively, FIG. 1B depicts another example of the molecule design engine 110 in which the molecule design computation model 115 is trained to approximate a noisy latent distribution populated by embeddings of the noisy three-dimensional representations of molecules exhibiting the one or more desired properties. In this variation of the molecule design computation model 115, the denoising engine 117 may be trained to recover the embedding 186 of the noisy three-dimensional representation 182 of the sample molecule from the corrupted embedding 188. Once trained, the molecule design computation model 115 may apply the denoising model 117 to denoise the embedding 154 generated by the encoder 111 encoding the three-dimensional representation of the input molecule 152. The denoising may include the denoising model 117 updating the embedding 154 over multiple successive sampling iterations to generate at least one updated embedding 156 during each sampling iteration. Doing so may be tantamount to the molecule design computation model 115 selecting samples from the noisy latent distribution and the molecule design computation model 115 may continue to select samples therefrom until one or more criteria are met. In some cases, the updated embedding 156 may be decoded, for example, by the decoder 119, before the resulting noisy three-dimensional representation 158 is denoised by the recovery model 118 to generate the three-dimensional representation of the output molecule 162. As described in more details below, in this example of the molecule design engine 110, the molecule design computation 115 may operate in a noisy latent space populated by noisy embeddings of the three-dimensional representations of molecules and not the noisy three-dimensional representations found in the discrete voxelized space. Moreover, it should be appreciated that although the denoising model 117 and the recovery model 118 may, in some cases, share the same architecture (e.g., artificial neural network (ANN) and/or the like), the two models are trained to remove different noise. For example, the denoising model 117 may be trained to denoise the three-dimensional representation of the input molecule 152 or the embedding 154 thereof such that the resultant three-dimensional representation of the output molecule 162 is consistent with the composition and/or conformation of molecules exhibiting the one or more desired properties (e.g., drug-like properties). Contrastingly, the recovery model 118 may be trained to remove the noise that is added to smooth the densities of the known molecules available to train the molecule design computation model 115.

Referring again to FIG. 1B, the embedding 154 of the three-dimensional representation of the input molecule 152 may be generated by the encoder 111 encoding the three-dimensional representation of the input molecule 152. In some cases, the embedding 154 may be a lower-dimensional representation of the three-dimensional representation of the input molecule 152 generated by the encoder 111 reducing the dimensionality of the three-dimensional representation of the input molecule 152. For example, in some cases, the encoder 111 and the decoder 119 may form an autoencoder including, for example, a variational autoencoder (VAE) such as a vector quantized variational autoencoder (VQ-VAE) and/or the like. The encoder 111 may generate the embedding 154 by at least reducing the dimensionality of the three-dimensional representation (e.g., voxelized representation) of the input molecule 152. In this context, reducing the dimensionality of the three-dimensional representation of the input molecule 152 may include down sampling, compressing, or reducing the dimensionality the three-dimensional representation of the input molecule 152, for example, by condensing at least some of the features (e.g., atomic density values) present in the three-dimensional representation of the input molecule 152 such that the resulting embedding 154 includes a fewer quantity of features than the original three-dimensional representation of the input molecule 152 but those features still capture the same (or similar) information conveyed in the original three-dimensional representation of the input molecule 152. For a voxelized representation of the input molecule 152, each feature present therein may correspond to the atomic density value associated with each voxel included in the voxel grid representative of the input molecule 152. For instance, where the voxelized representation of the input molecule 152 includes a [32×32×32] voxel grid, the voxelized representation of the input molecule 152 may include 32,000 features (or atomic density values). At least some of those 32,000 features may be condensed by the encoder 111 when generating the embedding 154. In doing so, the embedding 154 may include fewer features, such as 4×4×4=64 features, for the denoising model 117 to operate upon when generating the output molecule 162.

As noted, the embedding 154 of the three-dimensional representation of the input molecule 152 shown in FIG. 1B may include a fewer quantity of features than the original three-dimensional representation of the input molecule 152. Moreover, the embedding 154 may be generated by the encoder 111 down sampling, compressing, or reducing the dimensionality of the three-dimensional representation of the input molecule 152. Doing so may be tantamount to the encoder 111 mapping the three-dimensional representation of the input molecule 152 from a high dimensional discrete voxelized space to a lower dimensional latent space. In some cases, the molecule design computation model 115 (e.g., the denoising engine 117) may denoise the embedding 154, for example, over one or more iterations of gradient-based Markov Chain Monte Carlo (MCMC) sampling. During each iteration of gradient based Markov Monte Carlo (MCMC) sampling, the molecule design computation model 115 (e.g., the denoising model 117) may sample at least one updated embedding 156 from this lower dimensional latent space. That the embedding 154 may include orders of magnitude fewer features than the original three-dimensional representation of the input molecule 152 means that the molecule design computation model 115 may apply the denoising model 117 to operate on the embedding 154 and generate the output molecule 162 faster and with greater computational efficiency while achieving comparable or better performance both qualitatively and quantitatively. This may be particularly advantageous in applications, such as computational drug design, that require generating a large quantity of candidate molecules in a short period of time. In some cases, reducing the dimensionality of the original three-dimensional representation of the input molecule 152 may enable the denoising model 117 to operate on and generate larger scale molecules (e.g., molecules containing upwards of 200 atoms) as well as larger quantities of molecules.

In some cases, the compactness of the embedding 154 relative to the three-dimensional representation of the input molecule 152 also means that less computational resources are necessary when operating on the embedding 154. For example, in order for the denoising model 117 to operate directly on the three-dimensional representation of the input molecule 152 (e.g., [32×32×32] voxel grid with 32,000 features), the denoising model 117 may be implemented to include a large number of trainable parameters (e.g., 100 million parameters). Contrastingly, the denoising model 117 may be realized with far fewer trainable parameters if the denoising model 117 is applied to operate on the embedding 154 instead. Implementing the denoising model 117 with fewer parameters may improve the performance of the denoising model 117 as a larger quantity of parameters may reduce the generalizability of the denoising model 117. For instance, the denoising model 117 may be especially prone to overfitting in instances where the denoising model 117 includes a large quantity of feature but relatively few known molecules are available to train the denoising model 117. When the denoising model 117 is overfitted to the known molecules in the training dataset, meaning that the denoising model 117 is trained too well on the training dataset (i.e. trained to learn a data distribution that is too tightly centered around the molecules in the training dataset), the denoising model 117 may be unable to generalize. Generalization in this context refers to the ability to accurately denoise the input molecule 152 if the input molecule 152 that is not one of the known molecules in the training dataset. As such, overfitting the denoising model 117 may prevent the denoising model 117 from accurately denoising any molecule that is not one of the known molecules in the training data.

Referring again to FIG. 1B, in the example of the molecule design engine 110 shown therein, the updated embedding 156 generated by sampling from the latent voxelized space may be decoded by the decoder 119 before the resulting noisy three-dimensional representation 158 is denoised by the recovery engine 118. While the decoding performed by the decoder 119 may map the updated embedding 156 from the latent space to the discrete space, the subsequent denoising performed by the recovery model 118 may constitute a jump from the noisy data distribution back to the true data distribution of molecules. For example, during each round of gradient-based Markov Chain Monte Carlo (e.g., Langevin Markov Chain Monte Carlo and/or the like), the molecule design computation model 115 may apply the denoising model 117 to sample from the noisy latent distribution of molecules exhibiting the one or more desired properties. Sampling from the noisy latent distribution in this context may include updating the embedding 154 of the three-dimensional representation (e.g., voxelized representation) of the input molecule 152. Doing so may be tantamount to selecting, from the data distribution, at least one updated embedding 156, which is then decoded by the decoder 119 before being denoised by the recovery model 118 to generate the three-dimensional representation (e.g., voxelized representation) of the output molecule 162. As described in more details below, the molecule design computation model 115 may further update the noisy embedding 156, for example, over multiple iterations of gradient based Markov Chain Monte Carlo (MCMC) sampling, until one or more criteria are met.

Once the one or more criteria are met, the molecule design engine 110 may recover, based at least on the three-dimensional representation of the output molecule 162, the output molecule 162. For instance, in some cases, the molecule design engine 110 may recover, based at least on the three-dimensional representation of the output molecule 162, the positions (e.g., coordinates) of the atoms present in the output molecule 162 and one or more bonds therebetween. Recovering the positions of the atoms present in the output molecule 162 may be performed by identifying maxima (e.g., peaks) of the predicted densities comprised in the three-dimensional representation of the output molecule 162. In doing so, the molecule design engine 110 may determine another representation of the output molecule 162 including, for example, a one-dimensional representation of the output molecule 162 (e.g., a simplified molecular-input line-entry system (SMILES) string), a two-dimensional representation of the output molecule 162 (e.g., a molecular graph), and/or the like. It should be appreciated that the output molecule 162 that is generated in this manner may be more likely to exhibit the one or more desired properties of the molecules in the data distribution. In particular, the denoising model 117 may generate the output molecule 152 to exhibit a composition and/or a conformation (or three-dimensional structure) that are consistent with the one or more desired properties. By operating on the embedding 154 of the three-dimensional representation (e.g., voxelized representation) of the input molecule 152, the denoising model 117 may generate the output molecule 162 faster and with less computational burden.

As noted, in some example embodiments, the molecule design computation model 115 may be trained to recover the noisy three-dimensional representation 182 of the sample molecule (e.g., a noisy voxelized representation of the sample molecule), which is generated by adding noise (e.g., Gaussian noise such as isotropic Gaussian noise and/or the like) to the three-dimensional representation of the sample molecules. In the example shown in FIG. 1A, the molecule design computation model 115 may be trained to denoise the corrupted three-dimensional representation 184 of the sample molecule by at least modifying the corrupted three-dimensional representation 184 of the sample molecule in order to recover the noisy three-dimensional representation 182 of the sample molecule. Alternatively, FIG. 1B shows an example of the molecule design engine 110 in which the molecule design computation model 115 is trained to recover the embedding 186 of the noisy three-dimensional representation 182 of the sample molecule. As shown in FIG. 1B, in some cases, the molecule design computation model 115 may denoise the corrupted embedding 188 by at least modifying the corrupted embedding 186, which may be generated by the corruption engine 121 adding noise (e.g., Gaussian noise and/or the like) to the embedding 184 of the three-dimensional representation 182 of the sample molecule. As noted, the embedding 184 may be generated by the encoder 111 down sampling or reducing the dimensionality of the noisy three-dimensional representation 182 (e.g., noisy voxelized representation) of the sample molecule. However, as noted, the down sampling of the noisy three-dimensional representation 182 (e.g., noisy voxelized representation) of the sample molecule may be optional, which may be the case when the encoder 111 implements an identity function. Thus, in some cases, it may be possible that the embedding 184 includes the same quantity of features as the original noisy three-dimensional representation 182 (e.g., noisy voxelized representation) of the sample molecule. In those instances, the embedding 154 may capture the same information present in the original three-dimensional representation (e.g., voxelized representation) of the input molecule 152 without condensation of the features present therein. In other words, the encoding of the three-dimensional representation (e.g., voxelized representation) of the input molecule 152 may be an optional operation, even with the inclusion of the encoder 111 in the example of the molecule design system 100 shown in FIG. 1B.

FIG. 2 depicts a flowchart illustrating an example of a process 200 for machine learning enabled generation of three-dimensional molecules in voxelized space, in accordance with some example embodiments. Referring to FIGS. 1A-1B and 2, the process 200 may be performed by the molecule design engine 110 to train and apply the molecule design computation model 115 to generate the output molecule 162 by at least denoising a three dimensional representation, such as a voxelized representation, of the input molecule 152. For example, in some cases, the molecule design computation model 115 may be trained on the noisy three-dimensional representation 182 of the sample molecule, which is generated by adding noise to the original three-dimensional representation of the sample molecule, such that the molecule design computation model 115 is trained to approximate a noisy data distribution with smoother density transitions. FIG. 1A shows one variation in which the molecule design computation model 115 is trained on the corrupted three-dimensional representation 184 of the sample molecule, which may be generated by adding additional noise to the noisy three-dimensional representation 182 of the sample molecule without any down sampling or compression. Alternatively, FIG. 1B shows another variation in which the molecule design computation model 115 is trained on the corrupted embedding 186 of the noisy three-dimensional representation 182 of the sample molecule. This corrupted embedding 188 may be generated by adding noise to the embedding 154 of the noisy three-dimensional representation 182 of the sample molecule, which is generated by the encoder 111 down sampling (or compressing) the features present in the noisy three-dimensional representation 182 (e.g., noisy voxelized representation) of the sample molecule. In other words, it should be appreciated that the molecule design computation model 115 may be trained to operate in either a noisy discrete voxelized space populated by noisy three-dimensional representations of molecules or, alternatively, in a noisy latent voxelized space populated by embeddings of the noisy three-dimensional representations of molecules.

At 202, the training engine 120 may generate a training dataset to include a plurality of corrupted sample molecules. In some example embodiments, generating a training dataset may include the training engine 120 generating a training dataset to include multiple corrupted sample molecules. This training dataset may then be used to train the molecule design computation model 115 to approximate a data distribution of molecules exhibiting one or more desired properties (e.g., drug-like properties). In some cases, each corrupted sample molecule may be a noisy three-dimensional representation of a known molecule that is further corrupted with additional noise (e.g., Gaussian noise such as isotropic Gaussian noise and/or the like). For example, FIG. 1A shows one example of this in which the corruption engine 121 generates the corrupted three-dimensional representation 184 of the sample molecule by adding additional noise to the noisy three-dimensional representation 182 of the sample molecule. As described in more details below, the molecule design computation model 115 (e.g., the denoising model 117) may be trained to approximate a noisy data distribution with smoother density transitions by being trained to recover the noisy three-dimensional representation 182 of the sample molecule from the corrupted three-dimensional representation 184 of the sample molecule. Alternatively, FIG. 1B shows another example in which the training engine 120 generates each corrupted sample molecule in the training dataset to include the corrupted embedding of the noisy three-dimensional representation 182 of the sample molecule. For instance, in some cases, the corruption engine 121 may generate the corrupted embedding 188 by adding noise (e.g., Gaussian noise such as isotropic Gaussian noise and/or the like) to the embedding 186 of the noisy three-dimensional representation 182 (e.g., noisy voxelized representation) of the sample molecule. In some cases, the training engine 120 may further augment the training dataset may be augmented by applying, to the noisy three-dimensional representation 182 (e.g., voxelized representation) of the sample molecule, one or more transformations including, for example, translations (e.g., by shifting the center of the sample molecule on each of three dimensions by sampling an uniform shift), rotations (e.g., by sampling three Euler angles uniformly), reflections, and/or the like.

Training the molecule design computation model 115 based on the noisy three-dimensional representation 182 of the sample molecule may mitigate the incidence of overfitting and mode collapse, which typically occur when the molecule design computation model 115 is trained to approximate a high-dimensional data distribution (e.g., the molecular space of 1060 possible chemical compounds) based on disproportionately few known molecules (e.g., the PubChem dataset, the QM9 molecule dataset, the Geometric Ensemble of Molecules (GEOM) Drugs dataset, and/or the like).

In the example of the molecule design computation model 115 shown in FIGS. 1A-1B, the molecule design computation model 115 may include the denoising model 117. Training the molecule design computation model 115 in this case may include training the denoising model 117 to approximate a noisy data distribution or, in some cases, a noisy latent distribution, either one of which exhibit smoother density transitions and is more efficient to sample from than the true data distribution. In some cases, the denoising model 117 may be an artificial neural network (ANN), in which case the training of the denoising model 117 may include adjusting one or more parameters (e.g., weights, biases, and/or the like) of the artificial neural network (ANN). Doing so may also determine the parameters of the function 175 such that the function 175 outputs a value indicative of the likelihood of a molecule exhibiting the one or more desired properties being at a particular location within the data distribution. For example, in some cases, the function 175 may be a score function whose output is a value (e.g., a score) indicative of the transitions between different density regions of the data distribution including, for example, transitions between higher density regions more likely to be occupied by molecules exhibiting the one or more desired properties and lower density regions of the data distribution less likely to be occupied by molecules exhibiting the one or more desired properties. In some cases, the denoising model 117 may be trained to recover the noisy three-dimensional representation 182 of the sample molecule or the embedding 186 in order to avoid overfitting the denoising model 117, for example, to the relatively few known molecules in the training dataset that are available to characterize the data distribution.

To further illustrate, let p(x) denote the true data distribution of the voxelized representation of molecules exhibiting the one or more desired properties and p(y) denote the corresponding noisy data distribution, which may exhibit a smoother energy landscape and is more efficient to sample from than the unknown data distribution p(x). In some cases, the true data distribution p(x) may be unknown, meaning that the denoising model 117 may be trained to approximate the true data distribution p(x) based on a training dataset of known molecules from the true data distribution. To avoid overfitting the denoising model 117 to the training dataset, the denoising model 117 may be trained to approximate the noisy data distribution p(y) instead. In some cases, the noisy data distribution p(y) may be obtained by convolving the true data distribution p(x) with a Gaussian kernel (e.g., an isotropic Gaussian kernel) with a known covariance σ²I_{d}. Doing so may be tantamount to generating a noisy voxelized molecule representation y by adding noise E to the voxelized molecule representation x from the true data distribution p(x) (e.g., y=x+ε, where x˜p(x), ε˜N(0,σ²I_{d})). Given the foregoing formulation, the noisy voxelized molecule representation Y may be sampled from the noisy data distribution p(y) expressed below:

$$p ( y ) = {\int }_{{R}^{d}} \frac{1}{{( 2 π {σ}^{2} )}^{\frac{d}{2}}} exp exp ( - \frac{{‖ y - x ‖}^{2}}{2 {σ}^{2}} ) p ( x ) dx$$

Transforming the true data distribution p(x) in this manner may smooth the densities of the true data distribution p(x) while still preserving some of the structural information present in the clean (or original) voxelized representation x absent any added noise ε. If the noise E added to the clean voxelized molecule representation x is Gaussian (e.g., isotropic Gaussian), the clean voxelized molecule representation x may be recovered directly from the corresponding noisy voxelized molecule representation y by applying the least-square estimator \hat{x}(y) shown as Equation (1) below. It should be appreciated that the least-square estimator \hat{x}(y) may act as a denoiser and recover the clean voxelized molecule representation x by removing the noise e present in the noisy voxelized molecule representation y.

$$\hat{x} ( y ) = y + {σ}^{2} {∇}_{y} log log p ( y ) , \qquad ( 1 )$$

wherein ∇_{y} log log p(y) corresponds to the score function g(y) of the noisy data distribution p(y). Equation (1) indicates that if the noisy data distribution p(y) is known up to a normalization constant (and thus the corresponding score function g(y)), then the clean voxelized molecule representation x can be estimated from its noisy counterpart y. Equivalently, the score function g(y) of the noisy data distribution p(y) can also be derived based on the least-square estimator \hat{x}(y) of the true data distribution p(x). As described in more details below, upon determining the score function g(y) or, in some cases, the corresponding score function, the molecule design computation model 115 may apply the denoising model 117 to sample from the noisy data distribution p(y) based on the score function g(y) (or the corresponding score function).

As described in more details below, once the denoising model 117 is trained, the molecule design computation model 115 may apply the denoising model 117 to perform a “walk-jump” generative process to generate output molecules that exhibit the one or more desired properties of the molecules in the true data distribution p(x). For example, in some cases, the denoising model 117 may sample from the noisy data distribution p(y) over multiple successive sampling iterations, each of which including the denoising model 117 selecting at least one sample from the noisy data distribution p(y) by at least denoising the noisy voxelized molecule representation y. In some cases, the sampling of the noisy data distribution p(y) may be guided by the score function g(y) such that the sample selected during one sample iteration originates from a different location in the noisy data distribution p(y) than the sample that is selected during another sample iteration. This traversal of the noisy data distribution p(y) is what is called the “walking” portion of the generative process.

In some cases, instead of sampling freely from the entire noisy data distribution p(y), the score function g(y) may restrict the sampling of molecules y to certain regions within the noisy data distribution p(y|c) based on a condition c (e.g., gradient of a classifier). Accordingly, in some cases, each successive sample may be selected from an incrementally higher density region of the noisy data distribution p(y), as indicated by the score output by the score function g(y). Again, as noted, traversing noisy data distribution p(y) while being guided by the score function g(y) may be considered “walking” the noisy data distribution p(y). The recovery of the clean voxelized molecule representation x from the corresponding noisy voxelized molecule representation y may constitute a “jump” from the noisy data distribution p(y) back to the true data distribution p(x). In some cases, the “jump” from the noisy data distribution p(y) back to the true data distribution p(x) may be accomplished by applying a denoiser, such as the denoising engine 117, to remove the noise e present in the noisy voxelized molecule representation y and recover the corresponding clean voxelized molecule representation x. For instance, in some cases, the clean voxelized molecule representation x may be recovered by the denoising engine 117 applying, to the noisy voxelized molecule representation y, the least-square estimator \hat{x}(y).

In some example embodiments, the training engine 120 may generate each corrupted sample molecule in the training dataset by adding noise to the embedding of the noisy three-dimensional representation of a sample molecule. For example, FIG. 1B shows that, in some cases, the corruption engine 121 may generate the corrupted embedding 188 by at least adding noise (e.g., Gaussian noise such as isotropic Gaussian noise and/or the like) to the embedding 186 of the noisy three-dimensional representation 182 (e.g., noisy voxelized representation) of the sample molecule (instead of directly to the noisy three-dimensional representation 182 of the sample molecule). FIG. 1B further shows that the embedding 186 may be generated by the encoder 111 down sampling (or compressing) the noisy three-dimensional representation 182 of the sample molecule. The down sampling (or compression) of the noisy three-dimensional representation 182 of the sample molecule may reduce the dimensionality of the noisy three-dimensional representation 182 of the sample molecule. For instance, where the noisy three-dimensional representation 182 of the sample molecule is a [32×32×32] voxel grid containing 32,000 features (or atomic density values), the down sampling (or compression) may yield a [4×4×4] voxel grid containing 64 features (or atomic density values). As such, the down sampling (or compression) of the noisy three-dimensional representation 182 of the sample molecule may increase the overall speed and efficiency of the generative process. In some cases, large quantities of candidate molecules, such as tens of thousands or even millions of candidate molecules, may be generated in a short period time to support low yield applications, such as computational drug design, where a large proportion of candidate molecules fail to be successfully synthesized in the laboratory. The down sampling (or compression) of the three-dimensional representation 182 of the sample molecule may also enable the generation of larger sized molecules (e.g., molecules containing upwards of 200 atoms), which may be overly cumbersome to operate upon if kept in their original three-dimensional representation without any down sampling (or compression).

At 204, the molecule design engine 110 may train the molecule design computation model 115 by at least applying the molecule design computation model 115 to recover the three-dimensional representation of each corrupted sample molecule in the training dataset from the corrupted three-dimensional representation of the sample molecule. In some example embodiments, the step 204 of training of the molecule design computation model 115 may include training, based at least on the training dataset, the denoising model 117 to approximate the noisy data distribution (or noisy latent distribution) of molecules exhibiting the one or more desired properties. For instance, in the example shown in FIG. 1A, the denoising model 117 may be trained to recover the noisy three-dimensional representation 182 (e.g., voxelized representation) of the sample molecule from the corrupted three-dimensional representation 184 of the sample molecule. In the example shown in FIG. 1B, the denoising model 117 may be trained to recover the embedding 186 of the noisy three-dimensional representation 182 of the sample molecule from the corrupted embedding 188. It should be appreciated that in either example, the denoising engine 117 may be trained on the noisy three-dimensional representation 182 of the sample molecule and not the clean three-dimensional representation of the sample molecule in order for the denoising engine 117 to approximate the noisy data distribution, which exhibits smoother density transitions than the true data distribution.

In the example shown in FIG. 1A, the training of the molecule design computation model 115 may include adjusting one or more parameters (e.g., weights, biases, and/or the like) of the denoising model 117 to reduce (or minimize) the difference (e.g., mean squared error (MSE)) between the noisy three-dimensional representation 182 of the sample molecule recovered by the denoising model 117 and the original noisy three-dimensional representation 182 of that sample molecule. Moreover, the training of the denoising model 117 may include determining the function 175, which is parameterized by the parameters (e.g., weights, biases, and/or the like) of the denoising model 117. For example, in some cases, the function 175 may be a score function that outputs a value (e.g., score) indicative of the local change in the density (or the gradient) of the data distribution. Accordingly, in some cases, the function 175 may output a first value (e.g., first score) indicative of a first local change in the density of the data distribution at a first location occupied by a first molecule and a second value (e.g., second score) indicative of a second local change in the density of the data distribution at a second location occupied by a second molecule. In some cases, the first value (e.g., first score) may indicate a more positive local change (e.g., an increase or a smaller decrease) in the density of the data distribution at the first location of the first molecule while the second value (e.g., second score) may indicate a less positive local change (e.g., a smaller increase or a decrease) in the density of the data distribution at the second location of the second molecule. In instances where the function 175 is a score function, the sampling of the data distribution may be guided by the values (e.g., scores) output by the function 175. As described in more details below, the sampling of the data distribution may be guided by the function 175 such that samples (or molecules) are selected from incrementally higher density regions of the data distribution, which are more likely to be occupied by molecules exhibiting the one or more desired properties.

In the example shown in FIG. 1B, the training of the denoising model 117 may include adjusting one or more parameters (e.g., weights, biases, and/or the like) of the denoising model 117 to reduce (or minimize) the difference (e.g., mean squared error (MSE)) between the original, uncorrupted embedding 186 of the noisy three-dimensional representation 182 of the sample molecule recovered by the denoising model 117 from the corrupted embedding 188 and the embedding 186. Doing so may also adjust the parameters of the function 175. Again, in instances where the function 175 is a score function, the parameters of the function 175 may be adjusted such that the function 175 outputs a higher value (e.g., higher score) for a first molecule occupying a first location in the data distribution exhibiting a more positive local change in the density of the data distribution (e.g., a positive gradient indicating a transition from a lower density to a higher density region of the data distribution) than a second molecule occupying a second location in the data distribution exhibiting a less positive local change in the density of the data distribution.

In some example embodiments, the molecule design engine 110 may train the molecule design computation model 115, including the denoising model 117, by at least performing a gradient based Markov Chain Monte Carlo (MCMC) sampling, such as Markov Chain Monte Carlo (MCMC) sampling with Langevin dynamics and/or the like, to approximate the function 175. In some cases, the function 175 may output values (e.g., scores) indicative of transitions between different density regions of the data distribution. For example, as a score function, the values (e.g., scores) output by the function 175 for each molecule may indicate the local change in density (or gradient) at the corresponding location in the data distribution. In the example shown in FIG. 1A, the gradient based Markov Chain Monte Carlo (MCMC) sampling to determine the function 175 may include adjusting the parameters (e.g., weights, biases, and/or the like) of the denoising model 117 and those of the function 175 over multiple iterations to increase (or maximize) the similarity (e.g., by reducing (or minimizing) the mean squared error (MSE)) between the noisy three dimensional representation 182 of the sample molecule recovered by the denoising model 117 and the original three-dimensional representation 182 of the sample molecule. For the example shown in FIG. 1B, the one or more parameters of the denoising model 117 and those of the function 175 may be adjusted over multiple iterations of gradient based Markov Chain Monte Carlo (MCMC) to increase (or maximize) the similarity (e.g., by reducing (or minimizing) the mean squared error (MSE)) between the embedding 186 of the noisy three-dimensional representation 182 of the sample molecule recovered by the denoising engine 117 from the corrupted embedding 188 and the original, uncorrupted embedding 186.

As noted, the denoising model 117 may be trained to recover the noisy three-dimensional representation 182 (e.g., noisy voxelized representation) of the sample molecule in order to avoid overfitting the denoising model 117 to the known molecules available for training the denoising model 117. In cases where relatively few known molecules characterizing a high-dimensional data distribution are available, training the denoising model 117 based on the known molecules directly may yield an overly jagged energy landscape in which drastic gradient changes are present between the regions populated by the known molecules. Sampling from the data distribution while being guided by steep gradients may prevent an adequate exploration of the data distribution at least because the steepness of the gradient may confine sampling to regions within the immediate vicinity of the known molecules. Contrastingly, training the denoising model 117 based on the noisy three-dimensional representation 182 (e.g., noisy voxelized representation) of the sample molecule may yield smoother density transitions, with the gradient of the function 175 being more gradual to enable a more efficient exploration of the data distribution when sampling therefrom.

At 206, the molecule design engine 110 may apply the trained molecule design computation model 115 to generate an output molecule by at least denoising a voxelized representation of an input molecule. In some example embodiments, the molecule design computation model 115 may use the denoising model 117 to generate the output molecule 162 by at least updating the three-dimensional representation (e.g., voxelized representation) of the input molecule 152 or, in some cases, the embedding 154 thereof, while being guided by the function 175. For example, molecule design computation model 115 of FIG. 1A may update the three-dimensional representation (e.g., voxelized representation) of the input molecule 152 directly, without any down sampling or compression. Alternatively, the molecule design computation model 115 of FIG. 1B may update the embedding 154 of the three-dimensional representation of the input molecule 152, which may be generated by the encoder 111 down sampling (or compressing) the three-dimensional representation (e.g., voxelized representation) of the input molecule 152. Doing so may reduce the dimensionality (or quantity of features) of the three-dimensional representation (e.g., voxelized representation) of the input molecule 152 such that the resultant embedding 154 may be more compact than the original (or uncompressed) three-dimensional representation of the input molecule 152. For instance, while the original three-dimensional representation (e.g., voxelized representation) of the input molecule 152 may include a [32×32×32] voxel grid containing 32,000 features (or atomic density values), the embedding 154 may include a [4×4×4] voxel grid containing 64 features (or atomic density values). It should be appreciated that the encoder 111 may be trained to down sample (or compress) the voxelized representation of the input molecule 152 such that the resulting embedding 154 conveys the same (or similar) information as the voxelized representation of the input molecule 152 in its original (or uncompressed) form. In some cases, the encoder 111 may be a part of an autoencoder (e.g., a variational autoencoder (VAE), such as a vector quantized variational autoencoder (VQ-VAE)), that also includes the decoder 119. In some cases, the encoder 111 may be trained to generate the embedding 154 such that the decoder 119 is able to recover the original voxelized representation of the input molecule 152 by at least decoding the embedding 154.

In some cases, the molecule design computation model 115 may denoise the input molecule 152 by at least updating the three-dimensional representation of the input molecule 152 or, alternatively, the embedding 154 of the three-dimensional representation of the input molecule 152. As noted, in some cases, the three-dimensional representation of the input molecule 152 may be a voxelized representation of the input molecule 152 in which the types of atoms and the positions of atoms present in the input molecule 152 are represented as continuous (e.g., Gaussian-like) atomic densities centered around the atoms. For example, in some cases, the voxelized representation of the input molecule 152 may include an [n×n×n] voxel grid containing an n³ quantity of voxels, each of which being associated with a value indicative of the atomic density at the corresponding location. In some cases, the atomic density associated with a single voxel may have a value between a range of values, such as [0,1], with an atomic density at the lower end of the range indicative of the voxel being farther away from any atoms in the input molecule 152 and an atomic density at the higher end of the range indicative of the voxel being closer to the center of an atom in the input molecule 152. Moreover, in some cases, the voxelized representation of the input molecule 152 may include multiple channels, with each channel corresponding to a different type of atom that may be present in the input molecule 152. Accordingly, in some cases, the voxelized representation of the input molecule 152 may represent the types of atoms and the positions of the atoms present in the input molecule 152 as continuous (e.g., Gaussian-like) atomic densities across one or more channels.

In some example embodiments, the denoising of the input molecule 152 may include updating the three-dimensional representation of the input molecule 152 or, alternatively, the embedding 154 of the input molecule 152. In instances where the molecule design computation model 115 operates on the three-dimensional representation of the input molecule 152 or in instances where the embedding 154 is generated without any down sampling (or compressing) of the three-dimensional representation of the input molecule 152, the denoising may include updating the atomic density of one or more voxels in at least one channel of the noisy voxelized representation of the input molecule 152. Doing so may be tantamount to adding, removing, and/or repositioning one or more atoms of different atomic types in the input molecule 152. For instance, increasing (or decreasing) the atomic density of one or more voxels in one channel of the noisy voxelized representation of the input molecule 152 may be tantamount to adding (or removing) a corresponding type of atom to the input molecule 152. Alternatively and/or additionally, decreasing the atomic density of a first voxel while increasing the atomic density of a second voxel may be tantamount to repositioning an atom from a first location of the first voxel to a second location of the second voxel.

Alternatively, in instances where the molecule design computation model 115 operates on the embedding 154 of the three-dimensional representation (e.g., voxelized representation) of the input molecule 152, the denoising may include updating the values of the voxels present in the embedding 154. As noted, the embedding 154 may be generated by the encoder 111 condensing at least some of the features (e.g., atomic density values) present in the voxelized representation of the input molecule 152. The embedding 154 may include a fewer quantity of features than the original voxelized representation of the input molecule 152 but still convey the same (or similar) information as the original three-dimensional representation of the input molecule 152. Accordingly, the denoising of the embedding 154 may include updating one or more values present in the embedding 154, at least some of which may be representative of multiple features (or atomic density values) from the original voxelized representation of the input molecule 152.

Updating the three-dimensional representation of the input molecule 152 or the embedding 154 thereof in the foregoing manner, may comprise selecting samples (or updated molecules) from the noisy data distribution (or noisy latent distribution) of molecules exhibiting the one or more desired properties. In the case of gradient-based Markov Chain Monte Carlo (MCMC) sampling, the updating may be guided by the output of the function 175 (e.g., the score output by the function 175) such that the samples (or updated molecules) selected during each successive sampling iteration originate from incrementally higher density regions of the noisy data distribution, which are more likely to be populated by molecules exhibiting the one or more desired properties.

To further illustrate, in some cases, the three-dimensional representation of the input molecule 152 or the embedding 154 thereof may undergo a first update and a second update. Doing so may be tantamount to selecting, from the noisy data distribution, a first sample (or first updated molecule) and a second sample (or second updated molecule). In some cases, upon selecting the first sample (or first updated molecule) and the second sample (or second updated molecule) from the noisy data distribution (or noisy latent distribution), the molecule design computation model 115 may apply the function 175 to determine a value (e.g., a score and/or the like) indicative of the likelihood of each sample (or updated molecule) within the noisy data distribution (or noisy latent distribution). In instances where the function 175 is an score function, for example, a higher value (e.g., lower score) may indicate that a sample (or updated molecule) is selected from region of the noisy data distribution exhibiting a greater positive local change (e.g., an increase or a smaller decrease) in density or, analogously, that the sample (or updated molecule) has a higher likelihood of being within the noisy data distribution. As such, in some cases, upon selecting the first sample (or first updated molecule) and the second sample (or second updated molecule), the molecule design computation model 115 may apply the denoising model 117 to continue updating the three-dimensional representation of the input molecule 152 or the embedding 154 thereof in order to select additional samples (or further updated molecules) from incrementally higher density regions of the noisy data distribution until, for example, a sample (or updated molecule) exhibiting a threshold likelihood of being within the noisy data distribution (or noisy latent distribution) is selected. For instance, in some cases, the denoising model 117 may be applied to further modify the three-dimensional representation of the input molecule 152 or the embedding 154 having the first update (or the first updated molecule) instead of the second update (or the second updated molecule) if the three-dimensional representation of the input molecule 152 or the embedding 154 having the first update (or the first updated molecule) is selected from a higher density region of the data distribution. Doing so may be analogous to traversing the noisy data distribution (or noisy latent distribution) to sample from incrementally higher density regions of the noisy data distribution. In instances where the denoising model 117 is modifying the embedding 154 of the three-dimensional representation of the input molecule 152, the denoising model 117 may be operating in a noisy latent space in which the distance between two or more embeddings therein may be reflective of the similarities (or dissimilarities) in the types and positions of atoms in different molecules. The sharp transitions in densities present of the true data distribution of molecules exhibiting the one or more desired properties may be smoothed by the addition of noise. Since the denoising model 117 is trained to approximate the data distribution of molecules exhibiting certain desired properties (e.g., drug-like properties), the updates made to the embedding 154 when denoising the input molecule 152 may be consistent with the types and positions of the atoms found in molecules that exhibit the one or more desired properties. As such, the same desired properties may also present in the output molecule 162 generated by the molecule design computation model 115 applying the denoising model 117 to denoise the input molecule 152.

FIG. 3A depicts a flowchart illustrating an example of a process 300 for training the molecule design computation model 115, in accordance with some example embodiments. Referring to FIGS. 1-2 and 3A, the process 300 may implement operation 204 of the process 200 shown in FIG. 2. In some cases, the process 300 may be performed by the molecule design engine 110 to train the molecule design computation model 115 including, for example, the denoising model 117, to approximate a noisy data distribution of the noisy three-dimensional representations (e.g., noisy voxelized representations) of molecules exhibiting one or more desired properties. As described in more details below, in some cases, the molecule design computation model 115, including the denoising model 117, may be trained to approximate the noisy data distribution instead of the true data distribution to avoid overfitting the molecule design computation model 115 to the known molecules available to train the molecule design computation model 115. Moreover, in some cases, the molecule design computation model 115, including the denoising model 117, may be trained through gradient based Markov Chain Monte Carlo (MCMC) sampling including, for example, Markov Chain Monte Carlo (MCMC) sampling with Langevin dynamics and/or the like).

At 302, the molecule design engine 110 may apply a molecule design computation model having a first adjustment to denoise a corrupted sample molecule and generate a first updated molecule. In some example embodiments, step 302 may include the molecule design engine 110 training the molecule design computation model 115 including, for example, the denoising model 117 to approximate the data distribution of the three-dimensional representations (e.g., voxelized representations) of molecules exhibiting one or more desired properties such that candidate molecules exhibiting the same desired properties can be generated by sampling therefrom. In some cases, the molecule design computation model 115 may be trained to approximate the aforementioned data distribution based on a training dataset of corrupted sample molecules, each of which being generated based on the noisy three-dimensional representation (e.g., voxelized representation) of a sample molecule (e.g., known molecule) from the data distribution. An example of this is shown in FIG. 1A in which the molecule design computation model 115 (e.g., the denoising model 117) is trained to recover, from the corrupted three-dimensional representation 184 of the sample molecule generated by the corruption engine 121, the noisy three-dimensional representation 182 of the sample molecule. In some cases, instead of being trained to directly recover the noisy three-dimensional representations (e.g., voxelized representations) of the sample molecules, the molecule design computation model 115 may be trained based on the corrupted embeddings of those three-dimensional representations (e.g., voxelized representations). This is shown in FIG. 1B where the molecule design computation model 115 (e.g., the denoising model 117) is trained to recover, from the corrupted embedding 188 generated by the corruption engine 121, the embedding 186 of the noisy three-dimensional representation 182 of the sample molecule.

In some example embodiments, the training of the molecule design computation model 115 may include applying the denoising model 117 to denoise the corrupted three-dimensional representation (e.g., voxelized representation) of each sample molecule or, alternatively, the corrupted embedding thereof. FIG. 1A shows one example in which the training of the molecule design computation model 115 includes adjusting the parameters (e.g., weights, biases and/or the like) of the denoising model 117 to decrease, for example, incrementally over multiple iterations, the difference (e.g., mean squared error (MSE)) between the noisy three-dimensional representations (e.g., voxelized representations) of the sample molecules and those recovered by the denoising model 117 denoising the corrupted three-dimensional representations of the sample molecules. Alternatively, in the example shown in FIG. 1B, the molecule design computation model 115 may be trained by adjusting the parameters (e.g., weights, biases, and/or the like) of the denoising model 117 to decrease, incrementally over multiple iterations, the difference (e.g., mean squared error) between the embeddings of the noisy three-dimensional representations of sample molecules and the embedding that the denoising model 117 recovers from the corresponding corrupted embeddings. In some cases, the parameters (e.g., weights, biases, and/or the like) of denoising model 117 may undergo different adjustments before further adjustments are made to the adjustment that yields a lower difference (e.g., mean squared error (MSE)). For example, in some cases, a first adjustment may be made to the parameters (e.g., weights, biases, and/or the like) of the denoising model 117 before the denoising model 117 having the first adjustment is applied to denoise the corrupted three-dimensional representation of a sample molecule or the corrupted embedding of the three-dimensional representation of the sample molecule and generate at least a first updated molecule. In some cases, the first updated molecule may be an updated three-dimensional representation (e.g., voxelized representation) of a first molecule or, alternatively, an updated embedding of the three-dimensional representation (e.g., voxelized representation) of the first molecule.

In some cases, the corrupted three-dimensional representation or the corrupted embedding of the three-dimensional representation of the sample molecule may be denoised by updating one or more atomic density values representative of the types and positions of the atoms present in the sample molecule. In instances where the corrupted embedding is generated by down sampling (or compressing) the three-dimensional representation of the sample molecule, at least some of the values being updated may condense multiple features (or atomic density values) from the original three-dimensional representation (e.g., voxelized representation) of the sample molecule. As described in more details below, the denoising model 117 having a second adjustment (instead of the first adjustment) may be applied to denoise the corrupted three-dimensional representation (e.g., corrupted voxelized representation) of the sample molecule or the corrupted embedding of the three-dimensional representation of the sample molecule and generate at least a second updated molecule. Further adjustments may be made to the denoising model 117 having either the first adjustment or the second adjustment. Doing so may train the denoising model 117 to approximate the noisy data distribution or, in some cases, a noisy latent distribution, which exhibits smoother density transitions to support more efficient sampling due to the absence of steep gradient changes that confine sampling to regions within the immediate vicinity of the sample molecules forming the basis of the training dataset.

In some example embodiments, the training of the denoising model 117 may further include determining the function 175. As noted, in some cases, the function 175 may be a score function parameterized by the parameters (e.g., weights, biases, and/or the like) of the denoising model 117. Accordingly, in some cases, training the molecule design computation model 115, which includes adjusting the parameters of the denoising model 117, may also include adjusting the parameters of the function 175. For example, in some cases, the function 175 may be determined by performing gradient-based Markov Chain Monte Carlo (MCMC) sampling (e.g., Langevin Markov Chain Monte Carlo (MCMC) sampling and/or the like) to approximate the gradient of the noisy data distribution (or noisy latent distribution). Doing so may include adjusting, over one or more iterations, the parameters of the function 175 such that the function 175 outputs a value (e.g., score) indicative of the local density change in the noisy data distribution (or noisy latent distribution). In instances where the function 175 is a score function, the parameters of the function 175 may be adjusted such that the function 175 assigns a higher value (e.g., higher score) to a sample, such as a three-dimensional representation of a molecule or an embedding thereof, from a location exhibiting a more positive local change (e.g., an increase or a smaller decrease) in density than one from a location exhibiting a less positive local change (e.g., a decrease or a smaller increase) in density. Accordingly, once the denoising model 117 is trained, the function 175 may output values (e.g., scores and/or the like) that differentiate between samples (e.g., three-dimensional representations, embeddings of three-dimensional representations, and/or the like) from higher density regions of the noisy data distribution (or noisy latent distribution) and those sampled from lower density regions of the noisy data distribution.

At 304, the molecule design engine 110 may apply the molecule design computation model having a second adjustment to denoise the corrupted sample molecule and generate a second updated molecule. In some example embodiments of step 304, upon applying the denoising model 117 having the first adjustment to generate at least the first updated molecule, the denoising model 117 having a second adjustment may be applied to generate at least a second updated molecule such as, for example, an updated three-dimensional representation (e.g., updated voxelized representation of a second molecule or an updated embedding of the three-dimensional representation of the second molecule. It should be appreciated that the first adjustment and the second adjustment may include different changes to the parameters (e.g., weights, biases, and/or the like) of the denoising model 117. As such, applying the denoising model 117 having the second adjustment to denoise the corrupted three-dimensional representation 184 of the sample molecule or the corrupted embedding 186 of the noisy three-dimensional representation 182 of the sample molecule may yield different updated molecules than applying the denoising model 117 having the first adjustment to denoise the corrupted three-dimensional representation 182 of the sample molecule or the corrupted embedding 186 of the noisy three-dimensional representation 182 of the same sample molecule. As described in more details below, the training of the denoising model 117 may include further adjusting the denoising model 117 having either the first adjustment or the second adjustment depending on the difference (e.g., mean squared error (MSE)) present in the noisy three-dimensional representation 182 of the sample molecule (FIG. 1A) or the embedding 184 of the noisy three-dimensional representation 182 of the sample molecule (FIG. 1B) recovered by the denoising model 117.

At 306, the molecule design engine 110 may determine that the first updated molecule is more similar to the sample molecule than the second updated molecule. In some example embodiments, step 306 may include the molecule design engine 110 selecting, for further adjustments during a subsequent iteration, the denoising model 117 having the first adjustment instead of the denoising model 117 having the second adjustment if the first updated molecule generated by the denoising model 117 having the first adjustment is more similar (e.g., exhibits a lower mean squared error (MSE)) to the noisy three-dimensional representation of the sample molecule (or the embedding thereof) than the second updated molecule generated by the denoising model 117 having the second adjustment. For example, in FIG. 1A, the first updated molecule may be an updated three-dimensional representation of a first molecule with a smaller difference (e.g., lower mean squared error (MSE)) relative to the noisy three-dimensional representation 182 of the sample molecule than the second updated molecule. In FIG. 1B, the first updated molecule may be an updated embedding of the three-dimensional representation of the first molecule that has a smaller difference (e.g., lower mean squared error (MSE)) relative to the embedding 186 of the noisy three-dimensional representation 182 of the sample molecule than the second updated molecule.

That the first updated molecule is more similar to the noisy three-dimensional representation 182 of the sample molecule (or the embedding 186 thereof) than the second updated molecule may indicate that the denoising model 117 having the first adjustment is better at recovering the noisy three-dimensional representation 182 of the sample molecule (or the embedding 186 thereof) than the denoising model 117 having the second adjustment. The denoising model 117 having the first adjustment may therefore better approximate the noisy data distribution (or noisy latent distribution) of molecules exhibiting the one or more desired properties than the denoising model 117 having the second adjustment. Accordingly, in some cases, the molecule design engine 110 may select the denoising model 117 having the first adjustment instead of the denoising model 117 having the second adjustment to undergo one or more additional iterations of adjustments.

At 308, the molecule design engine 110 may further adjust, until one or more criteria are met, the molecule design computation model having the first adjustment instead of the second adjustment. In some example embodiments, the molecule design engine 110 may further adjust the denoising model 117 having the first adjustment instead of the denoising model 117 having the second adjustment in instances where the first updated molecule generated by the denoising model 117 having the first adjustment is more similar (e.g. lower mean squared error (MSE)) to the noisy three-dimensional representation 182 of the sample molecule (or the embedding 186 thereof) than the second updated molecule generated by the denoising model 117 having the second adjustment. For example, during a subsequent iteration of adjustments, the molecule design engine 110 may further adjust to the parameters (e.g., weights, biases, and/or the like) the denoising model 117 having the first adjustments before applying the further adjusted denoising model 117 to generate one or more additional updated molecules. In some cases, the denoising model 117 may be further adjusted in order to further increase the similarity (or lower the mean squared error (MSE)) between the updated molecules generated by the denoising model 117 and the noisy three-dimensional representations of the sample molecules (or the embeddings thereof) in the training dataset. In some cases, the molecule design engine 110 may continue to adjust the denoising model 117 until one or more criteria are satisfied. For instance, in some cases, the molecule design engine 110 may continue to adjust the parameters (e.g., weights, biases, and/or the like) of denoising model 117 until the molecule design engine 110 has performed a threshold quantity of iterations of adjustments. Alternatively and/or additionally, the molecule design engine 110 may continue to adjust the parameters (e.g., weights, biases, and/or the like) of denoising model 117 until the similarity (e.g., mean squared error (MSE)) between the updated molecules generated by the denoising model 117 and the noisy three-dimensional representations of the sample molecules (or the embeddings thereof) in the training dataset satisfies one or more thresholds. In some cases, the molecule design engine 110 may continue to adjust the parameters (e.g., weights, biases, and/or the like) of the denoising model 117 until the updated molecules generated by the denoising model 117 exhibits a threshold likelihood of being in the data distribution of the molecules exhibiting the one or more desired properties training dataset.

As described in more details below, once the one or more criteria are met, the trained denoising model 117 may be applied to generate the three-dimensional representation (e.g., voxelized representation) of the output molecule 162, by at least denoising the three-dimensional representation (e.g., voxelized representation) of the input molecule 152. As shown in FIGS. 1A-B, the trained denoising model 117 may generate the three-dimensional representation (e.g., voxelized representation) of the output molecule 162 by at least sampling, based on the function 175, from a noisy data distribution populated by noisy three dimensional representations (e.g., voxelized representations) of molecules exhibiting one or more desired properties (e.g., drug-like properties) or, alternatively, a noisy latent distribution populated by embeddings of the noisy three-dimensional representations (e.g., voxelized representations) of the molecules. The sampling may include one or more iterations of gradient-based Markov Chain Monte Carlo (MCMC) sampling (e.g., Langevin Markov Chain Monte Carlo and/or the like), which may be guided by the function 175 such that each sampling iteration include selecting one or more samples (or molecules) from incrementally higher density regions of the noisy data distribution (or noisy latent distribution).

FIG. 3B depicts a flowchart illustrating an example of a process 325 for applying a molecule design computation model to generate three-dimensional molecules in voxelized space, in accordance with some example embodiments. Referring to FIGS. 1A, 1B, 2 and 3B, the process 325 may implement operation 206 of the process 200 shown in FIG. 2. In some cases, the process 325 may be performed by the molecule design engine 110. For example, in some cases, the molecule design engine 110 may apply the molecule design computation model 115 (e.g., the denoising model 117) to generate a three-dimensional representation (e.g., voxelized representation) of the output molecule by at least denoising the three-dimensional representation (e.g., voxelized representation) of the input molecule. In some cases, the input molecule may be a random molecule (e.g., a molecule having a random selection of atomic types and/or positions) or a known molecule having one or more desired properties. Accordingly, the three-dimensional representation (e.g., voxelized representation) of the input molecule may include noise that require removal by the molecule design computation model 115 in order for the three-dimensional representation (e.g., voxelized representation) of the resultant output molecule to be consistent with the molecules exhibiting one or more desired properties (e.g., drug-like properties). The molecule design computation model 115 may denoise the three-dimensional representation (e.g., voxelized representation) of the input molecule by at least sampling from a noisy data distribution (or a noisy latent distribution) that is more efficient to sample from because the smoother density transitions present therein permits an adequate exploration of the data distribution. As described in more details below, once the molecule design computation model 115 generates the three-dimensional representation (e.g., voxelized representation) of the output molecule, the molecule design engine 110 may further generate one or more other representations of that output molecule including, for example, a one-dimensional representation of the output molecule, a two-dimensional representation of the output molecule, and/or the like. That the output molecule is generated by operating on the three-dimensional representation (e.g., voxelized representation) of the input molecule, which captures the conformation (or three-dimensional structure) of the input molecule, means that the conformation (or three-dimensional) structure of the output molecule is more likely to be consistent with one or more desired properties (e.g., drug-like properties such as affinity, specificity, biological activity, developability, and/or the like).

At 332, the molecule design engine 110 may update a three-dimensional representation of an input molecule to generate an updated three-dimensional representation. In some example embodiments, updating the three-dimensional representation may include the molecule design engine 110 applying the molecule design computation model 115 to generate the three-dimensional representation (e.g., voxelized representation) of the output molecule 162 by at least denoising the three-dimensional representation (e.g., voxelized representation) of the input molecule 152. An example of this process is shown in FIG. 1A in which the denoising engine 117 denoises the three-dimensional representation of the input molecule 152 to generate the updated three-dimensional representation 160. In some cases, the input molecule 152 may be a noise molecule (e.g., a molecule with a random selection of atomic types and/or positions) or a known molecule having one or more undesirable properties. This means that the three-dimensional representation of the input molecule 152 may include at least some noise that renders it inconsistent with that of a molecule exhibiting one or more desired properties (e.g., drug-like properties). As such, in some cases, the denoising engine 117 may be trained to update the three-dimensional representation of the input molecule 152 such that the resulting updated three-dimensional representation 160 is consistent with that of a molecule exhibiting the one or more desired properties.

In some example embodiments, the molecule design computation model 115 may apply the denoising model 117 to update the three-dimensional representation of the input molecule 152 based on the function 175. In some cases, the function 175 may be a score function that outputs, for each sample (or molecule) selected from the noisy data distribution, a value (e.g., a score and/or the like) indicative of the likelihood of the sample (or molecule) being in the noisy data distribution. For example, in some cases, the value output by the function 175 for a particular sample (or molecule) may indicate the local change in density at the location from which the sample (or molecule) is selected. The denoising model 117 may update, based at least on the values output by the function 175, the three-dimensional representation of the input molecule 152 over multiple successive sampling iterations. During each sampling iteration, the denoising model 117 may be applied to further update the three-dimensional representation of the input molecule 152 such that the resulting updated three-dimensional representation 160 is selected from a higher density region of the noisy data distribution than what is selected during one or more previous sampling iterations.

In some example embodiments, the molecule design computation model 115 may perform a gradient based Markov Chain Monte Carlo (MCMC) sampling (e.g., Langevin Markov Chain Monte Carlo (MCMC) sampling) of the noisy data distribution in which the three-dimensional representation (e.g., voxelized representation) of the input molecule 152 is updated over multiple successive sampling iterations. In some cases, each iteration may include the molecule design computation model 115 further updating the three-dimensional representation of the input molecule 152 to sample from an incrementally higher density region of the noisy data distribution. Moreover, in some cases, the updates made to the three-dimensional representation of the input molecule 152 may be cumulative over the multiple successive iterations. For example, in some cases, the three-dimensional representation of the input molecule 152 may undergo a first update and a second update. The molecule design computation model 115 may apply the function 175 to determine a first value (e.g., first score and/or the like) for the three-dimensional representation of the input molecule 152 having the first update and a second value (e.g., second score and/or the like) for the three-dimensional representation of the input molecule 152 having the second update. During a subsequent iteration of gradient-based Markov Chain Monte Carlo (MCMC) sampling, the denoising model 117 may be applied to further update the three-dimensional representation of the input molecule 152 having the first update if the first value and the second value indicate that the three-dimensional representation of the input molecule 152 having the first update is sampled from a higher density region of the noisy data distribution and exhibits a higher likelihood of being within the noisy data distribution than the three-dimensional representation of the input molecule 152 having the second update.

In some cases, one or more additional iterations of the gradient-based Markov Chain Monte Carlo (MCMC) sampling may be performed, with the molecule design computation model 115 applying the denoising model 117 to further modify the three-dimensional representation of the input molecule 152 until one or more criteria are met. For example, in some cases, the molecule design computation model 115 may perform one or more additional iterations of gradient based Markov Chain Monte Carlo (MCMC) sampling until a threshold quantity of sampling iterations are performed. Alternatively and/or additionally, the molecule design computation model 115 may perform one or more additional iterations of gradient based Markov Chain Monte Carlo (MCMC) sampling until the function 175 outputs, for the updated three-dimensional representation 160, a value (e.g., score and/or the like) satisfying one or more thresholds. That the value (e.g., score and/or the like) associated with the updated three-dimensional representation 160 satisfies the one or more thresholds may indicate that the updated three-dimensional representation 160 is selected from a region of the noisy data distribution having a sufficiently high density and that the likelihood of the updated three-dimensional representation 160 being within the noisy data distribution satisfies one or more thresholds. In some cases, the one or more criteria may also include having generated a threshold quantity of output molecules exhibiting the one or more desired properties (e.g., at least one output molecule exhibiting a threshold level of one or more drug-like properties such as affinity, specificity, biological activity, developability, and/or the like).

At 336, the molecule design engine 110 may denoise the updated three-dimensional representation to generate a three-dimensional representation of an output molecule. In some example embodiments of step 336, the molecule design computation model 115 may denoise the three-dimensional representation of the input molecule 152 by sampling from a noisy data distribution occupied by noisy three-dimensional representations of molecules exhibiting one or more desired properties. As noted, the molecule design computation model 115, including the denoising model 117, may be trained to approximate the noisy data distribution (instead of the true data distribution) by at least being trained to denoise the corrupted three-dimensional representation 184 of the sample molecule to recover the noisy three-dimensional representation 182 of the sample molecule and not the clean three-dimensional representation of the sample molecule. Moreover, this noisy data distribution may exhibit smoother density transitions and is therefore more efficient to sample from. That the updated three-dimensional representation 160 is sampled from the noisy data distribution means that the updated three-dimensional representation 160 may undergo additional denoising. For example, FIG. 1A shows that the molecule design engine 110 may apply the recovery model 118 in order to denoise the updated three-dimensional representation 160 and generate the three-dimensional representation (e.g., voxelized representation) of the output molecule 162 therefrom. In some cases, the recovery model 118 may be trained to denoise the updated three-dimensional representation 160 in order to map the updated three-dimensional representation 160 from the noisy data distribution back to the true data distribution of the molecules exhibiting the one or more desired properties (e.g., drug-like properties). It should be appreciated that this denoising is different from the denoising the denoising model 117 is trained to perform, which includes updating the three-dimensional representation of the input molecule 152 to sample from a higher density region of the noisy data distribution more likely to be occupied by molecules exhibiting the one or more desired properties.

At 338, the molecule design engine 110 may generate, based at least on the three-dimensional representation of the output molecule, one or more other representations of the output molecule. In some example embodiments, the three-dimensional representation (e.g., voxelized representation) of the output molecule 162, which is generated by the recovery model 118 denoising the updated three-dimensional representation 160 sampled from the noisy data distribution by the molecule computation model 115, may be further transformed into one or more other representations of the output molecule 162. For example, in some cases, the molecule design engine 110 may recover, based at least on the three-dimensional representation (e.g., voxelized representation) of the output molecule 162, the positions (e.g., coordinates) of the atoms present in the output molecule 162 and one or more bonds therebetween. In doing so, the molecule design engine 110 may determine another representation of the output molecule 162 including, for example, a one-dimensional representation of the output molecule 162 (e.g., a simplified molecular-input line-entry system (SMILES) string), a two-dimensional representation of the output molecule 162 (e.g., a molecular graph), and/or the like. In some cases, the molecule design engine 110 may recover the positions of the atoms present in the output molecule 162 by applying a peak detection technique, which determines the positions (e.g., coordinates) of the atoms based on one or more peaks in the atomic densities included in the three-dimensional representation (e.g., voxelized representation) of the output molecule 162 before determining, based on the positions of the atoms, one or more interconnecting bonds. Alternatively, the molecule design engine 110 may apply a machine learning model trained to translate the voxelized representation of the output molecule 162 into one or more other representations.

FIG. 3C depicts a flowchart illustrating an example of a process 350 for applying a molecule design computation model to generate three-dimensional molecules in voxelized space, in accordance with some example embodiments. Referring to FIGS. 1-2 and 3C, the process 350 may implement operation 206 of the process 200 shown in FIG. 2. In some cases, the process 350 may be performed by the molecule design engine 110. For example, in some cases, the molecule design engine 110 may apply the molecule design computation model 115 (e.g., the denoising model 117) to generate a three-dimensional representation of an output molecule, such as a voxelized representation of the output molecule, by at least denoising the three-dimensional representation (e.g., voxelized representation) of an input molecule. In some cases, the three-dimensional representation of the input molecule may be denoised by at least updating an embedding of the three-dimensional representation (e.g., voxelized representation) of the input molecule and not the three-dimensional representation of the input molecule directly at least because the embedding may be more compact and more computationally efficient to operate upon. In some cases, the embedding of the three-dimensional representation of the input molecule may be generated by down sampling (or compressing) the three-dimensional representation of the input molecule although it is also possible for the embedding to be generated without any down sampling (or compression) of the three-dimensional representation of the input molecule. In the former case, the embedding of the three-dimensional representation of the input molecule may occupy a latent voxelized space whereas in the latter, the embedding of the three-dimensional representation of the input molecule may remain in the same discrete voxelized space as the original three-dimensional representation of the input molecule. It should be appreciated that the latent voxelized space may have a lower dimensionality than the discrete voxelized space such that operating on the embedding of the three-dimensional representation of the input molecule may increase the speed and computational efficiency of the generative process while achieving comparable or better generative performance.

It should be appreciated that the molecule design computation model 115 may denoise the embedding of the three-dimensional representation of the input molecule by sampling from a noisy latent distribution. That is, as noted, the molecule design computation model 115 may be trained to approximate the noisy latent distribution and not the true data distribution to at least avoid the steep density transitions that are present in the true data distribution. In other words, the updated embeddings that are generated by the molecule design computation model 115 updating the embedding of the three-dimensional representation of the input molecule may still occupy a noisy latent distribution. This noisy latent distribution may be more efficient to sample from because the smoother density transitions of the noisy latent distribution support an adequate exploration of the data distribution. As described in more details below, the updated embeddings may undergo decoding and further denoising in order to “jump” back to the true data distribution. Furthermore, in some cases, the molecule design engine 110 may generate, based on the three-dimensional representation of the output molecule resulting from the decoding and denoising of an updated embedding, one or more other representations of that output molecule including, for example, a one-dimensional representation of the output molecule, a two-dimensional representation of the output molecule, and/or the like. That the output molecule is generated by operating on the three-dimensional representation (e.g., voxelized representation) of the input molecule, which captures the conformation (or three-dimensional structure) of the input molecule, means that the conformation (or three-dimensional) structure of the output molecule is more likely to be consistent with one or more desired properties (e.g., drug-like properties such as affinity, specificity, biological activity, developability, and/or the like).

At 352, the molecule design engine 110 may encode a three-dimensional representation of an input molecule to generate an embedding of the input molecule. In some example embodiments, the encoder 111 may encode the three-dimensional representation (e.g., voxelized representation) of the input molecule 152 to generate the embedding 154 of the input molecule 152. One example of this is shown in FIG. 1B. In the case of “seeded generation,” the input molecule 152 may be a known molecule (e.g., a molecule from a validation set derived from the PubChem dataset, the QM9 molecule dataset, the Geometric Ensemble of Molecules (GEOM) Drugs dataset, and/or the like). In some cases, the known molecule may exhibit one or more undesirable properties. Where a known molecule is used as the input molecule 152, the generative process may be initialized with a voxel grid having a distribution of atomic densities corresponding to the types and positions of atoms expected to be found in the known molecule. Alternatively, the molecule design computation model 115 may perform de novo generation, in which case the input molecule 152 may be a noise molecule whose atomic types and positions correspond to pure noise (e.g., uniform noise and/or the like). In instances where a noise molecule is used as the input molecule 152, the generative process may be initialized to the entire voxel grid, without any expectation for the atomic types and/or positions. In either case, the types and/or the positions of the atoms in the input molecule 152 may be inconsistent with that of molecules exhibiting the one or more desired properties (e.g., drug-like properties). Hence, the molecule design computation model 115 may be applied to update the three-dimensional representation of the input molecule 152 by at least updating the embedding 154 and generate the updated embedding 156 such that the corresponding three-dimensional representation of the output molecule 162 may be more consistent with those of the molecules exhibiting the one or more desired properties.

In some example embodiments, the encoder 111 may encode the three-dimensional representation (e.g., voxelized representation) of the input molecule 152 by at least down sampling or compressing the three-dimensional representation of the input molecule 152. Doing so may include condensing at least some of the features present in the three-dimensional representation of the input molecule 152, which reduces the dimensionality (or quantity of features) present in the three-dimensional representation of the input molecule 152. For example, in cases where the three-dimensional representation of the input molecule 152 includes a [32×32×32] voxel grid containing 32,000 features (or atomic density values), the encoder 111 may condense at least some of those 32,000 features (or atomic density values) to generate, as the embedding 154 of the input molecule 152, a [4×4×4] voxel grid containing 64 features.

In some example embodiments, the encoder 111 may generate the embedding 154 of the input molecule 152 with or without down sampling or compressing the three-dimensional representation (e.g., voxelized representation) of the input molecule 152. In some cases, the encoder 111 may implement an identity function, meaning that the embedding 154 may include the same quantity of features (e.g., atomic density values) present in the three-dimensional representation of the input molecule 152. Alternatively, in instances where the embedding 154 is generated by down sampling the voxelized representation of the input molecule 152, doing so may project the voxelized representation of the input molecule 152 from a higher dimensional discrete voxelized space to a lower dimensional latent space. Sampling from the lower dimensional latent space may impose less computational burden than sampling directly from the higher dimensional discrete voxelized space. For example, in cases where sampling from the discrete voxelized space is a resource intensive task, such as when the input molecule 152 is large in size (e.g., containing between 80 to 200 atoms) or when a large quantity candidate molecules are being generated therefrom, the molecule design engine 110 may sample from the latent voxelized space by applying the molecule design computation model 115 to operate on the embedding 154 of the three-dimensional representation of the input molecule 152. It should be appreciated that sampling from the latent voxelized space may impose moderate computational overhead even in cases where the input molecule 152 is large in size (e.g., containing upwards of 200 atoms) or when a large quantity of candidate molecules are being generated.

In some example embodiments, the encoder 111 may be a part of an autoencoder (e.g., a variational autoencoder (VAE) such as a vector quantized variational autoencoder (VQ-VAE)) along with the decoder 119. In some cases, the encoder 111 may be trained to encode the voxelized representation of the input molecule 152 such that the decoder 119 is able to recover the three-dimensional representation (e.g., voxelized representation) of the input molecule 152 from the resulting embedding 154. To further illustrate, let x denote voxelized representations of molecules, fₑ denote the encoder 111, f_{d} denote the decoder 119, and zₑ the embedding 154. The voxelized molecule representations x, such as that of the input molecule 152, for example, may be encoded with the encoder fₑ(x) to generate the continuous latent embeddings zₑ(x) in accordance with Equation (2) below.

$${f}_{e} ( {θ}_{e} ) : x \to {z}_{e} ( x ) \qquad ( 2 )$$

According to Equation (3), each of the continuous latent embeddings zₑ(x) may be quantized to a discrete latent embedding z by matching with one of k vectors in a learned shared codebook of embeddings e by a nearest neighbor lookup.

$${z}_{q} ( x ) = {e}_{k} , where k = arg {min}_{j} {‖ {z}_{e} ( x ) - {e}_{j} ‖}_{2} \qquad ( 3 )$$

The quantized latent embeddings z_{q}(x) may be passed through the decoder f_{d} to reconstruct the original voxelized molecule representations x in accordance with Equation (4) below.

$${f}_{d} ( {θ}_{d} ) : {z}_{q} ( x ) \to \hat{x} \qquad ( 4 )$$

The latent embedding space may be denoted as e∈R^{K×d}, where K is the quantity of discrete latent vectors in the codebook that is learned and d is the dimensionality of each latent embedding vector in the codebook. It should be appreciated that K and d are hyperparameters whose selection may be made experimentally.

In some cases, there may be no gradient defined for the lookup of the nearest neighbor in the codebook of embeddings for each latent embedding as the operation is non-differentiable. Instead, the lookup of the nearest neighbor in the codebook replaces each quantized latent embeddings z_{q}(x) with one of the learned codebook embeddings having the same dimensions. A stop-gradient (sg) operation may copy the gradients from the quantized latent embeddings z_{q}(x), input into the decoder f_{d}(θ_{d}), to continuous latent embeddings zₑ(x) output by the encoder fₑ(θₑ) before quantization. The stop-gradient (sg) operation may act as an identity function in the forward direction by copying the variables without any change. However, during the backward pass, which updates the gradient of the encoder fₑ(θₑ), the stop-gradient (sg) operation may prevent the gradient from flowing through the gradient update for the specific term to which the operation is applied at least because no gradient can be computed for that term.

In some example embodiments, the training of the encoder fₑ and the decoder f_{d}, which forms an autoencoder (e.g., a variational autoencoder (VAE) and/or the like), may include adjusting the encoder fₑ and the decoder f_{d} to reduce (or minimize) three separate losses or loss terms. The first loss term may include the reconstruction loss (e.g., a mean-squared error (MSE) reconstruction loss) corresponding to a difference between the voxelized molecule representation x ingested by the encoder fₑ to generate the embedding zₑ and the reconstruction \hat{x} generated by the decoder f_{d} based on the embedding zₑ. The second loss term may enforce the learning of the codebook of embeddings e used to quantize the latent space by moving the embedding vector et towards the continuous latent embeddings zₑ(x) output by the encoder fₑ. The third loss term may quantify a commitment loss, which ensures that the encoder fₑ commits to an embedding zₑ(x) and its output does not grow arbitrarily. This third loss term may be associated with a commitment cost weight β, which may also be a hyperparameter that is set through experimentation. Equation (5) below is an example of the overall loss function L for training the encoder fₑ and the decoder f_{d}.

$$L = {‖ \hat{x} - x ‖}_{2}^{2} + {‖ sg [ {z}_{e} ( x ) ] - e ‖}_{2}^{2} + β {‖ {z}_{e} ( x ) - sg [ e ] ‖}_{2}^{2} \qquad ( 5 )$$

At 354, the molecule design engine 110 may generate an updated embedding by at least updating the embedding of the three-dimensional representation of the input molecule. In some example embodiments, the molecule design engine 110 may apply the molecule design computation model 115 (e.g., the denoising model 117), to denoise the three-dimensional representation of the input molecule 152 by at least on updating the embedding 154 of the three-dimensional representation of the input molecule 152 and generating the updated embedding 156. For example, in some cases, the three-dimensional representation of the input molecule 152 may include noise that contribute to inconsistencies between the types and/or positions of atoms present in the input molecule 152 and those of the atoms in molecules that exhibit the one or more desired properties (e.g., drug-like properties). In other words, the molecule design computation model 115 may update the embedding 154 of the three-dimensional representation of the input molecule 152 in order to increase the likelihood of the resultant output molecule 162 exhibiting the one or more desired properties. As noted, the noise that is being removed from the embedding 154 by the denoising model 117 should not be conflated with the noise that projects the three-dimensional representation of the input molecule 152 from its true data distribution, which exhibits jagged density transitions, to a noisy data distribution exhibiting smoother density transitions for more efficient sampling (e.g., gradient-based Markov Chain Monte Carlo (MCMC) sampling and/or the like) therefrom. As described in more details below, by updating the embedding 154 of the three-dimensional representation of the input molecule 152, the molecule design computation model 115 (e.g., the denoising model 117) may traverse the smoother densities of the noisy data distribution to sample the updated embedding 156 from incrementally higher density regions of the noisy data distribution before “jumping” back to the true data distribution when a sample exhibiting a threshold likelihood of being within the noisy data distribution is selected.

In some example embodiments, the denoising model 117 may apply, to the embedding 154 of the three-dimensional representation of the input molecule 152, updates that correspond to changing the types and/or positions of the atoms present in the input molecule 152. In instances where the encoder 111 implements an identity function and the embedding 154 is generated without any down sampling (or compression) of the underlying three-dimensional representation (e.g., voxelized representation) of the input molecule 152, the denoising model 117 may update the embedding 154 by at least updating the atomic density of one or more voxels in at least one channel of the embedding 154. Alternatively, in cases where the generating of the embedding 154 includes down sampling (or compression) of the underlying three-dimensional representation (e.g., voxelized representation) of the input molecule 152, the denoising model 117 may update the embedding 154 by at least updating one or more values present in the embedding 154, at least some of which condensing multiple atomic density values included in the three-dimensional representation (e.g., voxelized representation) of the input molecule 152.

In some example embodiments, the molecule design computation model 115 may apply the denoising model 117 to update the embedding 154 of the input molecule 152 based on the function 175. In some cases, the function 175 may output, for each sample (or molecule) selected from the noisy data distribution, a value (e.g., a score and/or the like) indicative of the likelihood of the sample (or molecule) being in the noisy data distribution. For example, in some cases, the value output by the function 175 for a particular sample (or molecule) may indicate the local change in density at the location from which the sample (or molecule) is selected. The denoising model 117 may update, based at least on the values output by the function 175, the embedding 154 over multiple successive sampling iterations. During each sampling iteration, the denoising model 117 may be applied to further update the embedding 154 such that the resulting updated embedding 156 is selected from a higher density region of the noisy data distribution than in previous sampling iterations.

In some example embodiments, the molecule design computation model 115 may perform a gradient based Markov Chain Monte Carlo (MCMC) sampling (e.g., Langevin Markov Chain Monte Carlo (MCMC) sampling) of the noisy data distribution in which the embedding 154 of the three-dimensional representation (e.g., voxelized representation) of the input molecule 152 is updated over multiple successive sampling iterations, with each iteration sampling from an incrementally higher density region of the noisy data distribution to increase the likelihood of the resulting updated embedding 156 being in the noisy data distribution. Moreover, in some cases, the updates made to the embedding 154 of the input molecule 152 may be cumulative over the multiple successive iterations. To further illustrate, consider an example in which the embedding 154 of the three-dimensional representation of the input molecule 152 undergoes a first update and a second update. The molecule design computation model 115 may apply the function 175 to determine a first value (e.g., first score and/or the like) of the embedding 154 having the first update and a second value (e.g., second score and/or the like) of the embedding 154 having the second update. During a subsequent iteration of gradient-based Markov Chain Monte Carlo (MCMC) sampling, the denoising model 117 may be applied to further update the embedding 154 having the first update if the first value and the second value indicate that the embedding 154 having the first update is sampled from a higher density region of the noisy data distribution and exhibits a higher likelihood of being within the noisy data distribution than the embedding 154 having the second update. In some cases, one or more additional iterations of the gradient-based Markov Chain Monte Carlo (MCMC) sampling may be performed, with the molecule design computation model 115 applying the denoising model 117 to further modify the embedding 154 of the three-dimensional representation (e.g., voxelized representation) of the input molecule 152, until one or more criteria are met. For instance, in some cases, the molecule design computation model 115 may perform one or more additional iterations of gradient based Markov Chain Monte Carlo (MCMC) sampling until a threshold quantity of sampling iterations are performed. Alternatively and/or additionally, the molecule design computation model 115 may perform one or more additional iterations of gradient based Markov Chain Monte Carlo (MCMC) sampling until the function 175 outputs, for the updated embedding 156, a value (e.g., score and/or the like) satisfying one or more thresholds. That the value (e.g., score and/or the like) associated with the updated embedding 156 satisfies the one or more thresholds may indicate that the updated embedding 156 is selected from a region of the noisy data distribution having a sufficiently high density and that the likelihood of the updated embedding 156 being within the noisy data distribution satisfies one or more thresholds. In some cases, the one or more criteria may also include having generated a threshold quantity of output molecules exhibiting the one or more desired properties (e.g., at least one output molecule exhibiting a threshold level of one or more drug-like properties such as affinity, specificity, biological activity, developability, and/or the like).

At 356, the molecule design computation model 115 may decode the updated embedding to generate a noisy three-dimensional representation of an output molecule. In some example embodiments, the molecule design engine 110 may, upon having applied the molecule design computation model 115 (e.g., the denoising model 117) to update the embedding 154 of the three-dimensional representation of the input molecule 152 and generate the updated embedding 156, apply the decoder 119 to decode the updated embedding 156 and generate the noisy three-dimensional representation 158 of the output molecule 162. The decoding of the updated embedding 156 may map the updated embedding 156 from the latent voxelized space, which is populated by embeddings of the three-dimensional representations of various molecules, to the latent discrete space. However, as described in more details below, the latent discrete space may be a noisy latent space, meaning that the noisy three-dimensional representation 158 generated by the decoder 119 decoding the updated embedding 156 may require further denoising in order to project the noisy three-dimensional representation 158 back to the true data distribution of molecules exhibiting the one or more desired properties.

In some example embodiments, the decoder 119 of the molecule design engine 110 may generate the noisy three-dimensional representation 158 by at least decoding the updated embedding 156 generated by the molecule design computation model 115 (e.g., the denoising engine 117). As noted, in some case, the decoder 119 may, along with the encoder 111, form a part of an autoencoder (e.g., a variational autoencoder such as a vector quantized variational autoencoder (VQ-VAE)). In some cases, the encoder 111 and the decoder 119 may be trained in tandem, with the encoder 111 may trained to generate embeddings of the three-dimensional representations (e.g., voxelized representations) of molecules, such as the embedding 154 of the three-dimensional representation of the input molecule 152, that enable the decoder 119 to recover the original three-dimensional representations (e.g., voxelized representation) therefrom. Accordingly, upon generating the updated embedding 156, the decoder 119 may be applied to recover the noisy three-dimensional representation 158 of the output molecule 162 therefrom.

In some cases, the decoding of the updated embedding 156 may include upsampling (or decompressing) the updated embedding 156, which may project the updated embedding 156 from the latent voxelized space back to the discrete voxelized space. The noisy three-dimensional representation 158 (e.g., noisy voxelized representation) of the output molecule 162 may exhibit the same dimensionality (or quantity of features) as the three-dimensional representation (e.g., voxelized representation) of the input molecule 152 ingested by the molecule design engine 110 at operation 352. For example, in some cases, the three-dimensional representation of the input molecule 162 may include a [32×32×32] voxel grid, meaning that the three-dimensional representation of the input molecule 152 may include 32,000 features (or atomic density values). Meanwhile, each of the embedding 154 that the molecule design computation model 115 operates upon and the resulting updated embedding 156 may include a [4×4×4] voxel grid having 64 features. In some cases, the decoder 119 may decode the updated embedding 156 by upsampling (or decompressing) the [4×4×4] voxel grid included therein to generate a [32×32×32] voxel grid for the noisy three-dimensional representation 158 (e.g., noisy voxelized representation) of the output molecule 162. It should be appreciated that this upsampling (or decompressing) may restore the 32,000 features (or atomic density values) that are in the noisy three-dimensional representation 158 (e.g., voxelized representation) of the output molecule 162. As noted, these 32,000 features (or atomic density values) may indicate the positions of various atoms present in the output molecule 162. Moreover, the 32,000 features (or atomic density values) may span one or multiple channels, each of which corresponding to a type of atoms that may be present in the output molecule 162.

At 358, the molecule design engine 110 may denoise the noisy three-dimensional representation of the output molecule to generate a three-dimensional representation of the output molecule. In some example embodiments, the denoising engine 117 of the molecule design computation model 115 may generate the three-dimensional representation (e.g., voxelized representation) of the output molecule 162 by at least denoising the noisy three-dimensional representation 158 generated by the decoder 119 decoding the updated embedding 156. As noted, in some cases, the molecule design computation model 115 (e.g., the denoising model 117) may generate the updated embedding 156 over one or more iterations of gradient-based Markov Chain Monte Carlo (e.g., Langevin Markov Chain Monte Carlo and/or the like). In doing so, the molecule design computation model 115 may traverse a noisy latent distribution, based at least on the output of the function 175 (e.g., the score output by the function 175), in order to sample the updated embedding 156 from a higher density region of the noisy latent distribution populated by embeddings of three-dimensional representations of molecules more likely to exhibit the one or more desired properties (e.g., drug-like properties). However, decoding the updated embedding 156 merely maps the updated embedding 156 from the latent voxelized space to the discrete voxelized space but the noisy three-dimensional representation 158 still occupies a noisy data distribution and not the true data distribution of molecules exhibiting the one or more desired properties. As such, in some cases, the recovery model 118 may be applied to map the noisy three-dimensional representation 158 from the noisy data distribution to the true data distribution. In some cases, this may constitute a “jump” back to the true data distribution, meaning that the three-dimensional representation of the output molecule 162 generated therefrom occupies the true data distribution.

In some cases, the recovery model 118 may share a same architecture (e.g., an artificial neural network (ANN) and/or the like) as the denoising model 117 trained to traverse the noisy latent distribution to denoise the embedding 154 of the three-dimensional representation of the input molecule 152 and generate the updated embedding 156. However, as noted, the recovery model 118 may be trained to remove a different type of noise. Accordingly, in some cases, the recovery engine 118 may be trained based on the training dataset to denoise the noisy three-dimensional representation 182 of the sample molecule and recover the original three-dimensional representation 182 therefrom. Contrastingly, the denoising engine 117 may be trained to recover, from the corrupted embedding 188, the embedding 186 of the noisy three-dimensional representation 182 of the sample molecule. In this context, the training of the denoising engine 117 may include adjusting one or more parameters of the denoising engine 117 (e.g., the artificial neural network (ANN) and/or the like) to reduce (or minimize) the difference (e.g., mean squared error (MSE)) between the original three-dimensional representation of the sample molecule and the three-dimensional representation of the sample molecule that the denoising engine 117 recovers from the noisy three-dimensional representation of the sample molecule.

To further illustrate, consider the quantized latent embeddings z_{q}(x) described in operation 252. As noted, the quantized latent embeddings z_{q}(x) may be generated by the encoder fₑ(x) encoding the voxelized molecule representations x. In some cases, noise ε (e.g., Gaussian noise such as isotropic Gaussian noise) may be added to the quantized latent embeddings z_{q}(x). For example, in some cases, noise ε (e.g., Gaussian noise such as isotropic Gaussian noise) with identity covariance matrix scaled by a fixed large noise level σ may be added in accordance with Equation (6) below.

$$y = {z}_{q} ( x ) + ϵ , ϵ ∼ N ( 0 , {σ}^{2} {I}_{d} ) \qquad ( 6 )$$

The denoising engine 117, which may be denoted as the latent model ζ(φ), may be trained to denoise and recover the latent embeddings z_{q}(x) while reducing (or minimizing) the reconstruction loss (e.g., a mean-squared error (MSE) reconstruction loss) between the original latent embeddings z_{q}(x) prior to (or without) the addition of noise E and the denoised latent embeddings \hat{z}_{q}(x) generated by the denoising engine 117. The denoising that is performed by the latent model ζ(φ) to generate the denoised latent embeddings \hat{z}_{q}(x) is shown in Equation (7) below. Meanwhile, Equation (8) shows the loss function L for training the latent model ζ(φ), which includes reducing (or minimizing) the difference (e.g., mean-squared error (MSE)) between the original latent embeddings z_{q}(x) prior to (or without) the addition of noise ε and the denoised latent embeddings \hat{z}q(x).

$$ζ ( ϕ ) : y \to {\hat{z}}_{q} ( x ) \qquad ( 7 )$$

$$L = {‖ {z}_{q} ( x ) - {\hat{z}}_{q} ( x ) ‖}_{2}^{2} \qquad ( 8 )$$

At 360, the molecule design engine 110 may generate, based at least on the three-dimensional representation of the output molecule, one or more other representations of the output molecule. In some example embodiments, the molecule design engine 110 may generate, based at least on the voxelized representation of the output molecule 162, one or more other representations of the output molecule 162 including, for example, a one-dimensional representation (e.g., a simplified molecular-input line-entry system (SMILES) string) of the output molecule 162, a two-dimensional representation (e.g., a molecular graph) of the output molecule 162, and/or the like. For example, in some cases, the molecule design computation model 110 may recover, from the voxelized representation of the output molecule 162, the positions (e.g., coordinates) of the atoms present in the output molecule 162 and the bonds therebetween. In some cases, the molecule design engine 110 may apply a peak detection technique, which determines the positions (e.g., coordinates) of the atoms present in the output molecule 162 based on one or more peaks in the atomic densities included in the voxelized representation of the output molecule 162 before determining, based on the positions of the atoms, one or more interconnecting bonds. Alternatively, the molecule design engine 110 may apply a machine learning model trained to translate the voxelized representation of the output molecule 162 into one or more other representations.

As noted, in some example embodiments, the molecule design computation model 115, including the denoising model 117, may operate on three-dimensional representations of molecules, instead of one- or two-dimensional representations of molecules, at least because realistic and valid molecules exhibiting certain desired properties are more likely to be generated based a representation of molecules that captures the composition (e.g., constituent atoms) as well as the conformation (or three-dimensional structure) of the molecules. In some cases, the molecule design computation model 115, including the denoising model 117, may operate on voxelized representations of molecules. Unlike conventional three-dimensional representations of molecules (e.g., point-cloud representation and/or the like), voxelized representations of molecules may jointly represent the atomic types and positions as one or more continuous (e.g., Gaussian-like) distributions across voxel grids that are centered around the atomic coordinates of individual atoms. Accordingly, unlike conventional three-dimensional representations of molecules (e.g., point-cloud representation and/or the like), the molecule design computation model 115 may apply the denoising model 117 to operate on the voxelized representation of an input molecule without requiring any workarounds to reconcile different types of data distributions (e.g., discrete distribution for atom types and continuous distribution for atomic position) and without any a priori knowledge of the number of atoms present in the output molecule resulting therefrom.

To further illustrate, FIG. 4 depicts examples of the voxelized representation of different molecules and the corresponding two dimensional representations, in accordance with some example embodiments. For example, FIG. 4 shows the voxelized representation 400 as well as the two-dimensional representation 450 of a molecule. In some example embodiments, the voxelized representation 400 of the molecule may be generated by partitioning (or discretizing) the three-dimensional space around the constituent atoms into a voxel grid 410, with each type of atom (or element) present in the molecule being represented by a different grid channel. This partitioning (or discretization) may generate n voxelized molecules

$${\{ {x}_{i} \}}_{i = 1}^{n} ,$$

xᵢ∈R^{d}, d=c×l³, wherein l denotes the length of each grid edge and c denotes the number of channels (e.g., quantity of different types of atoms (or elements)) in the dataset.

In some cases, the voxel grid 410 may be a three-dimensional grid of voxels organized into contiguous layers of rows and columns. Each voxel in the voxel grid 410 may be a volume element, such as a three-dimensional cube, formed at the intersection of a row and a column. Moreover, each voxel in the voxel grid 410 may be associated with a value (e.g., having a value [0,1]) indicative of the atomic density at the corresponding location. For a single molecule, the corresponding voxelized representation may be a box around the center of the molecule that is then divided into voxels. To generate the voxelized representation 400 of the molecule, each constituent atom may be converted into three-dimensional continuous (e.g., Gaussian-like) densities in accordance with Equation (9) below. For instance, the example of the voxel grid 410 shown in FIG. 4 may include a first atomic density 415a representative of a first atom of a first type and a second atomic density 415b representative of a second atom of a second type.

$${V}_{α} ( d , {r}_{α} ) = exp exp ( - \frac{{d}^{2}}{{( \text{.93} \cdot {r}_{α} )}^{2}} ) \qquad ( 9 )$$

wherein V_{α} is defined as a fraction of occupied volume by an atom α having a radius r_{α} at a distance d from the center of the atom. Different types of atoms (or elements) may have different radii or the same radius (e.g., r_{α}=0.5 Å). According to Equation (10) below, the occupancy of O_{cc} of each voxel in the voxel grid may be computed by integrating the occupancy generated by every atom in the molecule.

$${O}_{c {c}_{i , j , k}} = 1 - {\prod }_{n = 1}^{{N}_{α}} ( 1 - {V}_{{α}_{n}} ( ‖ {C}_{i , j , k} - {x}_{n} ‖ , {r}_{{α}_{n}} ) ) \qquad ( 10 )$$

wherein N_{α} denotes the number of atoms in the molecule, αₙ is the nth atom, C_{i,j,k} are the coordinates (i, j, k) in the voxel grid, and xₙ denotes the coordinates of the center of the atom n.

As noted, in some cases, the atomic densities in the voxelized representation 400 of the molecule may be centered around the atoms present in the molecule. Accordingly, the occupancy O_{cc} may take a maximum value (e.g., a value of 1) at the center of the atom and diminishes to a minimum value (e.g., a value of 0) as the distance from the center of the atom increases. Every channel in the voxel grid may be independent. That is, the channels do not interaction or share volumetric contributions. In some cases, the size of the voxel grid 410 included in the voxelized representation 400 of the molecule may correspond to the size of the molecule (e.g., the quantity of constituent atoms) being represented. For example, in some cases, the voxel grid 410 may be a [32×32×32] voxel grid if the molecule has fewer atoms (e.g., the QM9 molecule dataset) or a [64×64×64] voxel grid if the molecule has more atoms (e.g., the Geometric Ensemble of Molecules (GEOM) Drugs dataset). Moreover, in some cases, the number of channels in the voxelized representation 400 of the molecule may correspond to the number of atom types (or elements) present in the molecule. For instance, the voxelized representations of molecules in QM9 molecule dataset may include five channels for the five types of atoms forming those molecules (e.g., carbon (C), hydrogen (H), oxygen (O), nitrogen (N), and fluorine (F)). Meanwhile, the voxelized representations of the molecules in the Geometric Ensemble of Molecules (GEOM) Drugs dataset may include eight channels for the eight types of atoms present in those molecules (e.g., carbon (C), hydrogen (H), oxygen (O), nitrogen (N), fluorine (F), sulfur (S), chlorine (Cl), and bromine (Br)). Accordingly, the voxelized representation of each molecule in the QM9 molecule dataset may include a R^{5×32×32×32} voxel grid while the voxelized representation of each molecule in the Geometric Ensemble of Molecules (GEOM) Drugs dataset may include a R^{8×64×64×64} voxel grid.

As noted, in some example embodiments, the molecule design computation model 115, including the denoising model 117, may be trained to approximate and subsequently sample from a noisy data distribution of noisy voxelized representations of molecules or, in some cases, noisy embeddings of the voxelized representations of molecules, instead of the true data distribution of the voxelized representations of molecules that have not been perturbed with any noise. Training the denoising model 117 to approximate the noisy data distribution of molecules, such as the noisy data distribution of noisy voxelized representations of molecules exhibiting certain desired properties (e.g., drug-like properties) or noisy embeddings thereof, may include determining the function 175 such that the function 175 outputs, for the voxelized representation of each molecule (or the noisy embedding thereof) sampled from the noisy data distribution, a value indicative of the density of corresponding locations in the noisy data distribution. In instances where the function 175 is a score function, the function 175 may output a score corresponding to the local change in density (or gradient) of the noisy data distribution. Accordingly, in instances where the function 175 is a score function, the score output by the function 175 for the noisy voxelized representation of a molecule (or a noisy embedding thereof) may indicate the local change in density at the corresponding location in the noisy data distribution.

In some cases, the denoising engine 117 may be trained to denoise the noisy voxelized representations of molecules or, in some cases, the noisy embeddings of the voxelized representations of molecules, generated by the molecule design computation model 115 (e.g., the denoising model 117). To further illustrate, FIG. 5A depicts a schematic diagram illustrating an example of training the denoising engine 117 to denoise noisy voxelized representations of molecules, in accordance with some example embodiments. As shown in FIG. 5A, the training dataset for training the denoising engine 117 may be generated to include multiple training samples, each of which corresponding to a sample molecule. For example, FIG. 5A shows a sample molecule 500, which may be a known molecule from the PubChem dataset, the QM9 molecule dataset, the Geometric Ensemble of Molecules (GEOM) Drugs dataset, and/or the like. The sample molecule 500 may be rendered in a one-dimensional representation (e.g., a simplified molecular-input line-entry system (SMILES) string) or a two-dimensional representation (e.g., a molecular graph), neither of which adequately capture the conformation (or three-dimensional structure) of the sample molecule 500. Accordingly, in some cases, in order to generate a training sample for inclusion in the training dataset, the one- or two-dimensional representation of the sample molecule 500 may be translated into a three-dimensional representation of the sample molecule 500. For instance, in some cases, the one- or two-dimensional representation of the sample molecule 500 may be translated into the voxelized representation xᵢ shown in FIG. 5A. The voxelized representation xᵢ of the sample molecule 500 may jointly represent the types and positions of the atoms present in the sample molecule 500 as one of more continuous (e.g., Gaussian-like) densities across a voxel grid, centered around the individual atoms present in the sample molecule 500.

Referring again to FIG. 5A, in some cases, the voxelized representation xᵢ of the sample molecule 500 may be adulterated with noise € (e.g., Gaussian noise such as isotropic Gaussian noise and/or the like), which may have a noise level σ, in order to generate the noisy voxelized representation yᵢ. The addition of noise e may project the voxelized representation xᵢ from a true data distribution p(x) populated by clean (or original) voxelized representations of molecules to a noisy data distribution p(y) populated noisy voxelized representations of molecules. As noted, if the molecule design computation model 115 operates directly on clean (or original) voxelized representations of molecules from the true data distribution p(x), such as the voxelized representation xᵢ of the sample molecule 500, the jagged energy landscape of the true data distribution p(x) may prevent the molecule design computation model 115 from adequately exploring the true data distribution p(x) when sampling therefrom. Contrastingly, the noisy data distribution p(y) may exhibit a smoother energy landscape with more gradual gradient changes, meaning that the molecule design computation model 115 may sample from the noisy data distribution p(y) to yield greater diversity in the resulting output molecules. Accordingly, in some cases, the denoising engine 117 may be trained to denoise noisy voxelized representations of molecules, such as the noisy voxelized representation xᵢ of the sample molecule 500, such that the denoising engine 117 may be applied to denoise the noisy voxelized representations of molecules generated by the molecule design computation model 115 sampling from the noisy data distribution p(y). As described in more details below, in some cases, the voxelized representation xᵢ of the sample molecule 500 may undergo down sampling (or compression) before the addition of noise E, meaning that the denoising engine 117 may be trained to denoise the noisy embeddings of the voxelized representations of molecules instead of the noisy voxelized representations of molecules shown in FIG. 5A.

Referring again to FIG. 5A, the denoising engine 117 may be trained to denoise the noisy voxelized representation yᵢ. In some cases, the denoising engine 117 may be trained to denoise the noisy voxelized representation yᵢ by at least recovering the corresponding clean voxelized representation xᵢ therefrom. For example, in some cases, the denoising engine 117 may be an encoder-decoder three-dimensional convolutional neural network (CNN) trained to map the noised voxels in the noisy voxelized representation yᵢ to a corresponding clean voxel. In doing so, the denoising engine 117 may generate a denoised voxelized representation \hat{x}(yᵢ) that approximates the clean voxelized representation xᵢ. For instance, in some cases, the training of the denoising engine 117 may including adjusting the parameters of the denoising engine 117 to reduce (or minimize) a difference (e.g., mean-squared error (MSE)) between the denoised voxelized representation \hat{x}(yᵢ) and the corresponding clean voxelized representation xᵢ. In some cases, the noise level σ, which determines the quantity of noise ε added to the voxelized molecule representations xᵢ, may be set as a hyperparameter of the denoising engine 117. Moreover, in some cases, the noise level σ may be kept fixed (or constant) during the training of the denoising engine 117, which reduces the complexity of the training process compared to diffusion models. It should be appreciated that single-step denoising (as opposed to diffusion over multiple timesteps) may be sufficient to reconstruct the original voxelized representation xᵢ due to the nature of the voxelized representation xᵢ which, unlike natural images, contains more structural information on the sample molecule 500 than textural information.

In some example embodiments, the molecule design computation model 115 may apply the denoising model 117 to generate a voxelized representation of an output molecule by at least denoising the noisy voxelized representation of an input molecule over one or more iterations of gradient-based Markov Chain Monte Carlo (MCMC) sampling (e.g., Langevin Markov Chain Monte Carlo (MCMC) sampling and/or the like). In some cases, the denoising model 117 may sample from the noisy data distribution p(y), which includes traversing the noisy data distribution p(y) towards incrementally higher density regions of the noisy data distribution p(y), which are populated by molecules exhibiting one or more desired properties (e.g., drug-like properties). To further illustrate, FIG. 5B shows that the traversal across the noisy data distribution p(y) include selecting samples (or molecules) yₖ₋₁ at sampling iteration k−1, yₖ at sampling iteration k, and yₖ₊₁ at sampling iteration k+1. In some cases, the traversal of the noisy data distribution p(y) may be guided by the function 175 such that the sample yₖ is sampled from a higher density region of the noisy data distribution p(y) than sample yₖ₋₁ while sample yₖ₊₁ is sampled from an even higher density region of the noisy data distribution p(y) than sample yₖ. In some cases, each iteration of gradient-based Markov Chain Monte Carlo (MCMC) may include further modifying the sample (or molecule) selected during a previous iteration. Accordingly, as shown below, the sample yₖ₊₁ selected from the noisy data distribution p(y) during sampling iteration k+1 may be generated based on the sample yₖ selected during the previous sampling iteration k. Equation (10) below expresses the traversal of the noisy data distribution p(y).

$${dv}_{t} = - γ {v}_{t} dt - {ug}_{θ} ( {y}_{t} ) dt + ( \sqrt{2 γ u} ) d {B}_{t} {dy}_{t} = {v}_{t} dt , \qquad ( 10 )$$

wherein Bₜ denotes the standard Brownian motion in R^{d}, and γ and μ are hyperparameters (friction and inverse mass, respectively). A discretization technique, an example of which is shown as Algorithm 1 in Table 1 below, may be applied to generate the samples yₖ, which includes a discretization step δ.

Referring again to FIG. 5B, in some cases, the voxelized representation \hat{x}(yₖ) of a molecule may be generated when the denoising engine 117 denoises a corresponding noisy voxelized representation yₖ selected from the noisy data distribution p(y). As noted, the denoising of the noisy voxelized representation yₖ may project the noisy voxelized representation yₖ back to the true data distribution p(x), for example, by applying the least squares estimator σ²∇_{y} log log p(y). This constitutes the “jump” shown in FIG. 5B. Furthermore, in the example shown in FIG. 5B, a “jump” back to the true data distribution p(x) may be performed at each sampling iteration while the denoising model 117 is applied to traverse the noisy data distribution p(y) and select samples therefrom. For example, the molecule \hat{x}ₖ₋₁ may be generated when the sample yₖ₊₁ selected from the noisy data distribution p(y) during sampling iteration k+1 is denoised and projected back to the true data distribution p(x) while the molecule \hat{x}ₖ may be generated when the sample yₖ selected from the noisy data distribution p(y) during the subsequent sampling iteration k+1 is denoised and projected back to the true data distribution p(x).

TABLE 1
Algorithm 1:
Walk-jump sampling using the discretization of Langevin diffusion.

| | |
|---|---|
| 1: | Input δ (step size), u (inverse mass), γ (friction), K (steps taken) |
| 2: | Input Learned score function g_{θ}(y) ≈ ∇_{y} log log p(y) and noise |
| | level σ |
| 3: | Output \hat{x}_{K} |
| 4: | y₀~N(0, σ²I_{d}) + U_{d}(0,1) |
| 5: | v₀ ← 0 |
| 6: | For k = 0, ... , K − 1 do |
| 7: | ${y}_{k + 1} ← {y}_{k} + \frac{δ}{2} {v}_{k}$ |
| 8: | g ← g_{θ}(yₖ₊₁) |
| 9: | ${v}_{k + 1} ← {v}_{k} + \frac{u δ}{2} g$ |
| 10: | ε~ N(0, I_{d}) |
| 11: | ${v}_{k + 1} ← exp exp ( - γδ ) {v}_{k + 1} + \frac{u δ}{2} g + \sqrt{u ( 1 - exp exp ( - 2 γδ ) ) ε}$ |
| 12: | ${y}_{k + 1} ← {y}_{k + 1} + \frac{δ}{2} {v}_{k + 1}$ |
| 13: | end for |
| 14: | \hat{x}_{K} ← y_{K} + σ²g_{θ}(yₖ) |

Lines 6-13 correspond to the traversing of the noisy data distribution p(y) and the sampling therefrom while line 14 corresponds to the denoising operation.

In some example embodiments, the denoising model 117 may continue to traverse the noisy data distribution p(y) and select samples therefrom until one or more criteria are met. For example, the denoising model 117 may continue traversing the energy landscape of the noisy data distribution until the sampling iteration k+1 if a threshold quantity of sampling iterations are performed at that point. Alternatively and/or additionally, the denoising model 117 may continue traversing the energy landscape of the noisy data distribution p(y) until the sample yₖ₊₁ is selected if the sample yₖ₊₁ exhibits a threshold likelihood of being in the noisy data distribution p(y). To further illustrate, FIG. 5C shows the denoising model 117 being applied to select multiple successive samples from the noisy data distribution p(y) including, for example, samples 510a through 510f. In the example shown in FIG. 5C, the sampling (e.g., gradient-based Markov Chain Monte Carlo (MCMC) sampling) may start with the molecule design computation model 115 applying the denoising model 117 to select a first sample y₀ from the noisy data distribution p(y). In some cases, the selecting of the first sample y₀ may include the denoising model 117 updating the noisy voxelized representation of a corresponding molecule (or a noisy embedding thereof).

As shown in FIG. 5C, the first sample y₀ may be denoised to generate the corresponding voxelized representation \hat{x}(y₀). This denoising operation may constitute a “jump” from the noisy data distribution p(y) back to the true data distribution p(x). Each subsequent sampling iteration may include the denoising model 117 being applied to further update the noisy voxelized representation of a molecule selected during a previous sampling iteration. In the example shown in FIG. 5C, the molecule design computation model 115 may continue applying the denoising model 117 until k successive samples have been selected from the noisy data distribution p(y). The k-th sample yₖ may be denoised, for example, by the denoising engine 117, to generate the corresponding voxelized representation \hat{x}(yₖ). Doing so may project the k-th sample yₖ from the noisy data distribution p(y) back to the true data distribution p(x). It should be appreciated that the value of k may determine the quantity of sampling iterations and the quantity of samples selected from the noisy data distribution p(y). Increasing the value of k may increase the updates performed to the initial input molecule (e.g., the “seed” molecule). A higher value for k may increase the difference between the initial input molecule (e.g., the “seed” molecule) and the final output molecule, as well as the novelty of final output molecule.

In some example embodiments, upon selecting the k-th sample yₖ from the noisy data distribution p(y) and denoising the k-th sample yₖ to generate the corresponding voxelized representation \hat{x}(yₖ), the molecule design engine 110 may generate one or more other representations based on the voxelized representation \hat{x}(yₖ). For example, in some cases, the molecule design engine 110 may generate, based at least on the voxelized representation \hat{x}(yₖ), a one-dimensional representation (e.g., a simplified molecular-input line-entry system (SMILES) string) and/or a two-dimensional representation (e.g., a molecular graph) of the corresponding molecule.

FIG. 5D depicts a schematic diagram illustrating an example of a process for generating other molecular representations from the voxelized representation \hat{x}(yₖ), in accordance with some example embodiments. In the example shown in FIG. 5D, the molecule design engine 110 may determine the atoms present in the corresponding molecule by at least identifying peaks (e.g., atomic density values satisfying one or more thresholds) in the voxelized representation \hat{x}(yₖ). Furthermore, the molecule design engine 110 may determine one or more bonds interconnecting the atoms present in the molecule. A one- or two-dimensional representation of the molecule may be generated based at least on the atoms and interconnecting bonds. Alternatively, in some cases, the molecule design engine 110 may apply a machine learning model trained to translate the voxelized representation \hat{x}(yₖ) into one or more other representations of the corresponding molecule.

In some example embodiments, instead of the operating in the noisy discrete voxelized space, for example, in the manner shown in FIGS. 5A-5D, the molecule design computation model 115 may operate in a noisy latent voxelized space. For example, in some cases, instead of the molecule design computation model 115 applying the denoising model 117 to denoise the noisy voxelized representation yᵢ, the denoising model 117 may be applied to denoise a noisy embedding of the voxelized representation xᵢ. To further illustrate, FIG. 6 depicts a schematic diagram illustrating an example of a process in which the molecule design computation model 115 generates voxelized representations of molecules by operating in the noisy latent voxelized space, in accordance with some example embodiments. Referring to FIG. 6, an input molecule 600, which may be rendered in a one- or two-dimensional representation, may be translated into the three-dimensional representation of the input molecule 152. In some cases, the three-dimensional representation of the input molecule 152 may be a voxelized representation of the input molecule 152, which jointly represents the types and positions of the atoms in the input molecule 152 as one or more continuous distribution of atomic densities across voxel grids. In some cases, instead of applying the denoising model 117 to operate directly on a noisy voxelized representation of the input molecule 152, the encoder 111 may first generate the embedding 154 of the voxelized representation of the input molecule 152 before noise ε is added to the embedding 154 of the voxelized representation of the input molecule 152. The resulting noisy embedding 156 may undergo one or more iterations of gradient-based Markov Chain Monte Carlo (MCMC) sampling (e.g., Langevin Markov Chain Monte Carlo (MCMC) sampling and/or the like). For example, each iteration of gradient-based Markov Chain Monte Carlo (MCMC) sampling may include the molecule design computation model 115 applying the denoising model 117 to denoise the noisy embedding 156 by at least updating the noisy embedding 156. As noted, updating the noisy embedding 156 in this manner may be tantamount to selecting one or more samples from a noisy data distribution populated by noisy embeddings of the voxelized representations of molecules exhibiting one or more desired properties. The sampling may be guided by the function 175 (e.g., a score function and/or the like) such that successive samples are selected from incrementally higher density regions of the noisy data distribution, which are more likely to be populated by noisy embeddings of the voxelized representations of molecules exhibiting the one or more desired properties.

Referring again to FIG. 6, the molecule design computation model 115 may generate the updated embedding 156 by at least updating, for example, over one or more iterations of gradient-based Markov Chain Monte Carlo (MCMC) sampling, the embedding 154 of the voxelized representation of the input molecule 152. As shown in FIG. 6, the embedding 154 may be denoised, for example, by the denoising engine 117, thereby generating the updated embedding 156. The denoising of the embedding 154 may including sampling, from the noisy latent distribution of molecules exhibiting the one or more desired properties, the updated embedding 156. Furthermore, as shown in FIG. 6, the decoder 119 may decode the updated embedding 156 to generate the voxelized representation of the corresponding output molecule 162. The decoding of the updated embedding 156 may project the updated embedding 156 from a latent voxelized space back to a discrete voxelized space. The resulting voxelized representation of the output molecule 162 may be further translated into a reconstructed molecule 650. It should be appreciated that the reconstructed molecule 650 may correspond to a one-dimensional representation (e.g., a simplified molecular-input line-entry system (SMILES) string) or a two-dimensional representation (e.g., a molecular graph) of the output molecule.

In some example embodiments, the generative performance of the molecule design computation model 115 may be evaluated based on a variety of metrics, some examples of which are described in Table 2 below.

TABLE 2

| Metric | Description |
|---|---|
| Atom | The percentage of generated atoms with the correct |
| Stability | valency. This metric may be computed on the raw three- |
| | dimensional sample (prior to any post processing) and is |
| | therefore a more stringent metric than validity. |
| Molecule | The percentage of generated molecules in which all |
| Stability | constituent atoms are stable. |
| Validity | The percentage of generated molecules that passes |
| | RDKit's sanitization filter. |
| Uniqueness | The proportion of valid molecules (defined above) with |
| | a unique canonical simplified molecular-input line-entry |
| | system (SMILES) string representation (generated with |
| | RDKit). |
| Atoms Total | The total variation between the distribution of bond types |
| Variation | in the generated and test set. Five atom types (elements) |
| (TV) | may be considered for the QM9 molecule dataset while |
| | eight atom types (elements) may be considered for the |
| | Geometric Ensemble of Molecules (GEOM) Drugs |
| | dataset. The histograms ĥₐₜₘ and hₐₜₘ are generated by |
| | counting the number of each atom type on all molecules |
| | in both the generated and real sample set. Atoms total |
| | variation may be computed as: |
| | $Atoms TV ( {\hat{h}}_{atm} , {h}_{atm} ) = {\sum }_{x ∈ atom types} \vert {\hat{h}}_{atm} ( x ) - {h}_{atm} ( x ) \vert$ |
| Bonds Total | The histograms ĥ_{bond} and h_{bond} for real and generated |
| Variation | samples may be generated by counting all bond types |
| (TV) | across all molecules. Bonds total variation may be |
| | computed as: |
| | $Bonds TV ( {\hat{h}}_{bond} , {h}_{bond} ) = {\sum }_{x ∈ bond types} \vert {\hat{h}}_{bond} ( x ) - {h}_{bond} ( x ) \vert$ |
| Valency W₁ | This metric is the weighted sum of the Wasserstein |
| | distance between the distribution of valencies for each |
| | atom type. Valency W₁ may be computed as: |
| | Valency W₁(generated, target) = |
| | E_{x∈atom types} p(x)W₁(ĥᵥₐₗ(x), hᵥₐₗ(x)), |
| | wherein ĥᵥₐₗ (x) and hᵥₐₗ (x) are the histograms of the |
| | valencies for atom type x for the generated and holdout |
| | set samples, respectively. |
| Bond | This metric is the weighted sum of the Wasserstein |
| Length W₁ | distance between the distribution of bond lengths for |
| | each bond type. Bond length W₁ may be computed as: |
| | Bond Len W₁(generated, target) = |
| | E_{x∈bond types} p(b)W₁(ĥ_{dist}(b), h_{dist}(b)), |
| | wherein ĥ_{dist}(b) and h_{dist}(b) are the histograms of bond |
| | lengths for bond type b for the generated and holdout set |
| | samples, respectively. |
| Bond | This metric is the weighted sum of the Wasserstein |
| Angle W₁ | distance between the distribution of bond angles (in |
| | degrees) for each atom type in the dataset. Bond angle |
| | W₁ may be computed as: |
| | Bond Ang W₁(generated, target) = |
| | E_{x∈atom types} p(x)W₁(ĥ_{ang}(x), h_{ang}(x)), |
| | wherein ĥ_{ang}(x) and h_{ang}(x) are the histograms of bond |
| | angles for atom type x for the generated and holdout set |
| | samples, respectively. |
| Strain | The strain energy for a generated molecule is computed |
| Energy | as the difference between the energy of a generated pose |
| | and the energy of a relaxed position, The relaxation and |
| | the energy may be computed using the UFF provided by |
| | RDKit. |

In some example embodiments, the generative performance of the molecule design computation model 115 may be dependent on one or more factors including, for example, the noise level σ, the difference in the number of sampling iterations Δk, and the radii of atomic density in the voxelized molecule representations. FIG. 7 depicts graphs illustrating the effect of the noise level σ on the stability and uniqueness (FIG. 7(a)), the atoms total variation and bonds total variation (FIG. 7(b)), and the valency W₁ and bond angle W₁ (FIG. 7(c)) of the molecules generated by the molecule design computation model 115 when different levels σ of noise ε (e.g., Gaussian noise such as isotropic Gaussian noise) is added to the voxelized representations of molecules operated upon by the molecule design computation model 115. As noted, unlike diffusion models, the noise level σ may be fixed during training and sampling, in accordance with various example embodiments described herein. Moreover, it should be appreciated that the noise level σ is a hyperparameter that imposes a tradeoff between the quality of the sampling (e.g., the gradient-based Markov Chain Monte Carlo (MCMC) sampling) and denoising (e.g., of the empirical Bayes framework). In some cases, the molecule design engine 110 may determine the noise level σ to correspond to the largest quantity of noise ε added to the voxelized representation of a molecule that the denoising engine 117 can still learn to denoise. For example, in some cases, the molecule design computation model 115 and the denoising engine 117 may be trained on the QM9 molecule dataset with varying noise levels σ={0.6, 0.7, . . . , 1.2}, while other hyperparameters are held constant. The graphs in FIG. 7(a),(b),(c) show that while some metrics improve at higher noise levels σ, molecule stability and valency W₁ deteriorate as the noise level σ increases. For the QM9 molecule dataset, the best overall performance across all metrics is achieved at a noise level σ of 0.9.

In some example embodiments, the number of sampling iterations Δk performed as part of gradient-based Markov Chain Monte Carlo (MCMC) may affect the novelty of the molecules generated by the molecule design computation model 115. This phenomenon is shown in FIG. 8, which depicts the molecules output by the molecule design computation model 115 (trained on the Geometric Ensemble of Molecules (GEOM) Drugs dataset) updating, over different numbers sampling iterations k, a noise molecule (for de novo generation) and a known molecule (for seeded generation). For example, FIG. 8 shows the voxelized representation of a first molecule 810 generated by the molecule design computation model 115 denoising a noise molecule (for de novo generation) over k=10 sampling iterations, the voxelized representation of a first molecule 820 generated by the molecule design computation model 115 denoising the noise molecule (for de novo generation) over k=50 sampling iterations, the voxelized representation of a third molecule 830 that generated by the molecule design computation model 115 denoising a noise molecule (for de novo generation) over k=100 sampling iterations, and/or the like.

In addition to the novelty of the molecules generated by the molecule design computation model 115, adjusting the number of sampling iterations k may also affect other aspects of the generative performance of the molecule design computation model 115. Table 3 below compares the generative performance of the molecule design computation model 115 at different numbers of sampling iterations Δk and that of the conventional generative model EDM, which performs 1,000 diffusion steps for generation. The results in Table 3 show that the molecule design computation model 115 performs better in some metrics as the number of or sampling iterations Δk increases. As expected, the average time consumed to generate each molecule (in seconds) increases linearly as the number of sampling iterations Δk increases. However, the molecule design computation model 115 remains faster than EDM even at 500 sampling iterations. Notably, at merely 50 sampling iterations, the molecule design computation model 115 already outperforms EDM in most metrics, while being an order of magnitude faster on average.

TABLE 3

| | stable | stable | | | | | | bond | bond | avg. |
|---|---|---|---|---|---|---|---|---|---|---|
| Δk | mol | atom | valid | unique | valency | atom | bond | len | ang | time |
| (n steps) | %_{↑} | %_{↑} | %_{↑} | %_{↑} | W_{1↓} | TV_{↓} | TV_{↓} | W_{1↓} | W_{1↓} | s/mol.↓ |
| 50 | 78.9 | 98.7 | 96.3 | 87.8 | .250 | .073 | .102 | .002 | 1.18 | 0.90 |
| 100 | 78.6 | 98.6 | 95.5 | 94.3 | .256 | .050 | .101 | .002 | 1.62 | 1.64 |
| 200 | 77.9 | 98.4 | 94.4 | 98.6 | .253 | .037 | .104 | .002 | 1.02 | 3.17 |
| 500 | 76.7 | 98.2 | 93.8 | 99.2 | .252 | .043 | .042 | .002 | 0.56 | 7.55 |
| 1000 | 75.5 | 98.4 | 93.4 | 99.8 | .257 | .029 | .050 | .002 | 0.79 | 14.9 |
| EDM | 40.3 | 97.8 | 87.8 | 99.9 | .285 | .212 | .048 | .002 | 6.42 | 9.35 |

In some example embodiments, the generative performance of the molecule design computation model 115 may also be impacted by the size of the atomic radii in the voxelized representations that the molecule design computation model 115 operates upon. It should be appreciated that the size of the atomic radii may change while the resolution of the voxel grid remains fixed (e.g., at 0.25 Å). The generative performance of the molecule design computation model 115, even with different hyperparameters, may peak at certain atomic radii. For example, when the molecule design computation model 115 is applied to operate on voxelized representations having atomic radii of 0.25, 0.5, 0.75, and 1.0, a fixed radius of 0.5 consistently outperformed the other values even as the hyperparameters of the molecule design computation model 115 changed.

In some example embodiments, the generative performance of the molecule design computation model 115 may be compared to existing generative models operating on conventional three-dimensional molecule representations such as GSchNet, a point-cloud autoregressive model, and EDM, a point-cloud diffusion-based model. Each model was applied to generate 10,000 samples, which were then evaluated based on the atom stability, molecule stability, validity, uniqueness, atoms total variation (TV), bonds total variation (TV), valency W₁, bond length W₁, and bond angle W₁. Table 4 below shows the results, with mean and standard deviation across three runs, for the samples generated by the molecule design computation model 115 (MDCM) trained on the QM9 molecule dataset. FIG. 9A depicts some examples of the voxelized representations of molecules generated by the molecule design computation model 115 trained on the QM9 molecule dataset as well as the corresponding molecular graphs. The cumulative distribution function (CDF) of the strain energies of the molecules generated by the molecule design computation model 115 trained on the QM9 molecule dataset, as compared to the molecules in the QM9 molecule dataset and those generated by the conventional generative model EDM, is shown in the graph 1000 depicted in FIG. 10A. FIG. 10B depicts a graph 1050 illustrating the empirical distribution of the number of atoms per molecule in the QM9 molecule dataset compared to the empirical distribution of the number of atoms in the molecules generated by the molecule design computation model 115 trained on the QM9 molecule dataset.

TABLE 4

| | stable | stable | | | | | | bond | bond |
|---|---|---|---|---|---|---|---|---|---|
| | mol | atom | valid | unique | valency | atom | bond | len | ang |
| | %_{↑} | %_{↑} | %_{↑} | %_{↑} | W_{1↓} | TV_{↓} | TV_{↓} | W_{1↓} | W_{1↓} |
| data | 98.7 | 99.8 | 98.9 | 99.9 | .001 | .003 | .000 | .000 | 120 |
| GSchNet | 92.0 | 98.7 | 98.1 | 94.5 | .049 | .042 | .041 | .005 | 1.68 |
| EDM | 97.9 | 99.8 | 99.0 | 98.5 | .011 | .021 | .002 | .001 | 0.44 |
| MDCM_{no rot} | 84.2_{(±1.6)} | 98.2_{(±.3)} | 98.1_{(±.4)} | 77.2_{(±1.7)} | .043_{(±.0)} | .171_{(±.200)} | .050_{(±.010)} | .007_{(±.0)} | 3.80_{(±.7)} |
| MDCM | 89.3_{(±.6)} | 99.2_{(±.1)} | 98.7_{(±.1)} | 92.1_{(±.3)} | .023_{(±.002)} | .029_{(±.009)} | .009_{(±.002)} | .003_{(±.002)} | 1.96_{(±.04)} |
| MDCM_{oracle} | 90.1 | 99.3 | 98.9 | 99.9 | .024 | .009 | .002 | .001 | 0.37 |

In some cases, the molecule design computation model 115 is also trained on the Geometric Ensemble of Molecules (GEOM) Drugs dataset before being applied to generate 10,000 samples. A comparison of those samples against the 10,000 samples generated by the conventional generative model EDM is shown in Table 5 below, with mean and standard deviation across three separate runs. FIG. 9B depicts some examples of the voxelized representations of molecules generated by the molecule design computation model 115 (MDCM) trained on the Geometric Ensemble of Molecules (GEOM) Drugs dataset as well as the corresponding molecular graphs. The cumulative distribution function (CDF) of the strain energies of the molecules generated by the molecule design computation model 115 (MDCM) trained on the Geometric Ensemble of Molecules (GEOM) Drugs dataset, as compared to the molecules in the GEOM Drugs dataset and those generated by the conventional generative model EDM, is shown in the graph 1100 depicted in FIG. 11A. FIG. 11B depicts a graph 1150 illustrating the empirical distribution of the number of atoms per molecule in the Geometric Ensemble of Molecules (GEOM) Drugs dataset compared to the empirical distribution of the number of atoms in the molecules generated by the molecule design computation model 115 (MDCM) trained on the GEOM Drugs dataset.

TABLE 5

| | stable | stable | | | | | | bond | bond |
|---|---|---|---|---|---|---|---|---|---|
| | mol | atom | valid | unique | valency | atom | bond | len | ang |
| | %_{↑} | %_{↑} | %_{↑} | %_{↑} | W_{1↓} | TV_{↓} | TV_{↓} | W_{1↓} | W_{1↓} |
| data | 99.9 | 99.9 | 99.8 | 100. | .001 | .001 | .025 | .000 | .050 |
| EDM | 40.3 | 97.8 | 87.8 | 99.9 | .285 | .212 | .048 | .002 | 6.42 |
| MDCM_{no rot} | 44.4_{(±.1)} | 96.6_{(±.1)} | 89.7_{(±.2)} | 99.9_{(±.0)} | .238_{(±.001)} | .025_{(±.001)} | .024_{(±.001)} | .004_{(±.000)} | 2.14_{(±.02)} |
| MDCM | 75.0_{(±1.)} | 98.1_{(±.3)} | 93.4_{(±.5)} | 99.1_{(±.2)} | .254_{(±.003)} | .033_{(±.041)} | .036_{(±.006)} | .002_{(±.001)} | 0.64_{(±.13)} |
| MDCM_{oracle} | 81.9 | 99.0 | 94.7 | 97.4 | .253 | .002 | .024 | .001 | 0.31 |

In cases where the molecule design computation model 115 (MDCM) is trained on the QM9 dataset, the molecule design computation model 115 showed comparable generative performance as the conventional generative model EDM. However, in cases where the molecule design computation model 115 (MDCM) is trained on the Geometric Ensemble of Molecules (GEOM) Drugs dataset, a more challenging and realistic drug-like dataset than the QM9 dataset, the molecule design computation model 115 outperformed EDM in eight out of nine metrics by a considerably large margin. For example, the molecules generated by the molecule design computation model 115 (MDCM) trained on the GEOM Drugs dataset showed significantly lower median strain energy than those generated by EDM. It can also be observed from the results in Tables 3 and 4 that augmenting the training dataset with rotations and translations improves the generative performance of the molecule design computation model 115 (e.g., MDCM_{no rot} versus MDCM). Overall, the molecule design computation model 115 is a more expressive model that scales better with data. In particular, the molecule design computation model 115 is more capable of capturing the many modes that are present in a large scale data distribution, such as the Geometric Ensemble of Molecules (GEOM) Drugs dataset.

FIG. 12A depicts a schematic diagram illustrating a comparison of seeded generation on Geometric Ensemble of Molecules (GEOM) Drugs at different sampling iterations in discrete voxelized space and latent voxelized space, in accordance with some example embodiments. Panel 1210 shows the molecular graphs of molecules generated at steps (or sampling iterations) 10, 20, 50, 100, and 200 by the molecule design computation model 115 operating in the latent voxelized space and updating an embedding of the voxelized representation of a seed molecule from the Geometric Ensemble of Molecules (GEOM) Drugs dataset. The corresponding voxelized representations of these molecules are shown in Panel 1220. Panel 1215 shows the molecular graphs of the molecules generated at steps (or sampling iterations) 5, 10, 50, 100, and 200 by the molecule design computation model 115 operating in the discrete voxelized space and updating the voxelized representation of a seed molecule from the Geometric Ensemble of Molecules (GEOM) Drugs dataset. The corresponding voxelized representations of these molecules are shown in Panel 1225. As shown in FIGS. 12A and B, whether operating in the latent voxelized space or the discrete voxelized space, the molecule design computation model 115 is able to generate stable, valid, and unique molecules that also closely resemble seed molecules from the Geometric Ensemble of Molecules (GEOM) Drugs dataset.

Table 6 below further depicts the seeded generation results (averaged over 5 repeats) on the Geometric Ensemble of Molecules (GEOM) Drugs dataset.

TABLE 6

| steps | tan | stable | stable | stable | | | | | bond | bond | |
|---|---|---|---|---|---|---|---|---|---|---|---|
| (sampling | sim. | mol | sanit. | atom | valid | valency | atom | bond | len | ang | avg. t |
| iterations) | %_{↑} | %_{↑} | %_{↑} | %_{↑} | %_{\hat{ }} | W_{1↓} | TV_{↓} | TV_{↓} | W_{1↓} | W_{1↓} | [s/mol] |
| 5 Discrete | 80.84 | 79.65 | 85.54 | 99.43 | 90.12 | 0.26 | 0.02 | 0.03 | 0.00 | 0.67 | 0.38 |
| 10 | 71.44 | 78.16 | 85.71 | 99.36 | 89.86 | 0.25 | 0.02 | 0.03 | 0.00 | 0.67 | 0.66 |
| 50 | 44.99 | 77.53 | 86.42 | 99.35 | 90.15 | 0.25 | 0.03 | 0.03 | 0.00 | 0.54 | 0.90 |
| 100 | 35.18 | 79.18 | 87.44 | 99.37 | 90.52 | 0.25 | 0.03 | 0.03 | 0.00 | 0.50 | 1.64 |
| 200 | 27.37 | 78.79 | 88.40 | 99.35 | 90.86 | 0.25 | 0.04 | 0.03 | 0.00 | 0.54 | 3.17 |
| 10 Latent | 88.47 | 81.19 | 85.73 | 99.46 | 90.29 | 0.26 | 0.03 | 0.03 | 0.00 | 0.77 | 0.21 |
| 20 | 84.3 | 80.14 | 85.46 | 99.42 | 89.82 | 0.26 | 0.03 | 0.03 | 0.00 | 0.85 | 0.23 |
| 50 | 63.85 | 72.43 | 83.11 | 99.16 | 86.61 | 0.25 | 0.03 | 0.03 | 0.00 | 1.15 | 0.28 |
| 100 | 38.33 | 55.91 | 79.52 | 98.45 | 81.42 | 0.23 | 0.04 | 0.03 | 0.00 | 1.80 | 0.36 |
| 200 | 20.18 | 31.6 | 77.08 | 96.74 | 77.96 | 0.20 | 0.07 | 0.04 | 0.00 | 3.56 | 0.52 |

FIG. 12B depicts a schematic diagram illustrating a comparison of seeded generation on PubChem drugs at different sampling iterations in discrete voxelized space and latent voxelized space, in accordance with some example embodiments. Panel 1450 shows the molecular graphs of molecules generated at steps (or sampling iterations) 10, 20, 50, 100, and 200 by the molecule design computation model 115 operating in the latent voxelized space and updating an embedding of the voxelized representation of a seed molecule from the PubChem dataset. The corresponding voxelized representations of these molecules are shown in Panel 1260. Panel 1255 shows the molecular graphs of the molecules generated at steps (or sampling iterations) 5, 10, 50, 100, and 200 by the molecule design computation model 115 operating in the discrete voxelized space and updating the voxelized representation of a seed molecule from the PubChem dataset. The corresponding voxelized representations of these molecules are shown in Panel 1265. As shown in FIG. 12B, whether operating in the latent voxelized space or the discrete voxelized space, the molecule design computation model 115 is also able to generate stable, valid, and unique molecules that also closely resemble seed molecules from the PubChem dataset.

Table 7 below further depicts the seeded generation results (averaged over 5 repeats) on the PubChem dataset.

TABLE 7

| | tan | stable | stable | stable | | | tan | stable | stable | stable | |
|---|---|---|---|---|---|---|---|---|---|---|---|
| steps | sim. | mol | sanit. | atom | valid | steps | sim. | mol | sanit. | atom | valid |
| discrete | %_{↑} | %_{↑} | %_{↑} | %_{↑} | %_{↑} | latent | %_{↑} | %_{↑} | %_{↑} | %_{↑} | %_{↑} |
| 5 | 10.95 | 43.73 | 92.45 | 95.6 | 97.11 | 10 | 35.1 | 4.18 | 95.18 | 75.64 | 98.86 |
| 10 | 9.74 | 56.53 | 92.08 | 96.89 | 96.49 | 20 | 32.19 | 4.68 | 95.44 | 76.43 | 98.80 |
| 50 | 9.75 | 74.00 | 90.73 | 98.44 | 95.17 | 50 | 25.62 | 5.51 | 95.64 | 78.35 | 98.36 |
| 100 | 9.81 | 76.18 | 89.93 | 98.62 | 94.81 | 100 | 18.38 | 5.49 | 96.16 | 80.04 | 97.88 |
| 200 | 9.86 | 77.8 | 90.44 | 98.77 | 94.68 | 200 | 12.58 | 5.95 | 95.72 | 79.77 | 96.72 |

FIG. 12C depicts the molecular graphs of additional examples of molecules generated at steps (or sampling iterations) 10, 20, 50, 100, and 200 by the molecule design computation model 115 operating in the latent voxelized space and updating an embedding of the voxelized representation of two real drug seed molecules. The molecule graphs of some example molecules generated at a random selection of steps (or sampling iterations) by the molecule design computation model operating in the latent voxelized space and updating the embedding of a random molecule (e.g., a molecule with a random selection of atomic types and/or positions) are shown in FIG. 12D.

Table 8 below depicts the seeded generation results (averaged over 5 repeats) on five real drugs.

TABLE 8

| steps | | | | | |
|---|---|---|---|---|---|
| (sampling | tan. | stable | stable | stable | |
| iteration) | sim. %_{↑} | mol. %_{↑} | sanit. %_{↑} | atom %_{↑} | valid %_{↑} |
| 5 | 42.4 | 2.22 | 75.56 | 78.85 | 75.56 |
| 10 | 26.93 | 0 | 77.78 | 79.36 | 77.78 |
| 20 | 28.85 | 0 | 77.78 | 80.73 | 77.78 |
| 50 | 23.00 | 0 | 80.00 | 82.33 | 80.00 |
| 100 | 17.99 | 0 | 86.67 | 83.56 | 86.67 |
| 200 | 13.77 | 0 | 91.11 | 83.7 | 91.11 |

FIG. 13 depicts a graph 1300 illustrating a comparison of the number of stable, valid, and unique molecules generated by the molecule design computation model 115 operating in the latent voxelized space, the molecule design computation model 115 operating in the discrete voxelized space, and by a state-of-the-art generative model over time. As shown in FIG. 13, whether operating in the latent voxelized space or the discrete voxelized space, the molecule design computation model 115 is able to generate a much larger number of stable, valid, and unique molecules than the state-of-the-art generative model. Furthermore, the molecule design computation model 115 may generate a larger number of stable, valid, and unique molecules when operating in the latent voxelized space than when operating in the discrete voxelized space.

Table 9 below shows a comparison of the generative performance (averaged over generation of 10,000 molecules repeated 3 times) on Geometric Ensemble of Molecules (GEOM) Drugs of the molecule design computation model 115 performing de novo generation in latent voxelized space (MDCMₗₐₜₑₙₜ), the molecule design computation model 115 performing de novo generation in discrete voxelized space (MDCM_{discrete}), and a state-of-the art generative model EDM.

TABLE 9

| | stable | stable | stable | | | | | | bond | bond | |
|---|---|---|---|---|---|---|---|---|---|---|---|
| | mol | sanit. | atom | valid | unique | valency | atom | bond | len | ang | avg. t |
| | %↑ | %↑ | %↑ | %↑ | %↑ | W₁↓ | TV↓ | TV↓ | W₁↓ | W₁↓ | [s/mol] |
| data | 99.9 | — | 99.9 | 99.8 | 100.0 | 0.001 | 0.001 | 0.025 | 0.00 | 0.05 | — |
| EDM | 40.3 | — | 97.8 | 87.8 | 99.9 | 0.285 | 0.212 | 0.048 | 0.002 | 6.42 | 9.35 |
| MDCM_{D} | 75.0 | — | 98.1 | 93.4 | 99.1 | 0.254 | 0.033 | 0.036 | 0.002 | 0.64 | 7.55 |
| | (±1.0) | | (±.3) | (±0.5) | (±0.2) | (±.003) | (±.041) | (±.006) | (±.001) | (±.13) | |
| MDCM_{L} | 1.19 | 94.65 | 78.05 | 94.81 | 99.91 | 0.29 | .40 | .11 | .01 | 13.29 | 0.71 |
| | (±.62) | (±.54) | (±1.24) | (±.58) | (±.08) | (±.02) | (±.01) | (±.02) | (±.00) | (±.30) | |

Table 10 below shows a comparison of the generative performance (averaged over generation of 10,000 molecules repeated 3 times) on QM9 Drugs of the molecule design computation model 115 performing de novo generation in latent voxelized space (MDCMₗₐₜₑₙₜ), the molecule design computation model 115 performing de novo generation in discrete voxelized space (MDCM_{discrete}), and the state-of-the art generative models GSchNet and EDM.

TABLE 10

| | stable | stable | stable | | | | | | bond | bond |
|---|---|---|---|---|---|---|---|---|---|---|
| | mol | sanit. | atom | valid | unique | valency | atom | bond | len | ang |
| | %_{↑} | % | %_{↑} | %_{↑} | %_{↑} | W_{1↓} | TV_{↓} | TV_{↓} | W_{1↓} | W_{1↓} |
| data | 98.7 | — | 99.8 | 98.9 | 99.9 | 0.001 | 0.003 | 0.000 | 0.000 | 0.120 |
| GschNet | 92.0 | — | 98.7 | 98.1 | 94.5 | 0.049 | 0.042 | 0.041 | 0.005 | 1.68 |
| EDM | 97.9 | | 99.8 | 99.0 | 98.5 | 0.011 | 0.021 | 0.002 | 0.001 | 0.44 |
| MDCM_{D} | 89.3 | | 99.2 | 98.7 | 92.1 | 0.023 | 0.029 | 0.009 | 0.003 | 1.96 |
| | (±0.6) | | (±0.1) | (±0.1) | (±.3) | (±.3) | (±.009) | (±.002) | (±.002) | (±.04) |
| MDCM_{L} | 67.01 | 91.04 | 97.55 | 91.05 | 89.93 | 0.24 | .25 | .18 | .01 | 11.63 |
| | (±.38) | (±.17) | (±0.04) | (±.17) | (±.32) | (±0) | (±0) | (±0) | (±0) | (±0.03) |

FIG. 14 depicts a block diagram illustrating an example of a computing system 1400, in accordance with some example embodiments. Referring to FIGS. 1-14, the computing system 1400 may be used to implement the molecule design engine 110, the training engine 120, the client device 130, and/or any components therein.

As shown in FIG. 14, the computing system 1400 can include a processor 1410, a memory 1420, a storage device 1430, and input/output devices 1440. The processor 1410, the memory 1420, the storage device 1430, and the input/output devices 1440 can be interconnected via a system bus 1450. The processor 1410 is capable of processing instructions for execution within the computing system 1400. Such executed instructions can implement one or more components of, for example, the molecule design engine 110, the analysis engine 120, the client device 130, and/or the like. In some example embodiments, the processor 1410 can be a single-threaded processor. Alternately, the processor 1410 can be a multi-threaded processor. The processor 1410 is capable of processing instructions stored in the memory 1420 and/or on the storage device 1430 to display graphical information for a user interface provided via the input/output device 1440.

The memory 1420 is a computer readable medium such as volatile or non-volatile that stores information within the computing system 1400. The memory 1420 can store data structures representing configuration object databases, for example. The storage device 1430 is capable of providing persistent storage for the computing system 1400. The storage device 1430 can be a floppy disk device, a hard disk device, an optical disk device, or a tape device, or other suitable persistent storage means. The input/output device 1440 provides input/output operations for the computing system 1400. In some example embodiments, the input/output device 1440 includes a keyboard and/or pointing device. In various implementations, the input/output device 1440 includes a display unit for displaying graphical user interfaces.

According to some example embodiments, the input/output device 1440 can provide input/output operations for a network device. For example, the input/output device 1440 can include Ethernet ports or other networking ports to communicate with one or more wired and/or wireless networks (e.g., a local area network (LAN), a wide area network (WAN), the Internet).

In some example embodiments, the computing system 1400 can be used to execute various interactive computer software applications that can be used for organization, analysis and/or storage of data in various formats. Alternatively, the computing system 1400 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1440. The user interface can be generated and presented to a user by the computing system 1400 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desired results. Other implementations may be within the scope of the following claims.

What is claimed is:

1. A system, comprising:
at least one data processor; and
at least one memory storing instructions, which when executed by the at least one data processor, result in operations comprising:
encoding a voxelized representation of an input molecule to generate an embedding of the input molecule having a fewer quantity of features than the voxelized representation of the input molecule;
applying a molecule design computation model to generate an updated embedding by at least updating the embedding of the input molecule,
where the molecule design computation model has been trained to approximate a data distribution of molecules exhibiting one or more desirable properties,
wherein the molecule design computation model updates the embedding of the input molecule to increase a likelihood of the updated embedding being within the data distribution,
where the molecule design computation model is trained by at least applying the molecule design computation model to operate on a corrupted embedding of a voxelized representation of a sample molecule exhibiting the one or more desired properties, and
where the training includes applying the molecule design computation model to recover, from the corrupted embedding, an embedding of the voxelized representation of the sample molecule; and
generating a voxelized representation of an output molecule by at least decoding the updated embedding.

2. The system of claim 1, wherein the data distribution is a noisy data distribution populated by noisy embeddings of a plurality of voxelized representations of the molecules exhibiting the one or more desirable properties, and wherein the voxelized representation is further generated by denoising a noisy voxelized representation of the output molecule generated by the decoding of the updated embedding.

3. (canceled)

4. The system of claim 1, wherein the embedding of the input molecule comprises a discrete latent embedding vector generated by quantizing a corresponding continuous latent embedding, and wherein the quantizing includes matching the corresponding continuous latent embedding to a vector in a codebook of embeddings by a nearest neighbor lookup.

5. The system of claim 1, wherein the voxelized representation of the input molecule is encoded by at least compressing a plurality of atomic density values comprising the voxelized representation of the input molecule such that the embedding of the input molecule includes fewer features than the voxelized representation of the input molecule.

6. The system of claim 1, wherein the voxelized representation of the input molecule includes a plurality of voxels organized into a three-dimensional voxel grid, wherein each atom in the input molecule is represented as a continuous density across one or more voxels in the three-dimensional voxel grid, and wherein each voxel in the three-dimensional voxel grid is associated with a value indicative of an atomic density at a corresponding location.

7. The system of claim 6, wherein the continuous density of each atom in the input molecule is centered at a center of each atom, and wherein a first voxel located distanced from any atoms in the input molecule is associated with a lower atomic density value than a second voxel located proximate to the center of an atom in the input molecule.

8. (canceled)

9. The system of claim 1, wherein the voxelized representation of the input molecule includes one or more channels, and wherein each channel corresponds to a type of atom present in the input molecule.

10. The system of claim 1, wherein the voxelized representation of the input molecule jointly represents a type and a position of one or more atoms present in the input molecule.

11. The system of claim 1, wherein the embedding of the input molecule is updated based at least on a function parameterized by a plurality parameters of the molecule design computation model, wherein the function comprises a score function that outputs a value indicative of a local change in a density of the data distribution at a location of the updated embedding.

12. (canceled)

13. The system of claim 1, wherein the molecule design computation model updates the embedding of the input molecule by at least
applying the molecule design computation model to update the embedding of the input molecule thereby generating a first updated embedding,
applying the molecule design computation model to update the embedding of the input molecule thereby generating a second updated embedding,
applying a function parameterized by a plurality of parameters of the molecule design computation model to determine (i) a first value indicative of a first local change in a density of the data distribution at a first location occupied by the first updated embedding and (ii) a second value indicative of a second local change in the density of the data distribution at a second location occupied by the second updated embedding, and
applying the molecule design computation model to further update, based at least on the first value and the second value, the first updated embedding instead of the second updated embedding.

14. The system of claim 13, wherein the molecule design computation model is applied to further update the first updated embedding until one or more criteria are met, and wherein the one or more criteria include at least one of (i) performing a threshold quantity of iterations of updates to the embedding of the input molecule, (ii) the first value of the first updated embedding satisfying one or more thresholds, and (iii) generating a threshold quantity of output molecules.

15. The system of claim 13, wherein the molecule design computation model is applied to further modify the first updated embedding instead of the second updated embedding based at least on the first value and the second value indicating that the first updated embedding has a higher likelihood within the data distribution than the second updated embedding.

16. The system of claim 13, wherein the molecule design computation model is applied to further modify the first updated embedding instead of the second updated embedding based at least on the first value and the second value indicating that the first updated embedding is sampled from a higher density region of the data distribution than the second updated embedding.

17. The system of claim 1, wherein the operations further comprise:
translating the voxelized representation of the output molecule into a one-dimensional representation of the output molecule and/or a two-dimensional representation of the output molecule.

18. The system of claim 17, wherein the voxelized representation of the output molecule is translated by at least
determining a position of one or more atoms in the output molecule by at least detecting one or more peaks in a plurality of atomic density values comprising the voxelized representation of the output molecule, and
determining, based at least the positions of the one or more atoms, one or more interconnecting bonds.

19. A computer-implemented method, comprising:
encoding a voxelized representation of an input molecule to generate an embedding of the input molecule having a fewer quantity of features than the voxelized representation of the input molecule;
applying a molecule design computation model to generate an updated embedding by at least updating the embedding of the input molecule,
where the molecule design computation model has been trained to approximate a data distribution of molecules exhibiting one or more desirable properties,
wherein the molecule design computation model updates the embedding of the input molecule to increase a likelihood of the updated embedding being within the data distribution,
where the molecule design computation model is trained by at least applying the molecule design computation model to operate on a corrupted embedding of a voxelized representation of a sample molecule exhibiting the one or more desired properties, and
where the training includes applying the molecule design computation model to recover, from the corrupted embedding, an embedding of the voxelized representation of the sample molecule; and
generating a voxelized representation of an output molecule by at least decoding the updated embedding.

20. A non-transitory computer readable medium storing instructions, which when executed by at least one data processor, result in operations comprising:
encoding a voxelized representation of an input molecule to generate an embedding of the input molecule having a fewer quantity of features than the voxelized representation of the input molecule;
applying a molecule design computation model to generate an updated embedding by at least updating the embedding of the input molecule,
where the molecule design computation model has been trained to approximate a data distribution of molecules exhibiting one or more desirable properties,
wherein the molecule design computation model updates the embedding of the input molecule to increase a likelihood of the updated embedding being within the data distribution,
where the molecule design computation model is trained by at least applying the molecule design computation model to operate on a corrupted embedding of a voxelized representation of a sample molecule exhibiting the one or more desired properties, and
where the training includes applying the molecule design computation model to recover, from the corrupted embedding, an embedding of the voxelized representation of the sample molecule; and
generating a voxelized representation of an output molecule by at least decoding the updated embedding.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. The system of claim 1, wherein the training of the molecule design computation model includes
applying the molecule design computation model having a first adjustment to generate a first recovered embedding of the noisy voxelized representation of the sample molecule,
determining a first mean squared error (MSE) quantifying a first difference between the first recovered embedding and the uncorrupted embedding of noisy voxelized representation of the sample molecule,
applying the molecule design computation model having a second adjustment to generate a second recovered embedding of the noisy voxelized representation of the sample molecule,
determining a second mean squared error (MSE) quantifying a second difference between the second recovered embedding and the uncorrupted embedding of noisy voxelized representation of the sample molecule, and
upon determining that the first mean squared error (MSE) is less than the second mean squared error (MSE), further adjusting the molecule design computation model having the first adjustment instead of the second adjustment.

36. The system of claim 35, wherein the molecule design computation model is further adjusted until one or more criteria are met, and wherein the one or more criteria include at least one of (i) performing a threshold quantity of iterations of adjustments to the molecule design computation model, and (ii) generating a recovered embedding exhibiting a threshold mean squared error (MSE) value.

37. The system of claim 1, wherein the operations further comprise:
training an autoencoder comprising an encoder and a decoder, wherein the training of the autoencoder includes training the encoder to generate an embedding of a noisy voxelized representation of the sample molecule such that the decoder is able to recover the voxelized representation of the sample molecule from the embedding of the noisy voxelized representation of the sample molecule.

38. (canceled)

39. (canceled)

40. (canceled)
